SEQUENCE LISTING (400 sequences)

SEQ ID NO: 1 - protein, 5 aa, Artificial Sequence
  Synthetic Construct
  MISC_FEATURE (2)..(4): Xaa is any amono acid other than Cys
  MISC_FEATURE (5)..(5): Xaa is Ser or Thr
GXXXX

SEQ ID NO: 2 - protein, 10 aa, Artificial Sequence
  Synthetic Construct
  MISC_FEATURE (2)..(4): Xaa is any amino acid other than Cys
  MISC_FEATURE (5)..(5): Xaa is Ser or Thr
  MISC_FEATURE (6)..(10): Xaa is any basic amino acid
GXXXXXXXXX

SEQ ID NO: 3 - protein, 5 aa, Artificial Sequence
  Synthetic Construct
  MISC_FEATURE (3)..(5): Xaa is any amino acid
  MISC_FEATURE (6)..(6): Xaa is any amino acid
  MISC_FEATURE (6)..(6): Xaa is any Ser or Thr
MGXXX

SEQ ID NO: 4 - protein, 1367 aa, Artificial Sequence
  Synthetic Construct
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial Sequence
  Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1368 aa, Artificial Sequence
  Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - DNA, 58 bp, Artificial Sequence
  Synthetic Construct
ccggactggacacatacccatataactcgagttatatgggtatgtgtccagttttttg

SEQ ID NO: 8 - DNA, 57 bp, Artificial Sequence
  Synthetic Construct
ccgggccttatagaggtaatacatactcgagtatgtattacctctataaggcttttg

SEQ ID NO: 9 - protein, 30 aa, Artificial Sequence
  Synthetic Construct
MGNIFANLFKGLFGKKEMRILMVGLDAAGK

SEQ ID NO: 10 - protein, 30 aa, Artificial Sequence
  Synthetic Construct
MGLTISSLFSRLFGKKQMRILMVGLDAAGK

SEQ ID NO: 11 - protein, 30 aa, Artificial Sequence
  Synthetic Construct
MGKVLSKIFGNKEMWILMLGLDAAGKTTIL

SEQ ID NO: 12 - protein, 30 aa, Artificial Sequence
  Synthetic Construct
MGCTVSAEDKAAAERSKMIDKNLREDGEKA

SEQ ID NO: 13 - protein, 30 aa, Artificial Sequence
  Synthetic Construct
MGCTLSAEDKAAVERSKMIDRNLREDGEKA

SEQ ID NO: 14 - protein, 30 aa, Artificial Sequence
  Synthetic Construct
MGISRDNWHKRRKTGGKRKPYHKKRKYELG

SEQ ID NO: 15 - protein, 30 aa, Artificial Sequence
  Synthetic Construct
MGDVLSTHLDDARRQHIAEKTGKILTEFLQ

SEQ ID NO: 16 - protein, 30 aa, Artificial Sequence
  Synthetic Construct
MGCCYSSENEDSDQDREERKLLLDPSSPPT

SEQ ID NO: 17 - protein, 30 aa, Artificial Sequence
  Synthetic Construct
MGNCHTVGPNEALVVSGGCCGSDYKQYVFG

SEQ ID NO: 18 - protein, 30 aa, Artificial Sequence
  Synthetic Construct
MGLTVSALFSRIFGKKQMRILMVGLDAAGK

SEQ ID NO: 19 - protein, 30 aa, Artificial Sequence
  Synthetic Construct
MGAYKYIQELWRKKQSDVMRFLLRVRCWQY

SEQ ID NO: 20 - protein, 30 aa, Artificial Sequence
  Synthetic Construct
MGCIKSKENKSPAIKYRPENTPEPVSTSVS

SEQ ID NO: 21 - protein, 30 aa, Artificial Sequence
  Synthetic Construct
MGNLLKVLTCTDLEQGPNFFLDFENAQPTE

SEQ ID NO: 22 - protein, 30 aa, Artificial Sequence
  Synthetic Construct
MGKSASKQFHNEVLKAHNEYRQKHGVPPLK

SEQ ID NO: 23 - protein, 30 aa, Artificial Sequence; identical to SEQ ID NO: 13
  Synthetic Construct

SEQ ID NO: 24 - protein, 30 aa, Artificial Sequence
  Synthetic Construct
MGLLSILRKLKSAPDQEVRILLLGLDNAGK

SEQ ID NO: 25 - protein, 30 aa, Artificial Sequence
  Synthetic Construct
MGLLTILKKMKQKERELRLLMLGLDNAGKT

SEQ ID NO: 26 - protein, 30 aa, Artificial Sequence
  Synthetic Construct
MGNLFGRKKQSRVTEQDKAILQLKQQRDKL

SEQ ID NO: 27 - protein, 30 aa, Artificial Sequence
  Synthetic Construct
MGSRASTLLRDEELEEIKKETGFSHSQITR

SEQ ID NO: 28 - protein, 30 aa, Artificial Sequence
  Synthetic Construct
MGCCSSASSAAQSSKREWKPLEDRSCTDIP

SEQ ID NO: 29 - protein, 30 aa, Artificial Sequence
  Synthetic Construct
MGCIKSKGKDSLSDDGVDLKTQPVRNTERT

SEQ ID NO: 30 - protein, 30 aa, Artificial Sequence
  Synthetic Construct
MGSQSSKAPRGDVTAEEAAGASPAKANGQE

SEQ ID NO: 31 - protein, 30 aa, Artificial Sequence
  Synthetic Construct
MGCFFSKRRKADKESRPENEEERPKQYSWD

SEQ ID NO: 32 - protein, 30 aa, Artificial Sequence
  Synthetic Construct
MGAQFSKTAAKGEAAAERPGEAAVASSPSK

SEQ ID NO: 33 - protein, 30 aa, Artificial Sequence
  Synthetic Construct
MGNSALRAHVETAQKTGVFQLKDRGLTEFP

SEQ ID NO: 34 - protein, 30 aa, Artificial Sequence
  Synthetic Construct
MGKQNSKLRPEVLQDLRENTEFTDHELQEW

SEQ ID NO: 35 - protein, 30 aa, Artificial Sequence
  Synthetic Construct
MGAQLSTLGHMVLFPVWFLYSLLMKLFQRS

SEQ ID NO: 36 - protein, 30 aa, Artificial Sequence
  Synthetic Construct
MGSVLGLCSMASWIPCLCGSAPCLLCRCCP

SEQ ID NO: 37 - protein, 30 aa, Artificial Sequence
  Synthetic Construct
MGSNKSKPKDASQRRRSLEPAENVHGAGGG

SEQ ID NO: 38 - protein, 30 aa, Artificial Sequence
  Synthetic Construct
MGGFFSSIFSSLFGTREMRILILGLDGAGK

SEQ ID NO: 39 - protein, 30 aa, Artificial Sequence
  Synthetic Construct
MGGKLSKKKKGYNVNDEKAKEKDKKAEGAA

SEQ ID NO: 40 - protein, 30 aa, Artificial Sequence
  Synthetic Construct
MGGTTSTRRVTFEADENENITVVKGIRLSE

SEQ ID NO: 41 - protein, 30 aa, Artificial Sequence
  Synthetic Construct
MGNAGSMDSQQTDFRAHNVPLKLPMPEPGE

SEQ ID NO: 42 - protein, 30 aa, Artificial Sequence
  Synthetic Construct
MGKSNSKLKPEVVEELTRKTYFTEKEVQQW

SEQ ID NO: 43 - protein, 30 aa, Artificial Sequence
  Synthetic Construct
MGGSASSQLDEGKCAYIRGKTEAAIKNFSP

SEQ ID NO: 44 - protein, 30 aa, Artificial Sequence
  Synthetic Construct
MGLCFPCPGESAPPTPDLEEKRAKLAEAAE

SEQ ID NO: 45 - protein, 30 aa, Artificial Sequence
  Synthetic Construct
MGLFGKTQEKPPKELVNEWSLKIRKEMRVV

SEQ ID NO: 46 - protein, 30 aa, Artificial Sequence
  Synthetic Construct
MGGSGSRLSKELLAEYQDLTFLTKQEILLA

SEQ ID NO: 47 - protein, 30 aa, Artificial Sequence
  Synthetic Construct
MGNAAAAKKGSEQESVKEFLAKAKEDFLKK

SEQ ID NO: 48 - protein, 30 aa, Artificial Sequence
  Synthetic Construct
MGNTTSCCVSSSPKLRRNAHSRLESYRPDT

SEQ ID NO: 49 - protein, 30 aa, Artificial Sequence
  Synthetic Construct
MGSSQSVEIPGGGTEGYHVLRVQENSPGHR

SEQ ID NO: 50 - protein, 30 aa, Artificial Sequence
  Synthetic Construct
MGNQLAGIAPSQILSVESYFSDIHDFEYDK

SEQ ID NO: 51 - protein, 30 aa, Artificial Sequence
  Synthetic Construct
MGCGLNKLEKRDEKRPGNIYSTLKRPQVET

SEQ ID NO: 52 - protein, 30 aa, Artificial Sequence
  Synthetic Construct
MGRESRHYRKRSASRGRSGSRSRSRSPSDK

SEQ ID NO: 53 - protein, 30 aa, Artificial Sequence
  Synthetic Construct
MGNAQERPSETIDRERKRLVETLQADSGLL

SEQ ID NO: 54 - protein, 30 aa, Artificial Sequence
  Synthetic Construct
MGKSESQMDITDINTPKPKKKQRWTPLEIS

SEQ ID NO: 55 - protein, 30 aa, Artificial Sequence
  Synthetic Construct
MGNAATAKKGSEVESVKEFLAKAKEDFLKK

SEQ ID NO: 56 - protein, 30 aa, Artificial Sequence
  Synthetic Construct
MGSTDSKLNFRKAVIQLTTKTQPVEATDDA

SEQ ID NO: 57 - protein, 30 aa, Artificial Sequence
  Synthetic Construct
MGNLESAEGVPGEPPSVPLLLPPGKMPMPE

SEQ ID NO: 58 - protein, 30 aa, Artificial Sequence
  Synthetic Construct
MGAYLSQPNTVKCSGDGVGAPRLPLPYGFS

SEQ ID NO: 59 - protein, 30 aa, Artificial Sequence
  Synthetic Construct
MGKSLSHLPLHSSKEDAYDGVTSENMRNGL

SEQ ID NO: 60 - protein, 30 aa, Artificial Sequence
  Synthetic Construct
MGCTLSAEERAALERSKAIEKNLKEDGISA

SEQ ID NO: 61 - protein, 30 aa, Artificial Sequence
  Synthetic Construct
MGASGSKARGLWPFASAAGGGGSEAAGAEQ

SEQ ID NO: 62 - protein, 30 aa, Artificial Sequence
  Synthetic Construct
MGETMSKRLKLHLGGEAEMEERAFVNPFPD

SEQ ID NO: 63 - protein, 30 aa, Artificial Sequence
  Synthetic Construct
MGAGSSTEQRSPEQPPEGSSTPAEPEPSGG

SEQ ID NO: 64 - protein, 30 aa, Artificial Sequence
  Synthetic Construct
MGCGCSSHPEDDWMENIDVCENCHYPIVPL

SEQ ID NO: 65 - protein, 30 aa, Artificial Sequence
  Synthetic Construct
MGNRHAKASSPQGFDVDRDAKKLNKACKGM

SEQ ID NO: 66 - protein, 30 aa, Artificial Sequence
  Synthetic Construct
MGCVQCKDKEATKLTEERDGSLNQSSGYRY

SEQ ID NO: 67 - protein, 30 aa, Artificial Sequence
  Synthetic Construct
MGNGMCSRKQKRIFQTLLLLTVVFGFLYGA

SEQ ID NO: 68 - protein, 30 aa, Artificial Sequence
  Synthetic Construct
MGNEASYPLEMCSHFDADEIKRLGKRFKKL

SEQ ID NO: 69 - protein, 30 aa, Artificial Sequence
  Synthetic Construct
MGKQNSKLAPEVMEDLVKSTEFNEHELKQW

SEQ ID NO: 70 - protein, 30 aa, Artificial Sequence
  Synthetic Construct
MGQCVTKCKNPSSTLGSKNGDREPSNKSHS

SEQ ID NO: 71 - protein, 30 aa, Artificial Sequence
  Synthetic Construct
MGNGESQLSSVPAQKLGWFIQEYLKPYEEC

SEQ ID NO: 72 - protein, 30 aa, Artificial Sequence
  Synthetic Construct
MGAFLDKPKTEKHNAHGAGNGLRYGLSSMQ

SEQ ID NO: 73 - protein, 30 aa, Artificial Sequence
  Synthetic Construct
MGNISSNISAFQSLHIVMLGLDSAGKTTVL

SEQ ID NO: 74 - protein, 30 aa, Artificial Sequence
  Synthetic Construct
MGRKSSKAKEKKQKRLEERAAMDAVCAKVD

SEQ ID NO: 75 - protein, 30 aa, Artificial Sequence
  Synthetic Construct
MGTTASTAQQTVSAGTPFEGLQGSGTMDSR

SEQ ID NO: 76 - protein, 30 aa, Artificial Sequence
  Synthetic Construct
MGNAPSHSSEDEAAAAGGEGWGPHQDWAAV

SEQ ID NO: 77 - protein, 30 aa, Artificial Sequence
  Synthetic Construct
MGSQVSVESGALHVVIVGGGFGGIAAASQL

SEQ ID NO: 78 - protein, 30 aa, Artificial Sequence
  Synthetic Construct
MGQTKSKIKSKYASYLSFIKILLKRGGVKV

SEQ ID NO: 79 - protein, 30 aa, Artificial Sequence
  Synthetic Construct
MGGLFSRWRTKPSTVEVLESIDKEIQALEE

SEQ ID NO: 80 - protein, 30 aa, Artificial Sequence
  Synthetic Construct
MGAAHSASEEVRELEGKTGFSSDQIEQLHR

SEQ ID NO: 81 - protein, 30 aa, Artificial Sequence
  Synthetic Construct
MGSVSSLISGHSFHSKHCRASQYKLRKSSH

SEQ ID NO: 82 - protein, 30 aa, Artificial Sequence
  Synthetic Construct
MGKLHSKPAAVCKRRESPEGDSFAVSAAWA

SEQ ID NO: 83 - protein, 30 aa, Artificial Sequence
  Synthetic Construct
MGNCLKSPTSDDISLLHESQSDRASFGEGT

SEQ ID NO: 84 - protein, 30 aa, Artificial Sequence
  Synthetic Construct
MGAKQSGPAAANGRTRAYSGSDLPSSSSGG

SEQ ID NO: 85 - protein, 30 aa, Artificial Sequence
  Synthetic Construct
MGSRVSREDFEWVYTDQPHADRRREILAKY

SEQ ID NO: 86 - protein, 30 aa, Artificial Sequence
  Synthetic Construct
MGSCCSCPDKDTVPDNHRNKFKVINVDDDG

SEQ ID NO: 87 - protein, 30 aa, Artificial Sequence
  Synthetic Construct
MGGRSSCEDPGCPRDEERAPRMGCMKSKFL

SEQ ID NO: 88 - protein, 30 aa, Artificial Sequence
  Synthetic Construct
MGALVIRGIRNFNLENRAEREISKMKPSVA

SEQ ID NO: 89 - protein, 30 aa, Artificial Sequence
  Synthetic Construct
MGAHLVRRYLGDASVEPDPLQMPTFPPDYG

SEQ ID NO: 90 - protein, 30 aa, Artificial Sequence
  Synthetic Construct
MGNGLSDQTSILSNLPSFQSFHIVILGLDC

SEQ ID NO: 91 - protein, 30 aa, Artificial Sequence
  Synthetic Construct
MGLLDRLSVLLGLKKKEVHVLCLGLDNSGK

SEQ ID NO: 92 - protein, 30 aa, Artificial Sequence
  Synthetic Construct
MGCMKSKQTFPFPTIYEGEKQHESEEPFMP

SEQ ID NO: 93 - protein, 30 aa, Artificial Sequence
  Synthetic Construct
MGSTESSEGRRVSFGVDEEERVRVLQGVRL

SEQ ID NO: 94 - protein, 30 aa, Artificial Sequence
  Synthetic Construct
MGSTLGCHRSIPRDPSDLSHSRKFSAACNF

SEQ ID NO: 95 - protein, 30 aa, Artificial Sequence; identical to SEQ ID NO: 78
  Synthetic Construct

SEQ ID NO: 96 - protein, 30 aa, Artificial Sequence
  Synthetic Construct
MGCRQSSEEKEAARRSRRIDRHLRSESQRQ

SEQ ID NO: 97 - protein, 30 aa, Artificial Sequence
  Synthetic Construct
MGARGALLLALLLARAGLRKPESQEAAPLS

SEQ ID NO: 98 - protein, 30 aa, Artificial Sequence
  Synthetic Construct
MGSGASAEDKELAKRSKELEKKLQEDADKE

SEQ ID NO: 99 - protein, 30 aa, Artificial Sequence
  Synthetic Construct
MGSGISSESKESAKRSKELEKKLQEDAERD

SEQ ID NO: 100 - protein, 30 aa, Artificial Sequence
  Synthetic Construct
MGSILSRRIAGVEDIDIQANSAYRYPPKSG

SEQ ID NO: 101 - protein, 30 aa, Artificial Sequence
  Synthetic Construct
MGQKASQQLALKDSKEVPVVCEVVSEAIVH

SEQ ID NO: 102 - protein, 30 aa, Artificial Sequence
  Synthetic Construct
MGCGLRKLEDPDDSSPGKIFSTLKRPQVET

SEQ ID NO: 103 - protein, 30 aa, Artificial Sequence
  Synthetic Construct
MGSENSALKSYTLREPPFTLPSGLAVYPAV

SEQ ID NO: 104 - protein, 30 aa, Artificial Sequence
  Synthetic Construct
MGSLPSRRKSLPSPSLSSSVQGQGPVTMEA

SEQ ID NO: 105 - protein, 30 aa, Artificial Sequence
  Synthetic Construct
MGHALCVCSRGTVIIDNKRYLFIQKLGEGG

SEQ ID NO: 106 - protein, 30 aa, Artificial Sequence
  Synthetic Construct
MGVNQSVGFPPVTGPHLVGCGDVMEGQNLQ

SEQ ID NO: 107 - protein, 30 aa, Artificial Sequence
  Synthetic Construct
MGQQVGRVGEAPGLQQPQPRGIRGSSAARP

SEQ ID NO: 108 - protein, 30 aa, Artificial Sequence
  Synthetic Construct
MGQLCCFPFSRDEGKISELESSSSAVLQRY

SEQ ID NO: 109 - protein, 30 aa, Artificial Sequence
  Synthetic Construct
MGNTTTKFRKALINGDENLACQIYENNPQL

SEQ ID NO: 110 - protein, 30 aa, Artificial Sequence
  Synthetic Construct
MGNIFGNLLKSLIGKKEMRILMVGLDAAGK

SEQ ID NO: 111 - protein, 30 aa, Artificial Sequence
  Synthetic Construct
MGSVNSRGHKAEAQVVMMGLDSAGKTTLLY

SEQ ID NO: 112 - protein, 30 aa, Artificial Sequence
  Synthetic Construct
MGSLGSKNPQTKQAQVLLLGLDSAGKSTLL

SEQ ID NO: 113 - protein, 29 aa, Artificial Sequence
  Synthetic Construct
MGNIFEKLFKSLLGKKKMRILILSLDTAG

SEQ ID NO: 114 - protein, 30 aa, Artificial Sequence
  Synthetic Construct
MGNHLTEMAPTASSFLPHFQALHVVVIGLD

SEQ ID NO: 115 - protein, 30 aa, Artificial Sequence
  Synthetic Construct
MGILFTRIWRLFNHQEHKVIIVGLDNAGKT

SEQ ID NO: 116 - protein, 30 aa, Artificial Sequence
  Synthetic Construct
MGLIFAKLWSLFCNQEHKVIIVGLDNAGKT

SEQ ID NO: 117 - protein, 30 aa, Artificial Sequence
  Synthetic Construct
MGQLIAKLMSIFGNQEHTVIIVGLDNEGKT

SEQ ID NO: 118 - protein, 30 aa, Artificial Sequence
  Synthetic Construct
MGCGGSRADAIEPRYYESWTRETESTWLTY

SEQ ID NO: 119 - protein, 30 aa, Artificial Sequence
  Synthetic Construct
MGLVSSKKPDKEKPIKEKDKGQWSPLKVSA

SEQ ID NO: 120 - protein, 30 aa, Artificial Sequence
  Synthetic Construct
MGSEQSSEAESRPNDLNSSVTPSPAKHRAK

SEQ ID NO: 121 - protein, 30 aa, Artificial Sequence
  Synthetic Construct
MGNEVSLEGGAGDGPLPPGGAGPGPGPGPG

SEQ ID NO: 122 - protein, 30 aa, Artificial Sequence
  Synthetic Construct
MGANASNYPHSCSPRVGGNSQAQQTFIGTS

SEQ ID NO: 123 - protein, 30 aa, Artificial Sequence
  Synthetic Construct
MGCTPSHSDLVNSVAKSGIQFLKKPKAIRP

SEQ ID NO: 124 - protein, 30 aa, Artificial Sequence
  Synthetic Construct
MGGGDGAAFKRPGDGARLQRVLGLGSRREP

SEQ ID NO: 125 - protein, 30 aa, Artificial Sequence
  Synthetic Construct
MGNCAKRPWRRGPKDPLQWLGSPPRGSCPS

SEQ ID NO: 126 - protein, 30 aa, Artificial Sequence
  Synthetic Construct
MGCRHSRLSSCKPPKKKRQEPEPEQPPRPE

SEQ ID NO: 127 - protein, 30 aa, Artificial Sequence
  Synthetic Construct
MGTVLSLSPSYRKATLFEDGAATVGHYTAV

SEQ ID NO: 128 - protein, 30 aa, Artificial Sequence
  Synthetic Construct
MGTVLSLSPASSAKGRRPGGLPEEKKKAPP

SEQ ID NO: 129 - protein, 30 aa, Artificial Sequence
  Synthetic Construct
MGSRSSHAAVIPDGDSIRRETGFSQASLLR

SEQ ID NO: 130 - protein, 30 aa, Artificial Sequence
  Synthetic Construct
MGSGSSRSSRTLRRRRSPESLPAGPGAAAL

SEQ ID NO: 131 - protein, 30 aa, Artificial Sequence
  Synthetic Construct
MGNSASRSDFEWVYTDQPHTQRRKEILAKY

SEQ ID NO: 132 - protein, 30 aa, Artificial Sequence
  Synthetic Construct
MGNGMNKILPGLYIGNFKDARDAEQLSKNK

SEQ ID NO: 133 - protein, 30 aa, Artificial Sequence
  Synthetic Construct
MGSNSSRIGDLPKNEYLKKLSGTESISEND

SEQ ID NO: 134 - protein, 30 aa, Artificial Sequence
  Synthetic Construct
MGQALGIKSCDFQAARNNEEHHTKALSSRR

SEQ ID NO: 135 - protein, 30 aa, Artificial Sequence; identical to SEQ ID NO: 78
  Synthetic Construct

SEQ ID NO: 136 - protein, 30 aa, Artificial Sequence; identical to SEQ ID NO: 78
  Synthetic Construct

SEQ ID NO: 137 - protein, 30 aa, Artificial Sequence; identical to SEQ ID NO: 78
  Synthetic Construct

SEQ ID NO: 138 - protein, 30 aa, Artificial Sequence
  Synthetic Construct
MGQTKSKTKSKYASYLSFIKILLKRGGVRV

SEQ ID NO: 139 - protein, 30 aa, Artificial Sequence; identical to SEQ ID NO: 78
  Synthetic Construct

SEQ ID NO: 140 - protein, 30 aa, Artificial Sequence; identical to SEQ ID NO: 78
  Synthetic Construct

SEQ ID NO: 141 - protein, 30 aa, Artificial Sequence; identical to SEQ ID NO: 78
  Synthetic Construct

SEQ ID NO: 142 - protein, 30 aa, Artificial Sequence; identical to SEQ ID NO: 78
  Synthetic Construct

SEQ ID NO: 143 - protein, 30 aa, Artificial Sequence
  Synthetic Construct
MGCVFCKKLEPVATAKEDAGLEGDFRSYGA

SEQ ID NO: 144 - protein, 30 aa, Artificial Sequence
  Synthetic Construct
MGNAAGSAEQPAGPAAPPPKQPAPPKQPMP

SEQ ID NO: 145 - protein, 30 aa, Artificial Sequence
  Synthetic Construct
MGSCCSCLNRDSVPDNHPTKFKVTNVDDEG

SEQ ID NO: 146 - protein, 30 aa, Artificial Sequence; identical to SEQ ID NO: 13
  Synthetic Construct

SEQ ID NO: 147 - protein, 30 aa, Artificial Sequence
  Synthetic Construct
MGLGVSAEQPAGGAEGFHLHGVQENSPAQQ

SEQ ID NO: 148 - protein, 30 aa, Artificial Sequence
  Synthetic Construct
MGNVMEGKSVEELSSTECHQWYKKFMTECP

SEQ ID NO: 149 - protein, 30 aa, Artificial Sequence
  Synthetic Construct
MGQEFSWEEAEAAGEIDVAELQEWYKKFVM

SEQ ID NO: 150 - protein, 30 aa, Artificial Sequence
  Synthetic Construct
MGNGKSIAGDQKAVPTQETHVWYRTFMMEY

SEQ ID NO: 151 - protein, 30 aa, Artificial Sequence
  Synthetic Construct
MGLSPSAPAVAVQASNASASPPSGCPMHEG

SEQ ID NO: 152 - protein, 30 aa, Artificial Sequence; identical to SEQ ID NO: 78
  Synthetic Construct

SEQ ID NO: 153 - protein, 30 aa, Artificial Sequence
  Synthetic Construct
MGNVPSAVKHCLSYQQLLREHLWIGDSVAG

SEQ ID NO: 154 - protein, 30 aa, Artificial Sequence
  Synthetic Construct
MGKQNSKLRPEMLQDLRENTEFSELELQEW

SEQ ID NO: 155 - protein, 30 aa, Artificial Sequence
  Synthetic Construct
MGKTNSKLAPEVLEDLVQNTEFSEQELKQW

SEQ ID NO: 156 - protein, 30 aa, Artificial Sequence
  Synthetic Construct
MGSVRTNRYSIVSSEEDGMKLATMAVANGF

SEQ ID NO: 157 - protein, 30 aa, Artificial Sequence
  Synthetic Construct
MGAAGSSALARFVLLAQSRPGWLGVAALGL

SEQ ID NO: 158 - protein, 30 aa, Artificial Sequence
  Synthetic Construct
MGKQNSKLRPEVMQDLLESTDFTEHEIQEW

SEQ ID NO: 159 - protein, 30 aa, Artificial Sequence
  Synthetic Construct
MGNNFSSIPSLPRGNPSRAPRGHPQNLKDS

SEQ ID NO: 160 - protein, 30 aa, Artificial Sequence
  Synthetic Construct
MGKLQSKHAAAARKRRESPEGDSFVASAYA

SEQ ID NO: 161 - protein, 30 aa, Artificial Sequence
  Synthetic Construct
MGNLKSVAQEPGPPCGLGLGLGLGLCGKQG

SEQ ID NO: 162 - protein, 30 aa, Artificial Sequence
  Synthetic Construct
MGTASSLVSPAGGEVIEDTYGAGGGEACEI

SEQ ID NO: 163 - protein, 20 aa, Artificial Sequence
  Synthetic Construct
LRSEAMSSVAAKVRAARAFG

SEQ ID NO: 164 - protein, 30 aa, Artificial Sequence
  Synthetic Construct
MGGAVSAGEDNDDLIDNLKEAQYIRTERVE

SEQ ID NO: 165 - protein, 30 aa, Artificial Sequence
  Synthetic Construct
MGGAVSAGEDNDELIDNLKEAQYIRTELVE

SEQ ID NO: 166 - protein, 30 aa, Artificial Sequence
  Synthetic Construct
MGQACGHSILCRSQQYPAARPAEPRGQQVF

SEQ ID NO: 167 - protein, 30 aa, Artificial Sequence
  Synthetic Construct
MGVLMSKRQTVEQVQKVSLAVSAFKDGLRD

SEQ ID NO: 168 - protein, 30 aa, Artificial Sequence
  Synthetic Construct
MGNSHCVPQAPRRLRASFSRKPSLKGNRED

SEQ ID NO: 169 - protein, 30 aa, Artificial Sequence
  Synthetic Construct
MGAFLDKPKMEKHNAQGQGNGLRYGLSSMQ

SEQ ID NO: 170 - protein, 30 aa, Artificial Sequence
  Synthetic Construct
MGNEASYPAEMCSHFDNDEIKRLGRRFKKL

SEQ ID NO: 171 - protein, 30 aa, Artificial Sequence
  Synthetic Construct
MGNTSSERAALERHGGHKTPRRDSSGGTKD

SEQ ID NO: 172 - protein, 30 aa, Artificial Sequence
  Synthetic Construct
MGNAPAKKDTEQEESVNEFLAKARGDFLYR

SEQ ID NO: 173 - protein, 30 aa, Artificial Sequence
  Synthetic Construct
MGNGSVKPKHSKHPDGHSGNLTTDALRNKV

SEQ ID NO: 174 - protein, 30 aa, Artificial Sequence
  Synthetic Construct
MGMKHSSRCLLLRRKMAENAAESTEVNSPP

SEQ ID NO: 175 - protein, 30 aa, Artificial Sequence
  Synthetic Construct
MGCGTSKVLPEPPKDVQLDLVKKVEPFSGT

SEQ ID NO: 176 - protein, 30 aa, Artificial Sequence
  Synthetic Construct
MGQDQTKQQIEKGLQLYQSNQTEKALQVWT

SEQ ID NO: 177 - protein, 30 aa, Artificial Sequence
  Synthetic Construct
MGNSKSGALSKEILEELQLNTKFSEEELCS

SEQ ID NO: 178 - protein, 30 aa, Artificial Sequence
  Synthetic Construct
MGSVLSTDSGKSAPASATARALERRRDPEL

SEQ ID NO: 179 - protein, 30 aa, Artificial Sequence
  Synthetic Construct
MGQQISDQTQLVINKLPEKVAKHVTLVRES

SEQ ID NO: 180 - protein, 30 aa, Artificial Sequence
  Synthetic Construct
MGALTSRQHAGVEEVDIPSNSVYRYPPKSG

SEQ ID NO: 181 - protein, 30 aa, Artificial Sequence
  Synthetic Construct
MGNSMKSTPAPAERPLPNPEGLDSDFLAVL

SEQ ID NO: 182 - protein, 30 aa, Artificial Sequence
  Synthetic Construct
MGANTSRKPPVFDENEDVNFDHFEILRAIG

SEQ ID NO: 183 - protein, 30 aa, Artificial Sequence
  Synthetic Construct
MGCGPSQPAEDRRRVRAPKKGWKEEFKADV

SEQ ID NO: 184 - protein, 30 aa, Artificial Sequence
  Synthetic Construct
MGNAESQHVEHEFYGEKHASLGRKHTSRSL

SEQ ID NO: 185 - protein, 30 aa, Artificial Sequence
  Synthetic Construct
MGNSDSQYTLQGSKNHSNTITGAKQIPCSL

SEQ ID NO: 186 - protein, 60 aa, Artificial Sequence
  Synthetic Construct
MGIGKSKINSCPLSLSWGKRHSVDTSPGYHMGIGKSKINSCPLSLSWGKRHSVDTSPGYH

SEQ ID NO: 187 - protein, 30 aa, Artificial Sequence
  Synthetic Construct
MGNSRSRVGRSFCSQFLPEEQAEIDQLFDA

SEQ ID NO: 188 - protein, 30 aa, Artificial Sequence
  Synthetic Construct
MGSQHSAAARPSSCRRKQEDDRDGLLAERE

SEQ ID NO: 189 - protein, 30 aa, Artificial Sequence
  Synthetic Construct
MGSKRGISSRHHSLSSYEIMFAALFAILVV

SEQ ID NO: 190 - protein, 30 aa, Artificial Sequence
  Synthetic Construct
MGGKQSTAARSRGPFPGVSTDDSAVPPPGG

SEQ ID NO: 191 - protein, 30 aa, Artificial Sequence; identical to SEQ ID NO: 10
  Synthetic Construct

SEQ ID NO: 192 - protein, 30 aa, Artificial Sequence; identical to SEQ ID NO: 11
  Synthetic Construct

SEQ ID NO: 193 - protein, 30 aa, Artificial Sequence; identical to SEQ ID NO: 12
  Synthetic Construct

SEQ ID NO: 194 - protein, 30 aa, Artificial Sequence; identical to SEQ ID NO: 13
  Synthetic Construct

SEQ ID NO: 195 - protein, 30 aa, Artificial Sequence; identical to SEQ ID NO: 15
  Synthetic Construct

SEQ ID NO: 196 - protein, 30 aa, Artificial Sequence; identical to SEQ ID NO: 16
  Synthetic Construct

SEQ ID NO: 197 - protein, 30 aa, Artificial Sequence; identical to SEQ ID NO: 17
  Synthetic Construct

SEQ ID NO: 198 - protein, 30 aa, Artificial Sequence; identical to SEQ ID NO: 18
  Synthetic Construct

SEQ ID NO: 199 - protein, 30 aa, Artificial Sequence; identical to SEQ ID NO: 19
  Synthetic Construct

SEQ ID NO: 200 - protein, 30 aa, Artificial Sequence; identical to SEQ ID NO: 20
  Synthetic Construct

SEQ ID NO: 201 - protein, 30 aa, Artificial Sequence; identical to SEQ ID NO: 21
  Synthetic Construct

SEQ ID NO: 202 - protein, 30 aa, Artificial Sequence; identical to SEQ ID NO: 22
  Synthetic Construct

SEQ ID NO: 203 - protein, 30 aa, Artificial Sequence; identical to SEQ ID NO: 13
  Synthetic Construct

SEQ ID NO: 204 - protein, 30 aa, Artificial Sequence; identical to SEQ ID NO: 24
  Synthetic Construct

SEQ ID NO: 205 - protein, 30 aa, Artificial Sequence; identical to SEQ ID NO: 26
  Synthetic Construct

SEQ ID NO: 206 - protein, 30 aa, Artificial Sequence; identical to SEQ ID NO: 27
  Synthetic Construct

SEQ ID NO: 207 - protein, 30 aa, Artificial Sequence; identical to SEQ ID NO: 28
  Synthetic Construct

SEQ ID NO: 208 - protein, 30 aa, Artificial Sequence; identical to SEQ ID NO: 29
  Synthetic Construct

SEQ ID NO: 209 - protein, 30 aa, Artificial Sequence; identical to SEQ ID NO: 30
  Synthetic Construct

SEQ ID NO: 210 - protein, 30 aa, Artificial Sequence; identical to SEQ ID NO: 31
  Synthetic Construct

SEQ ID NO: 211 - protein, 30 aa, Artificial Sequence; identical to SEQ ID NO: 32
  Synthetic Construct

SEQ ID NO: 212 - protein, 30 aa, Artificial Sequence; identical to SEQ ID NO: 33
  Synthetic Construct

SEQ ID NO: 213 - protein, 30 aa, Artificial Sequence; identical to SEQ ID NO: 34
  Synthetic Construct

SEQ ID NO: 214 - protein, 30 aa, Artificial Sequence; identical to SEQ ID NO: 36
  Synthetic Construct

SEQ ID NO: 215 - protein, 30 aa, Artificial Sequence; identical to SEQ ID NO: 38
  Synthetic Construct

SEQ ID NO: 216 - protein, 30 aa, Artificial Sequence; identical to SEQ ID NO: 39
  Synthetic Construct

SEQ ID NO: 217 - protein, 30 aa, Artificial Sequence; identical to SEQ ID NO: 41
  Synthetic Construct

SEQ ID NO: 218 - protein, 30 aa, Artificial Sequence; identical to SEQ ID NO: 43
  Synthetic Construct

SEQ ID NO: 219 - protein, 30 aa, Artificial Sequence; identical to SEQ ID NO: 44
  Synthetic Construct

SEQ ID NO: 220 - protein, 30 aa, Artificial Sequence; identical to SEQ ID NO: 45
  Synthetic Construct

SEQ ID NO: 221 - protein, 30 aa, Artificial Sequence; identical to SEQ ID NO: 46
  Synthetic Construct

SEQ ID NO: 222 - protein, 30 aa, Artificial Sequence; identical to SEQ ID NO: 47
  Synthetic Construct

SEQ ID NO: 223 - protein, 30 aa, Artificial Sequence; identical to SEQ ID NO: 48
  Synthetic Construct

SEQ ID NO: 224 - protein, 30 aa, Artificial Sequence; identical to SEQ ID NO: 67
  Synthetic Construct

SEQ ID NO: 225 - protein, 30 aa, Artificial Sequence; identical to SEQ ID NO: 84
  Synthetic Construct

SEQ ID NO: 226 - protein, 30 aa, Artificial Sequence; identical to SEQ ID NO: 90
  Synthetic Construct

SEQ ID NO: 227 - protein, 30 aa, Artificial Sequence; identical to SEQ ID NO: 100
  Synthetic Construct

SEQ ID NO: 228 - protein, 30 aa, Artificial Sequence; identical to SEQ ID NO: 109
  Synthetic Construct

SEQ ID NO: 229 - protein, 30 aa, Artificial Sequence; identical to SEQ ID NO: 110
  Synthetic Construct

SEQ ID NO: 230 - protein, 30 aa, Artificial Sequence; identical to SEQ ID NO: 169
  Synthetic Construct

SEQ ID NO: 231 - protein, 30 aa, Artificial Sequence; identical to SEQ ID NO: 171
  Synthetic Construct

SEQ ID NO: 232 - protein, 30 aa, Artificial Sequence; identical to SEQ ID NO: 63
  Synthetic Construct

SEQ ID NO: 233 - protein, 30 aa, Artificial Sequence; identical to SEQ ID NO: 9
  Synthetic Construct

SEQ ID NO: 234 - protein, 30 aa, Artificial Sequence; identical to SEQ ID NO: 10
  Synthetic Construct

SEQ ID NO: 235 - protein, 30 aa, Artificial Sequence; identical to SEQ ID NO: 18
  Synthetic Construct

SEQ ID NO: 236 - protein, 30 aa, Artificial Sequence; identical to SEQ ID NO: 11
  Synthetic Construct

SEQ ID NO: 237 - protein, 30 aa, Artificial Sequence; identical to SEQ ID NO: 38
  Synthetic Construct

SEQ ID NO: 238 - protein, 30 aa, Artificial Sequence; identical to SEQ ID NO: 24
  Synthetic Construct

SEQ ID NO: 239 - protein, 30 aa, Artificial Sequence; identical to SEQ ID NO: 39
  Synthetic Construct

SEQ ID NO: 240 - protein, 30 aa, Artificial Sequence; identical to SEQ ID NO: 26
  Synthetic Construct

SEQ ID NO: 241 - protein, 30 aa, Artificial Sequence; identical to SEQ ID NO: 35
  Synthetic Construct

SEQ ID NO: 242 - protein, 30 aa, Artificial Sequence; identical to SEQ ID NO: 52
  Synthetic Construct

SEQ ID NO: 243 - protein, 30 aa, Artificial Sequence; identical to SEQ ID NO: 43
  Synthetic Construct

SEQ ID NO: 244 - protein, 30 aa, Artificial Sequence; identical to SEQ ID NO: 15
  Synthetic Construct

SEQ ID NO: 245 - protein, 30 aa, Artificial Sequence; identical to SEQ ID NO: 21
  Synthetic Construct

SEQ ID NO: 246 - protein, 30 aa, Artificial Sequence; identical to SEQ ID NO: 17
  Synthetic Construct

SEQ ID NO: 247 - protein, 30 aa, Artificial Sequence; identical to SEQ ID NO: 41
  Synthetic Construct

SEQ ID NO: 248 - protein, 30 aa, Artificial Sequence; identical to SEQ ID NO: 66
  Synthetic Construct

SEQ ID NO: 249 - protein, 30 aa, Artificial Sequence; identical to SEQ ID NO: 22
  Synthetic Construct

SEQ ID NO: 250 - protein, 30 aa, Artificial Sequence; identical to SEQ ID NO: 13
  Synthetic Construct

SEQ ID NO: 251 - protein, 30 aa, Artificial Sequence; identical to SEQ ID NO: 12
  Synthetic Construct

SEQ ID NO: 252 - protein, 30 aa, Artificial Sequence; identical to SEQ ID NO: 13
  Synthetic Construct

SEQ ID NO: 253 - protein, 30 aa, Artificial Sequence; identical to SEQ ID NO: 49
  Synthetic Construct

SEQ ID NO: 254 - protein, 30 aa, Artificial Sequence; identical to SEQ ID NO: 87
  Synthetic Construct

SEQ ID NO: 255 - protein, 30 aa, Artificial Sequence; identical to SEQ ID NO: 34
  Synthetic Construct

SEQ ID NO: 256 - protein, 30 aa, Artificial Sequence; identical to SEQ ID NO: 64
  Synthetic Construct

SEQ ID NO: 257 - protein, 30 aa, Artificial Sequence; identical to SEQ ID NO: 33
  Synthetic Construct

SEQ ID NO: 258 - protein, 30 aa, Artificial Sequence; identical to SEQ ID NO: 29
  Synthetic Construct

SEQ ID NO: 259 - protein, 30 aa, Artificial Sequence; identical to SEQ ID NO: 32
  Synthetic Construct

SEQ ID NO: 260 - protein, 30 aa, Artificial Sequence; identical to SEQ ID NO: 30
  Synthetic Construct

SEQ ID NO: 261 - protein, 30 aa, Artificial Sequence; identical to SEQ ID NO: 54
  Synthetic Construct

SEQ ID NO: 262 - protein, 30 aa, Artificial Sequence; identical to SEQ ID NO: 71
  Synthetic Construct

SEQ ID NO: 263 - protein, 30 aa, Artificial Sequence; identical to SEQ ID NO: 50
  Synthetic Construct

SEQ ID NO: 264 - protein, 30 aa, Artificial Sequence; identical to SEQ ID NO: 47
  Synthetic Construct

SEQ ID NO: 265 - protein, 30 aa, Artificial Sequence; identical to SEQ ID NO: 55
  Synthetic Construct

SEQ ID NO: 266 - protein, 30 aa, Artificial Sequence; identical to SEQ ID NO: 51
  Synthetic Construct

SEQ ID NO: 267 - protein, 30 aa, Artificial Sequence; identical to SEQ ID NO: 31
  Synthetic Construct

SEQ ID NO: 268 - protein, 30 aa, Artificial Sequence; identical to SEQ ID NO: 19
  Synthetic Construct

SEQ ID NO: 269 - protein, 30 aa, Artificial Sequence; identical to SEQ ID NO: 14
  Synthetic Construct

SEQ ID NO: 270 - protein, 30 aa, Artificial Sequence; identical to SEQ ID NO: 28
  Synthetic Construct

SEQ ID NO: 271 - protein, 30 aa, Artificial Sequence; identical to SEQ ID NO: 37
  Synthetic Construct

SEQ ID NO: 272 - protein, 30 aa, Artificial Sequence; identical to SEQ ID NO: 20
  Synthetic Construct

SEQ ID NO: 273 - protein, 30 aa, Artificial Sequence; identical to SEQ ID NO: 76
  Synthetic Construct

SEQ ID NO: 274 - protein, 30 aa, Artificial Sequence; identical to SEQ ID NO: 110
  Synthetic Construct

SEQ ID NO: 275 - protein, 30 aa, Artificial Sequence; identical to SEQ ID NO: 144
  Synthetic Construct

SEQ ID NO: 276 - protein, 30 aa, Artificial Sequence; identical to SEQ ID NO: 13
  Synthetic Construct

SEQ ID NO: 277 - protein, 30 aa, Artificial Sequence; identical to SEQ ID NO: 158
  Synthetic Construct

SEQ ID NO: 278 - protein, 30 aa, Artificial Sequence; identical to SEQ ID NO: 166
  Synthetic Construct

SEQ ID NO: 279 - protein, 30 aa, Artificial Sequence; identical to SEQ ID NO: 174
  Synthetic Construct

SEQ ID NO: 280 - protein, 30 aa, Artificial Sequence; identical to SEQ ID NO: 77
  Synthetic Construct

SEQ ID NO: 281 - protein, 30 aa, Artificial Sequence; identical to SEQ ID NO: 63
  Synthetic Construct

SEQ ID NO: 282 - protein, 30 aa, Artificial Sequence; identical to SEQ ID NO: 65
  Synthetic Construct

SEQ ID NO: 283 - protein, 30 aa, Artificial Sequence; identical to SEQ ID NO: 9
  Synthetic Construct

SEQ ID NO: 284 - protein, 30 aa, Artificial Sequence; identical to SEQ ID NO: 10
  Synthetic Construct

SEQ ID NO: 285 - protein, 30 aa, Artificial Sequence; identical to SEQ ID NO: 18
  Synthetic Construct

SEQ ID NO: 286 - protein, 30 aa, Artificial Sequence; identical to SEQ ID NO: 11
  Synthetic Construct

SEQ ID NO: 287 - protein, 30 aa, Artificial Sequence; identical to SEQ ID NO: 24
  Synthetic Construct

SEQ ID NO: 288 - protein, 30 aa, Artificial Sequence; identical to SEQ ID NO: 91
  Synthetic Construct

SEQ ID NO: 289 - protein, 30 aa, Artificial Sequence; identical to SEQ ID NO: 39
  Synthetic Construct

SEQ ID NO: 290 - protein, 30 aa, Artificial Sequence; identical to SEQ ID NO: 48
  Synthetic Construct

SEQ ID NO: 291 - protein, 30 aa, Artificial Sequence; identical to SEQ ID NO: 45
  Synthetic Construct

SEQ ID NO: 292 - protein, 30 aa, Artificial Sequence; identical to SEQ ID NO: 26
  Synthetic Construct

SEQ ID NO: 293 - protein, 30 aa, Artificial Sequence; identical to SEQ ID NO: 27
  Synthetic Construct

SEQ ID NO: 294 - protein, 30 aa, Artificial Sequence; identical to SEQ ID NO: 46
  Synthetic Construct

SEQ ID NO: 295 - protein, 30 aa, Artificial Sequence; identical to SEQ ID NO: 35
  Synthetic Construct

SEQ ID NO: 296 - protein, 30 aa, Artificial Sequence; identical to SEQ ID NO: 43
  Synthetic Construct

SEQ ID NO: 297 - protein, 30 aa, Artificial Sequence; identical to SEQ ID NO: 15
  Synthetic Construct

SEQ ID NO: 298 - protein, 30 aa, Artificial Sequence; identical to SEQ ID NO: 21
  Synthetic Construct

SEQ ID NO: 299 - protein, 30 aa, Artificial Sequence; identical to SEQ ID NO: 17
  Synthetic Construct

SEQ ID NO: 300 - protein, 30 aa, Artificial Sequence; identical to SEQ ID NO: 66
  Synthetic Construct

SEQ ID NO: 301 - protein, 30 aa, Artificial Sequence; identical to SEQ ID NO: 22
  Synthetic Construct

SEQ ID NO: 302 - protein, 30 aa, Artificial Sequence; identical to SEQ ID NO: 13
  Synthetic Construct

SEQ ID NO: 303 - protein, 30 aa, Artificial Sequence; identical to SEQ ID NO: 12
  Synthetic Construct

SEQ ID NO: 304 - protein, 30 aa, Artificial Sequence; identical to SEQ ID NO: 13
  Synthetic Construct

SEQ ID NO: 305 - protein, 30 aa, Artificial Sequence; identical to SEQ ID NO: 60
  Synthetic Construct

SEQ ID NO: 306 - protein, 30 aa, Artificial Sequence; identical to SEQ ID NO: 96
  Synthetic Construct

SEQ ID NO: 307 - protein, 30 aa, Artificial Sequence; identical to SEQ ID NO: 67
  Synthetic Construct

SEQ ID NO: 308 - protein, 30 aa, Artificial Sequence; identical to SEQ ID NO: 87
  Synthetic Construct

SEQ ID NO: 309 - protein, 30 aa, Artificial Sequence; identical to SEQ ID NO: 56
  Synthetic Construct

SEQ ID NO: 310 - protein, 30 aa, Artificial Sequence; identical to SEQ ID NO: 34
  Synthetic Construct

SEQ ID NO: 311 - protein, 30 aa, Artificial Sequence; identical to SEQ ID NO: 16
  Synthetic Construct

SEQ ID NO: 312 - protein, 30 aa, Artificial Sequence; identical to SEQ ID NO: 64
  Synthetic Construct

SEQ ID NO: 313 - protein, 30 aa, Artificial Sequence; identical to SEQ ID NO: 33
  Synthetic Construct

SEQ ID NO: 314 - protein, 30 aa, Artificial Sequence; identical to SEQ ID NO: 32
  Synthetic Construct

SEQ ID NO: 315 - protein, 30 aa, Artificial Sequence; identical to SEQ ID NO: 30
  Synthetic Construct

SEQ ID NO: 316 - protein, 30 aa, Artificial Sequence; identical to SEQ ID NO: 100
  Synthetic Construct

SEQ ID NO: 317 - protein, 30 aa, Artificial Sequence; identical to SEQ ID NO: 54
  Synthetic Construct

SEQ ID NO: 318 - protein, 30 aa, Artificial Sequence; identical to SEQ ID NO: 72
  Synthetic Construct

SEQ ID NO: 319 - protein, 30 aa, Artificial Sequence; identical to SEQ ID NO: 47
  Synthetic Construct

SEQ ID NO: 320 - protein, 30 aa, Artificial Sequence; identical to SEQ ID NO: 55
  Synthetic Construct

SEQ ID NO: 321 - protein, 30 aa, Artificial Sequence; identical to SEQ ID NO: 51
  Synthetic Construct

SEQ ID NO: 322 - protein, 30 aa, Artificial Sequence; identical to SEQ ID NO: 31
  Synthetic Construct

SEQ ID NO: 323 - protein, 30 aa, Artificial Sequence; identical to SEQ ID NO: 14
  Synthetic Construct

SEQ ID NO: 324 - protein, 30 aa, Artificial Sequence; identical to SEQ ID NO: 36
  Synthetic Construct

SEQ ID NO: 325 - protein, 30 aa, Artificial Sequence; identical to SEQ ID NO: 28
  Synthetic Construct

SEQ ID NO: 326 - protein, 30 aa, Artificial Sequence; identical to SEQ ID NO: 37
  Synthetic Construct

SEQ ID NO: 327 - protein, 30 aa, Artificial Sequence; identical to SEQ ID NO: 44
  Synthetic Construct

SEQ ID NO: 328 - protein, 30 aa, Artificial Sequence; identical to SEQ ID NO: 20
  Synthetic Construct

SEQ ID NO: 329 - protein, 30 aa, Artificial Sequence; identical to SEQ ID NO: 109
  Synthetic Construct

SEQ ID NO: 330 - protein, 30 aa, Artificial Sequence; identical to SEQ ID NO: 110
  Synthetic Construct

SEQ ID NO: 331 - protein, 30 aa, Artificial Sequence; identical to SEQ ID NO: 122
  Synthetic Construct

SEQ ID NO: 332 - protein, 30 aa, Artificial Sequence; identical to SEQ ID NO: 130
  Synthetic Construct

SEQ ID NO: 333 - protein, 30 aa, Artificial Sequence; identical to SEQ ID NO: 162
  Synthetic Construct

SEQ ID NO: 334 - protein, 30 aa, Artificial Sequence; identical to SEQ ID NO: 169
  Synthetic Construct

SEQ ID NO: 335 - protein, 30 aa, Artificial Sequence; identical to SEQ ID NO: 171
  Synthetic Construct

SEQ ID NO: 336 - protein, 30 aa, Artificial Sequence; identical to SEQ ID NO: 173
  Synthetic Construct

SEQ ID NO: 337 - protein, 30 aa, Artificial Sequence; identical to SEQ ID NO: 175
  Synthetic Construct

SEQ ID NO: 338 - protein, 30 aa, Artificial Sequence; identical to SEQ ID NO: 187
  Synthetic Construct

SEQ ID NO: 339 - protein, 5 aa, Artificial Sequence
  Synthetic Construct
GSNKS

SEQ ID NO: 340 - protein, 10 aa, Artificial Sequence
  Synthetic Construct
GSNKSKPKDA

SEQ ID NO: 341 - protein, 7 aa, Artificial Sequence
  Synthetic Construct
PKKKRKV

SEQ ID NO: 342 - protein, 20 aa, Artificial Sequence
  Synthetic Construct
AVKRPAATKKAGQAKKKKLD

SEQ ID NO: 343 - protein, 25 aa, Artificial Sequence
  Synthetic Construct
MSRRRKANPTKLSENAKKLAKEVEN

SEQ ID NO: 344 - protein, 9 aa, Artificial Sequence
  Synthetic Construct
PAAKRVKLD

SEQ ID NO: 345 - protein, 9 aa, Artificial Sequence
  Synthetic Construct
KLKIKRPVK

SEQ ID NO: 346 - DNA, 21 bp, Artificial Sequence
  Synthetic Construct
cccaagaaaaaacgcaaggtg

SEQ ID NO: 347 - DNA, 21 bp, Artificial Sequence
  Synthetic Construct
cctaagaaaaagcggaaagtg

SEQ ID NO: 348 - DNA, 30 bp, Artificial Sequence
  Synthetic Construct
gagcagaaactcatctcagaagaggatctg

SEQ ID NO: 349 - DNA, 24 bp, Artificial Sequence
  Synthetic Construct
gattacaaggatgacgacgataag

SEQ ID NO: 350 - DNA, 13425 bp, Artificial Sequence
  Synthetic Construct
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagaaataatagcaacagacatacaaactaaagaattacaaaaacaaattacaaaaattcaaaattttcgggtttattacagggacagcagagatccagtttggttaatccgctagctctagaggatctgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatccactggaccaggcagcagcgtcagaagacttttttggaacgtctcgttttagagctagaaatagcaagttaaaataaggctagtccgttatcaacttgaaaaagtggcaccgagtcggtgctttttttggtgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggaaccggtaccgaggagatctgccgccgcgatcgccatgggcagcaacaagagcaagcccaaggataagaaatactcaataggactggatattggcacaaatagcgtcggatgggctgtgatcactgatgaatataaggttccttctaaaaagttcaaggttctgggaaatacagaccgccacagtatcaaaaaaaatcttataggggctcttctgtttgacagtggagagacagccgaagctactagactcaaacggacagctaggagaaggtatacaagacggaagaataggatttgttatctccaggagattttttcaaatgagatggccaaagtggatgatagtttctttcatagacttgaagagtcttttttggtggaagaagacaagaagcatgaaagacatcctatttttggaaatatagtggatgaagttgcttatcacgagaaatatccaactatctatcatctgagaaaaaaattggtggattctactgataaagccgatttgcgcctgatctatttggccctggcccacatgattaagtttagaggtcattttttgattgagggcgatctgaatcctgataatagtgatgtggacaaactgtttatccagttggtgcaaacctacaatcaactgtttgaagaaaaccctattaacgcaagtggagtggatgctaaagccattctttctgcaagattgagtaaatcaagaagactggaaaatctcattgctcagctccccggtgagaagaaaaatggcctgtttgggaatctcattgctttgtcattgggtttgacccctaattttaaatcaaattttgatttggcagaagatgctaaactccagctttcaaaagatacttacgatgatgatctggataatctgttggctcaaattggagatcaatatgctgatttgtttttggcagctaagaatctgtcagatgctattctgctttcagacatcctgagagtgaatactgaaataactaaggctcccctgtcagcttcaatgattaaacgctacgatgaacatcatcaagacttgactcttctgaaagccctggttagacaacaacttccagaaaagtataaagaaatcttttttgatcaatcaaaaaacggatatgcaggttatattgatggcggcgcaagccaagaagaattttataaatttatcaaaccaattctggaaaaaatggatggtactgaggaactgttggtgaaactgaatagagaagatttgctgcgcaagcaacggacctttgacaacggctctattccccatcaaattcacttgggtgagctgcatgctattttgagaagacaagaagacttttatccatttctgaaagacaatagagagaagattgaaaaaatcttgacttttaggattccttattatgttggtccattggccagaggcaatagtaggtttgcatggatgactcggaagtctgaagaaacaattaccccatggaattttgaagaagttgtcgataaaggtgcttcagctcaatcatttattgaacgcatgacaaactttgataaaaatcttccaaatgaaaaagtgctgccaaaacatagtttgctttatgagtattttaccgtttataacgaattgacaaaggtcaaatatgttactgaaggaatgagaaaaccagcatttctttcaggtgaacagaagaaagccattgttgatctgctcttcaaaacaaataggaaagtgaccgttaagcaactgaaagaagattatttcaaaaaaatagaatgttttgatagtgttgaaatttcaggagttgaagatagatttaatgcttcactgggtacataccatgatttgctgaaaattattaaagataaagattttttggataatgaagaaaatgaagacatcctggaggatattgttctgacattgaccctgtttgaagatagggagatgattgaggaaagacttaaaacatacgctcacctctttgatgataaggtgatgaaacagcttaaaagacgcagatatactggttggggaaggttgtccagaaaattgattaatggtattagggataagcaatctggcaaaacaatactggattttttgaaatcagatggttttgccaatcgcaattttatgcagctcatccatgatgatagtttgacatttaaagaagacatccaaaaagcacaagtgtctggacaaggcgatagtctgcatgaacatattgcaaatctggctggtagccctgctattaaaaaaggtattctccagactgtgaaagttgttgatgaattggtcaaagtgatggggcggcataagccagaaaatatcgttattgaaatggcaagagaaaatcagacaactcaaaagggccagaaaaattccagagagaggatgaaaagaatcgaagaaggtatcaaagaactgggaagtcagattcttaaagagcatcctgttgaaaatactcaattgcaaaatgaaaagctctatctctattatctccaaaatggaagagatatgtatgtggaccaagaactggatattaataggctgagtgattatgatgtcgatcacattgttccacaaagtttccttaaagacgattcaatagacaataaggtcctgaccaggtctgataaaaatagaggtaaatccgataacgttccaagtgaagaagtggtcaaaaagatgaaaaactattggagacaacttctgaacgccaagctgatcactcaaaggaagtttgataatctgaccaaagctgaaagaggaggtttgagtgaacttgataaagctggttttatcaaacgccaattggttgaaactcgccaaatcactaagcatgtggcacaaattttggatagtcgcatgaatactaaatacgatgaaaatgataaacttattagagaggttaaagtgattaccctgaaatctaaactggtttctgacttcagaaaagatttccaattctataaagtgagagagattaacaattaccatcatgcccatgatgcctatctgaatgccgtcgttggaactgctttgattaagaaatatccaaaacttgaaagcgagtttgtctatggtgattataaagtttatgatgttaggaaaatgattgctaagtctgagcaagaaataggcaaagcaaccgcaaagtatttcttttactctaatatcatgaacttcttcaaaacagaaattacacttgcaaatggagagattcgcaaacgccctctgatcgaaactaatggggaaactggagaaattgtctgggataaagggagagattttgccacagtgcgcaaagtgttgtccatgccccaagtcaatatcgtcaagaaaacagaagtgcagacaggcggattctctaaggagtcaattctgccaaaaagaaattccgacaagctgattgctaggaaaaaagactgggacccaaaaaaatatggtggttttgatagtccaaccgtggcttattcagtcctggtggttgctaaggtggaaaaagggaaatccaagaagctgaaatccgttaaagagctgctggggatcacaattatggaaagaagttcctttgaaaaaaatcccattgactttctggaagctaaaggatataaggaagttaaaaaagacctgatcattaaactgcctaaatatagtctttttgagctggaaaacggtaggaaacggatgctggctagtgccggagaactgcaaaaaggaaatgagctggctctgccaagcaaatatgtgaattttctgtatctggctagtcattatgaaaagttgaagggtagtccagaagataacgaacaaaaacaattgtttgtggagcagcataagcattatctggatgagattattgagcaaatcagtgaattttctaagagagttattctggcagatgccaatctggataaagttcttagtgcatataacaaacatagagacaaaccaataagagaacaagcagaaaatatcattcatctgtttaccttgaccaatcttggagcacccgctgcttttaaatactttgatacaacaattgataggaaaagatatacctctacaaaagaagttctggatgccactcttatccatcaatccatcactggtctttatgaaacacgcattgatttgagtcagctgggaggtgaccccaagaaaaaacgcaaggtggaagatcctaagaaaaagcggaaagtggacacgcgtacgcggccgctcgagcagaaactcatctcagaagaggatctggcagcaaatgatatcctggattacaaggatgacgacgataaggtttaacttaattaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgcccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtccttcggccctcaatccaagcggaccttccttcccgcggcctgctgccggctctgcgggcctcttccgcgtctttcgccttcgccctcagacgagtcggatctccctttgggcgctccccgcatcgatgtcgacctcgagaccggccgaactcgaagacctagaaaaaacattggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 351 - protein, 6 aa, Artificial Sequence
  Synthetic Construct
GSNKSK

SEQ ID NO: 352 - protein, 6 aa, Artificial Sequence
  Synthetic Construct
ASNKSK

SEQ ID NO: 353 - protein, 6 aa, Artificial Sequence
  Synthetic Construct
GCNKCK

SEQ ID NO: 354 - protein, 6 aa, Artificial Sequence
  Synthetic Construct
GCVQCK

SEQ ID NO: 355 - protein, 6 aa, Artificial Sequence
  Synthetic Construct
ACVQCK

SEQ ID NO: 356 - protein, 6 aa, Artificial Sequence
  Synthetic Construct
GSVQSK

SEQ ID NO: 357 - protein, 8 aa, Artificial Sequence
  Synthetic Construct
GCIKSKEN

SEQ ID NO: 358 - protein, 8 aa, Artificial Sequence
  Synthetic Construct
GCVQCKDK

SEQ ID NO: 359 - protein, 8 aa, Artificial Sequence
  Synthetic Construct
GAQFSKTA

SEQ ID NO: 360 - protein, 8 aa, Artificial Sequence
  Synthetic Construct
GSQSSKAP

SEQ ID NO: 361 - protein, 8 aa, Artificial Sequence
  Synthetic Construct
GNAQERPS

SEQ ID NO: 362 - protein, 8 aa, Artificial Sequence
  Synthetic Construct
GRKSSKAK

SEQ ID NO: 363 - protein, 8 aa, Artificial Sequence
  Synthetic Construct
GQSQSGGH

SEQ ID NO: 364 - protein, 8 aa, Artificial Sequence
  Synthetic Construct
GAKQSGPA

SEQ ID NO: 365 - protein, 8 aa, Artificial Sequence
  Synthetic Construct
GNCLKSPT

SEQ ID NO: 366 - protein, 8 aa, Artificial Sequence
  Synthetic Construct
GSNKSKPK

SEQ ID NO: 367 - protein, 8 aa, Artificial Sequence
  Synthetic Construct
GCIKSKGK

SEQ ID NO: 368 - protein, 8 aa, Artificial Sequence
  Synthetic Construct
GSENSALK

SEQ ID NO: 369 - protein, 8 aa, Artificial Sequence
  Synthetic Construct
GSCCSCPD

SEQ ID NO: 370 - protein, 8 aa, Artificial Sequence
  Synthetic Construct
GCFFSKRR

SEQ ID NO: 371 - protein, 8 aa, Artificial Sequence
  Synthetic Construct
GGLFSRWR

SEQ ID NO: 372 - protein, 8 aa, Artificial Sequence
  Synthetic Construct
GALVIRGI

SEQ ID NO: 373 - protein, 8 aa, Artificial Sequence
  Synthetic Construct
GQKASQQL

SEQ ID NO: 374 - protein, 8 aa, Artificial Sequence
  Synthetic Construct
GCRQSSEE

SEQ ID NO: 375 - protein, 8 aa, Artificial Sequence
  Synthetic Construct
GETMSKRL

SEQ ID NO: 376 - protein, 8 aa, Artificial Sequence
  Synthetic Construct
GSRVSRED

SEQ ID NO: 377 - protein, 8 aa, Artificial Sequence
  Synthetic Construct
GLLDRLSV

SEQ ID NO: 378 - protein, 8 aa, Artificial Sequence
  Synthetic Construct
GKVLSKIF

SEQ ID NO: 379 - protein, 8 aa, Artificial Sequence
  Synthetic Construct
GLLTILKK

SEQ ID NO: 380 - protein, 8 aa, Artificial Sequence
  Synthetic Construct
GAHLVRRY

SEQ ID NO: 381 - protein, 8 aa, Artificial Sequence
  Synthetic Construct
GRESRHYR

SEQ ID NO: 382 - protein, 8 aa, Artificial Sequence
  Synthetic Construct
ASNKSKPK

SEQ ID NO: 383 - DNA, 83 bp, Artificial Sequence
  Synthetic Construct
catagatctgccgccgcgatcgccatgggcagcaacaagagcaagcccaaggataagaaatactcaataggactggatattgg

SEQ ID NO: 384 - DNA, 52 bp, Artificial Sequence
  Synthetic Construct
catagatctgccgccgcgatcgccatggccagcaacaagagcaagcccaagg

SEQ ID NO: 385 - DNA, 52 bp, Artificial Sequence
  Synthetic Construct
catagatctgccgccgcgatcgccatgggctgcaacaagagcaagcccaagg

SEQ ID NO: 386 - DNA, 52 bp, Artificial Sequence
  Synthetic Construct
catagatctgccgccgcgatcgccatgggcagcaacaagtgcaagcccaagg

SEQ ID NO: 387 - DNA, 52 bp, Artificial Sequence
  Synthetic Construct
catagatctgccgccgcgatcgccatgggctgcaacaagtgcaagcccaagg

SEQ ID NO: 388 - DNA, 26 bp, Artificial Sequence
  Synthetic Construct
catgtataccttctcctagctgtccg

SEQ ID NO: 389 - DNA, 26 bp, Artificial Sequence
  Synthetic Construct
gatcggggcgaggagctgttcaccgg

SEQ ID NO: 390 - DNA, 26 bp, Artificial Sequence
  Synthetic Construct
aaaaccggtgaacagctcctcgcccc

SEQ ID NO: 391 - DNA, 26 bp, Artificial Sequence
  Synthetic Construct
gatcggagctggacggcgacgtaaag

SEQ ID NO: 392 - DNA, 26 bp, Artificial Sequence
  Synthetic Construct
aaaactttacgtcgccgtccagctcc

SEQ ID NO: 393 - DNA, 26 bp, Artificial Sequence
  Synthetic Construct
gatcgggccacaagttcagcgtgtcg

SEQ ID NO: 394 - DNA, 26 bp, Artificial Sequence
  Synthetic Construct
aaaacgacacgctgaacttgtggccc

SEQ ID NO: 395 - DNA, 26 bp, Artificial Sequence
  Synthetic Construct
gatcgacaactttaccgaccgcgccg

SEQ ID NO: 396 - DNA, 26 bp, Artificial Sequence
  Synthetic Construct
aaaacggcgcggtcggtaaagttgtc

SEQ ID NO: 397 - DNA, 19 bp, Artificial Sequence
  Synthetic Construct
aaattgcttctggtggcgc

SEQ ID NO: 398 - DNA, 20 bp, Artificial Sequence
  Synthetic Construct
cgtcttcgtcccagtaagct

SEQ ID NO: 399 - DNA, 24 bp, Artificial Sequence
  Synthetic Construct
ggactatcatatgcttaccgtaac

SEQ ID NO: 400 - DNA, 26 bp, Artificial Sequence
  Synthetic Construct
catgtataccttctcctagctgtccg

(19) United States
(12) Patent Application Publication
(10) Pub. No.: US 2022/0195455 A1
(43) Pub. Date: Jun. 23, 2022

(54) DELIVERY OF CRISPR/MCAS9 THROUGH EXTRACELLULAR VESICLES FOR GENOME EDITING

(71) Applicant: University of Georgia Research Foundation, Inc., Athens, GA (US)

(72) Inventor: Houjian Cai, Athens, GA (US)

(21) Appl. No.: 17/441,571

(22) PCT Filed: Apr. 2, 2020
(86) PCT No.: PCT/US2020/026321

§ 371 Date: Sep. 21, 2021

Related U.S. Application Data
(60) Provisional application No. 62/828,776, filed on Apr. 3, 2019.

(51) Int. Cl. C12N 15/85; C12N 9/22; C12N 15/62; C12N 15/90; C12N 15/11

(52) U.S. Cl. CPC C12N 15/85; C12N 9/22; C12N 2310/20; C12N 15/907; C12N 15/11; C12N 15/62

(57) ABSTRACT

Disclosed herein is a fusion protein for gene editing, comprising a Cas9 domain that is configured to be encapsulated into exosomes and to localize to the nucleus of recipient cells. Also disclosed are recombinant polynucleotides that comprise a nucleic acid sequence encoding the disclosed Cas9 fusion protein. Also disclosed are cells comprising the disclosed polynucleotides. Also disclosed are methods of making a gene editing composition that involve culturing the disclosed cells under conditions suitable to produce extracellular vesicles encapsulating the guide RNA and fusion protein. Also disclosed are gene editing compositions that involve extracellular vesicles encapsulating the disclosed Cas9 fusion proteins and guide RNA. Finally, also disclosed herein are methods for editing a gene in a cell that involves contact the cell with the herein disclosed gene editing compositions.

### CROSS-REFERENCE TO RELATED APPLICATIONS

This application claims benefit of U.S. Provisional Application No. 62/828,776, filed Apr. 3, 2019, which is hereby incorporated herein by reference in its entirety.

### SEQUENCE LISTING

This application contains a sequence listing filed in electronic form as an ASCII.txt file entitled “222102_2940_Sequence_Listing_ST25” created on Mar. 20, 2020. The content of the sequence listing is incorporated herein in its entirety.

### BACKGROUND

The CRISPR-Cas9 genome-editing system is a part of the adaptive immune system in archaea and bacteria to defend against invasive nucleic acids from phages and plasmids. The single guide RNA (sgRNA) of the system recognizes its target sequence in the genome, and the Cas9 nuclease of the system acts as a pair of scissors to cleave the double strands of DNA. Since its discovery, CRISPR-Cas9 has become the most robust platform for genome engineering in eukaryotic cells. Recently, the CRISPR-Cas9 system has triggered enormous interest in therapeutic applications. CRISPR-Cas9 can be applied to correct disease-causing gene mutations or engineer T cells for cancer immunotherapy. The first clinical trial using the CRISPR-Cas9 technology was conducted in 2016. Despite the great promise of the CRISPR-Cas9 technology, several challenges remain to be tackled before its successful applications for human patients. The greatest challenge is the safe and efficient delivery of the CRISPR-Cas9 genome-editing system to target cells in human body.

### SUMMARY

Disclosed herein is a fusion protein for gene editing, comprising a Cas9 domain that is configured to be encapsulated into extracellular vesicles (EVS) and to localize to the nucleus of recipient cells. The fusion should possess the following criteria: 1) it should be encapsulated into EVs; and 2) it should be taken into the recipient cells, and be localized into the nucleus for genome editing. The fusion protein can therefore contain a myristoylation domain and possess a positive charge in the N-terminus of the fusion protein, which allows encapsulation of the protein in EVs. As disclosed herein, palmitoylation of the peptide can significantly inhibit encapsulation and/or nucleus localization. Therefore, in some embodiments, the disclosed fusion protein contains a myristoylation motif, but does not contain a palmitoylation motif.

Therefore, disclosed herein is a fusion protein, comprising a myristoylation domain, a Cas9 domain, and a nuclear localization signal (NLS), wherein the myristoylation domain is configured to be myristoylated during protein translation. In some embodiments, the fusion protein comprises a myristoylation domain that possesses a myristoylation motif followed with positively charged amino acids but does not contain a palmitoylation motif.

The disclosed system can be used to encapsulate any protein or peptide into extracellular vesicles. Therefore, disclosed herein is a fusion protein, comprising a myristoylation domain, a protein domain, and a nuclear localization signal (NLS), wherein the myristoylation domain is configured to be myristoylated during protein translation. The protein domain can be any protein or peptide for which cell delivery is desired. In some embodiments, the protein domain is an enzyme, ligand, or receptor. In some embodiments, the fusion protein comprises a myristoylation domain that possesses a myristoylation motif followed with positively charged amino acids but does not contain a palmitoylation motif.

Myristoylation is a lipidation modification where a myristoyl group, derived from myristic acid, is covalently attached by an amide bond to the alpha-amino group of an N-terminal glycine residue. Briefly, proteins that will become myristoylated begin with a consensus sequence Met-Gly-X-X-X-Ser/Thr (SEQ ID NO:3). The start Met is cotranslationally, proteolytically removed and the myristate is added to the exposed N-terminal glycine via a stable amide bond. As used herein, “palmitoylation” refers the covalent attachment of fatty acids, such as palmitic acid, to cysteine. Therefore, in some embodiments, the myristoylation domain of the disclosed fusion protein does not comprises a cysteine residue. Therefore, in some embodiments, the myristoylation domain comprises the amino acid sequence G-X-X-X-S/T (SEQ ID NO:1), wherein X is any amino acid other than Cys.

Also disclosed herein is a recombinant polynucleotide that comprises a nucleic acid sequence encoding a guide RNA operably linked to a first expression control sequence, and a nucleic acid sequence encoding the disclosed Cas9 fusion protein operably linked to a second expression control sequence.

Also disclosed herein is any types of cells being transduced with the disclosed polynucleotide. In some embodiments, the cell is any types of cell capable of producing extracellular vesicles, such as exosomes. Also disclosed is a method of making a gene editing composition, comprising culturing the disclosed cell under conditions suitable to produce extracellular vesicles encapsulating the guide RNA and fusion protein.

Also disclosed is a gene editing composition, comprising an extracellular vesicle encapsulating the disclosed Cas9 fusion protein and a guide RNA. Finally, also disclosed herein is a method for editing a gene in a cell that involves contact the cell with the herein disclosed gene editing composition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show the appearance frequency of myristoylated proteins is elevated in extracellular vesicles (EVs). FIG. 1A shows 182 potentially myristoylated proteins, which contain a glycine at site 2, were identified in the mammalian genome. Given about a total of 20,000 proteins in a mammalian cell, the frequency of myristoylated proteins accounts for about 0.9% of the mammalian genome. The number of myristoylated proteins (red, numerator) and total proteins (black, denominator) in EVs detected through proteomics is analyzed from four studies including one study for 60 cancer cell lines (Table 1-2) and three other studies for normal tissues (thymus, breast milk, and urine) (Table 3-5) (35-40). FIG. 1B shows the appearance frequency of myristoylated proteins in EVs in 60 individual cancer cell lines (35). The red line represents 0.9% of myristoylated proteins in the mammalian genome. FIG. 1C shows prostate cancer cells including DU145, PC3, 22Rv1 and LNCaP cells were cultured in medium containing 10% EVs/exosome-free FBS for 24 h. EVs were isolated from the conditioned medium by sequential centrifugation. Expression levels of Src kinase, AR, calnexin, GAPDH and CD9 (an exosomal protein marker) in extracellular vesicles (EVs) and total cell lysates (TCL) were analyzed by Western blot. The same amount of protein (10 μg) from the EVs or TCL were loaded. Src kinase was expressed in EVs of all tested cell lines. The ratio of Src protein level in EVs relative to that in TCL was calculated. The ratio in DU145 cells was significantly higher than that in other three cell lines. Data were expressed as mean±SEM, * p<0.05; ** p<0.01; *** p<0.001.

FIGS. 2A to 2C show loss of myristoylation inhibits the encapsulation of Src kinase into EVs. FIG. 2A is a schematic diagram of Src(WT) (GSNKSK, SEQ ID NO:352) and Src(G2A) (ASNKSK, SEQ ID NO:353) mutant. FIG. 2B shows DU145, NIH3T3, and SYF1(Src^{-/-}Yes^{-/-}Fyn^{-/-}) cells transduced with Src(WT) or Src(G2A) by lentiviral infection. The transfected cells were grown in exosome-free FBS medium and EVs were isolated from the conditioned medium. Expression levels of Src, Calnexin, GAPDH, and CD9 in extracellular vesicles (EVs) and total cell lysate (TCL) of the transduced cells were analyzed by Western blot. Ten μg of protein from EVs or TCL were loaded. Src protein levels were quantified by Image J software. The ratio of Src levels in EVs relative to TCL is shown. Data were expressed as mean±SEM, ** p<0.01; *** p<0.001. FIG. 2C shows DU145 cells transduced with control vector, Src(WT), or Src(G2A) by lentiviral infection. The transduced cells were grown in EVs/exosome-free FBS medium with (Lane 4-6 and 10-12) or without (Lane 1-3 and 7-9) 50 μM myristic acid-azide (an analog of myristic acid). The myristoylated proteins from either EVs or TCL were detected using Click chemistry. Ten μg of protein from EVs or TCL were loaded. Levels of Src, calnexin, GAPDH, and CD9 were measured by Western blot.

FIGS. 3A to 3C show activated Src kinase promotes its encapsulation into EVs. FIG. 3A is a schematic diagram of Src(Y529F) (GSNKSK, SEQ ID NO: 352) and Src(Y529F/G2A) (ASNKSK, SEQ ID NO:353) constructs. FIGS. 3B-3C show DU145 and SYF1 cells transduced with vector control, Src(WT), Src(G2A), Src(Y529F), or Src(Y529F/G2A) by lentiviral infection. EVs were isolated from conditioned medium by sequential ultracentrifugation. Expression levels of Src, calnexin, GAPDH, and CD9 in extracellular vesicles (EVs) and total cell lysates (TCL) derived from DU145 (FIG. 3B) and SYF1 (FIG. 3C) cells analyzed by Western blotting. Ten μg of protein from EVs or TCL were loaded. High exposure time shows low expression levels of Src kinase in EVs from SYF1 cells expressing Src(Y529F/G2A) in (FIG. 3C). Coomassie staining was used to show equivalent loading of samples. The Src expression level was quantified by Image J software. Data are expressed as mean±SEM, * p<0.05; ** p<0.01; *** p<0.001.

FIGS. 4A to 4C show myristoylation and palmitoylation regulate the encapsulation of Src family kinase proteins into EVs. FIG. 4A is a schematic diagram of Src(WT) (GSNKSK, SEQ ID NO:352), Src(G2A) (ASNKSK, SEQ ID NO:353), Src(S3C/S6C) (GCNKCK, SEQ ID NO:354), Fyn(WT) (GCVQCK, SEQ ID NO:355), Fyn(G2A) (ACVQCK, SEQ ID NO:356) and Fyn(C3S/C6S) (GSVQSK, SEQ ID NO:357) mutants. Src(G2A) and Fyn(G2A) mutants lead to loss of myristoylation. Src(S3C/S6C) results in the gain of palmitoylation, and Fyn(C3S/C6S) leads to loss of palmitoylation. FIGS. 4B to 4C show DU145 cells were transduced with Src(WT), Src(G2A), and Src(S3C/S6C) (FIG. 4B), or transduced with Fyn(WT), Fyn(G2A), and Fyn(C3S/C6S) (FIG. 4C) by lentiviral infection. The transduced cells were grown in EVs/exosome-free medium for 24 h and EVs were isolated from the conditioned medium. Ten μg of protein from extracellular vesicles (EVs) or total cell lysates (TCL) were loaded. Expression levels of Src or Fyn, Calnexin, GAPDH, and CD9 in Exo or TCL were analyzed by immunoblotting. The Src protein level was quantified by Image J. The ratio of Src or Fyn protein level in EVs relative to that in TCL was calculated. Data are expressed as mean±SEM. * p<0.05; **** p<0.0001; NS: Not significant.

FIGS. 5A to 5D show myristoylation facilitates the encapsulation of Src kinase into the plasma EVs. DU145 cells were transduced with control vector, Src(Y529F), or Src(Y529F/G2A) by lentiviral infection. The transduced DU145 cells (1×10⁴ cells/graft) were mixed with collagen and implanted sub-renally in SCID mice (3 months-old, n=3 per group). After 5 weeks, the mice were sacrificed, xenografts were harvested, and EVs were extracted from the blood plasma using the Exoquick kit. FIG. 5A shows the size, zeta potential, and particle number of EVs were measured by nanoparticle tracking analysis using the Particle Metrix Analyzer. FIGS. 5B to 5C are images (with the kidney) and weight of xenografts. FIG. 5D show expression levels of Src kinase, non-pSrc(Y529) (for detection of activated Src), and TSG101 (a marker of exosomes) in the plasma EVs were examined by immunoblotting. Coomassie staining was used to show equivalent loading of samples. Three experimental repeats (1 to 3) were shown. Data are expressed as mean±SEM. NS: Not significant. **: p<0.01

FIGS. 6A to 6D show detection of Src kinase in the plasma EVs depends on the myristoylation status of Src-induced xenograft tumors. DU145 cells expressing control vector (1.5×10⁵ cells/graft), Src(Y529F/G2A) (1.5×10⁵ cells/graft) or Src(Y529F) (1.5×10⁴ cells/graft) were implanted sub-renally into SCID mice. After 4 weeks, the mice were sacrificed and xenograft tumors and the plasma were harvested. FIG. 5A shows the size, zeta potential, and the particle number of the plasma EVs were analyzed. FIGS. 5B and 5C show the image (with the kidney) and weight of the xenograft tumors. FIG. 5D shows levels of Src, non-pSrc(Y529), TSG101 and flotillin-1 (protein markers of EVs) in the plasma EVs were determined by Western blotting. 50 μg of EVs protein was loaded. The Coomassie Blue staining was used to reflect the loading of the total amount protein. Three repeats (1 to 3) of each experimental group are shown. Data are expressed as mean±SEM. ***: p<0.01; NS: Not significant.

FIGS. 7A to 7C shows TSG101 levels, but not cholesterol levels, regulate the encapsulation of Src kinase into EVs. FIG. 7A shows PC3 or DU145 cells treated with Filipin III (0, 0.25, 0.5, and 1 μM) for 24 h. The depletion of cholesterol was visualized. Levels of Src, Calnexin, GAPDH, and CD9 in extracellular vesicles (EVs) and the total cell lysate (TCL) were analyzed by immunoblotting. FIGS. 7B to 7C show 22Rv1 and PC3 cells transfected with shRNA-control, shRNA-TSG101-1, or shRNA-TSG101-2 by lentiviral infection. The transduced 22Rv1 and PC3 cells were incubated with 10% EVs/exosome-free FBS for 48 h. EVs were isolated from the conditioned culture medium. Ten μg of EVs or TCL were loaded as determined by the DC protein assay. Levels of TSG101, Src, Calnexin, GAPDH, and CD9 were analyzed by Western blot. The ratio of Src levels in EVs to that in TCL in 22Rv1 (FIG. 7B) and PC3 cells (FIG. 7C) were calculated. The Coomassie Blue staining was used to reflect the loading of the total amount protein. Data are expressed as mean±SEM. *: p<0.05; **: p<0.01; ***: p<0.001; NS: Not significant.

FIG. 8 shows lipid acylation regulates Src family kinases to be encapsulated into EVs. Panel A shows myristoylation of Src kinase mediates its association with the cell membrane and the activation of kinase activity. The activated Src kinase presumably promotes the assembly of syntenin-syndecan and its interaction with the protein complex in the formation of multi-vesicular bodies from the cell membrane. Src encapsulation into EVs is mediated through ESCRT pathway. For example, TSG101, an essential element of ESCRT pathway, regulates Src encapsulation process. Panel B shows loss of myristoylation in Src(G2A) or Fyn(G2A) mutants inhibits its membrane association, thereby suppressing the formation of syntenin-syndecan and encapsulation into EVs. Panel C shows Fyn kinase or the gain of palmitoylation in Src(S3C/S6C) mutant localizes the protein in the lipid raft region of the cell membrane, which might similarly weaken the assembly of syntenin-syndecan interaction, subsequently its encapsulation into EVs.

FIGS. 9A to 9C shows the size, zeta potential, and particle concentration of EVs in the tested cells. Prostate cancer cells including DU145, PC3, 22Rv1 and LNCaP cells were cultured in the ATCC recommended medium containing 10% exosome-free FBS for 24 h. EVs were isolated from the conditioned medium by the sequential ultracentrifugation method. The average size and the size distribution (FIG. 9A), zeta potential (FIG. 9B), and particle concentration of EVs (FIG. 9C) were measured by nanoparticle tracking analysis using the Particle Metrix Analyzer. DU145 cells produced a significantly higher number of EVs than three other prostate cancer cells. Data are expressed as mean±SEM. * p<0.05; ** p<0.01; *** p<0.001. NS: not significant.

FIG. 10 shows loss of myristoylation decreases the encapsulation of Src kinase into EVs in 22Rv1 cells. 22Rv1 cells were transduced with Src(WT) or Src(G2A) by lentiviral infection. The transduced cells were grown in exosome-free FBS medium. EVs were collected from the conditioned cell culture medium. Expression levels of Src in extracellular vesicles (EVs) and total cell lysates (TCL) from the transduced cells were evaluated by Western blotting. 10 μg of protein from Exo or TCL were loaded. Expression levels of Src kinase, AR, Calnexin, GAPDH, and CD9 were analyzed by Western blotting. The Src protein was quantified by Image J software. The ratio of Src protein levels in EVs relative to that in TCL is shown. Data are expressed as mean±SEM. ** p<0.01.

FIG. 11 shows overexpression of Fyn kinase and loss of the palmitoylation of Fyn kinase. SYF1 (Src−/−Yes−/−Fyn−/−) cells were transduced with control vector, Fyn(WT), or Fyn(C3S/C6S) mutant by lentiviral infection. The transduced cells were incubated with/without 50 μM 17-octadecynoic acid-azide (an analog of palmitate). The cell lysates were subjected to Click chemistry through the azide-alkyne reaction, and detected with streptavidin-HRP by immunoblotting. Levels of GAPDH and Fyn were analyzed by immunoblotting.

FIG. 12 shows histology of Src transduced xenograft tumors. DU145 cells were transduced with vector control, Src(Y529F), or Src(Y529F/G2A) by lentiviral infection. The transduced cells (1×10⁴ cells/graft) were implanted sub-renally in SCID mice. After 5 weeks, the mice were sacrificed and xenograft tumors were harvested. The histology and expression levels of Src were analyzed by Haemotoxylin and Eosin (H&E) staining and immunohistochemistry (IHC), respectively. Elevated levels of Src were detected in xenograft tumors expressing Src(Y529F) and Src(Y529F/G2A).

FIG. 13 shows treatment with Filipin decreases cholesterol levels in PC3 cells. PC3 cells were treated with vehicle control or 1 μM Filipin for 24 h. The treated cells were visualized under a fluorescence microscope. The treated cells were stained with Filipin III and representative images were taken. The treatment of 1 μM Filipin inhibits the fluorescence intensity which reflects the cholesterol levels of PC3 cells.

FIGS. 14A and 14B shows loss of Src kinase myristoylation inhibits expression levels of syntenin in EVs. FIG. 4A shows DU145 cells transduced with control vector, Src(Y529F), or Src(Y529F/G2A) cells by lentiviral infection. Expression levels of syntenin, Src, calnexin, GAPDH, and CD9 in extracellular vesicles (EVs) and total cell lysate (TCL) were analyzed by immunoblotting. Ten μg of EVs or TCL were loaded according to the DC protein assay. Expression levels of syntenin and CD9 in EVs derived from DU145 expressing control vector, Src(Y529F), or Src(Y529F/G2A) were quantified using Image J software. The ratio of syntenin levels to CD9 levels in the control is set as 1. FIG. 14B shows PC3 cells transduced with shRNA-Control or shRNA-Src by lentiviral infection. The transduced cells were grown with 10% exosome-free FBS for 48 h. EVs were isolated from the conditioned medium. Expression levels of syntenin, Src, calnexin, GAPDH, and CD9 in EVs and total cell lysates were detected by immunoblotting. Syntenin and CD9 levels in EVs were quantified using Image J software. The ratio of syntenin to CD9 levels in the shRNA-control group is set as 1. Down-regulation of Src kinase decreases expression levels of syntenin in EVs. Data are expressed as mean±SEM. *: p<0.05; **: p<0.01; ***: p<0.001; ****: p<0.0001. To measure the Km and Vmax of NMT1 which catalyzed various octapeptides substrates derived from various proteins, twenty-five octapeptides were synthesized by GenScript. These peptide included Src8(G2A), a mutant octapeptide [Ala-Ser-Asn-Lys-Ser-Lys-Pro-Lys], which is not a substrate of NMT1 enzyme. Each data point has three repeats.

FIG. 15A shows that NMT1 catalyzes the incorporation of the myristoyl group into the N-terminus of the glycine in an octapeptide, such as Gly-Ser-Asn-Lys-Ser-Lys-Pro-Lys, derived from the leading sequence of Src kinase and releases CoA. The amount of the released CoA were reacted with 7-diethylamino-3-(4′-maleimidylphenyl)-4-methylcoumarin. The assay was performed in 96-well black microplates. The produced fluorescence intensity was measured by Flex Station 3, and detected by microplate reader (excitation at 390 nm; emission at 479 nm). FIG. 15B shows that docking analysis of octapeptide of derived from Src kinase with the peptide binding site of the full length NMT1 protein. The docking analysis of NMT1 with the first amino acid, and a leading peptide containing the first 2, 3, 4, 5, 6, 7, 8, 9, 10 amino acids from c-Src, indicates that a peptide with 7-8 amino acids has favorable docking with NMT1 enzyme (lower score). FIG. 15C shows that Src8(WT), but not Src8(G2A), a mutant octapeptide [Ala-Ser-Asn-Lys-Ser-Lys-Pro-Lys] was a substrate of NMT1 enzyme (Each data point had three repeats).

FIGS. 16A to 16F show myristoylation of Cas9 promotes its encapsulation into EVs, and maintains genome editing function. FIG. 16A shows the diagram of bicistron lentiviral vectors expressing Cas9/sgRNA-scramble, Cas9/sgRNA-GFP, mCas9/sgRNA-GFP, and mCas9(G2A)/sgRNA-GFP. The octapeptide DNA sequence derived from the N-terminus of Src kinase was fused with Cas9 gene, designated as mCas9. A mutation of Gly to Ala at site 2 of mCas9, designated as mCas9(G2A), were also created. The mCas9(G2A) leads to loss of myristoylation of the mCas9 protein. FIG. 16B shows that 293T-GFP cells were transduced with Cas9/sgRNA-scrambled (a negative control), Cas9/sgRNA-GFP (a positive control), mCas9/sgRNA-GFP, and mCas9(G2A)/sgRNA-GFP by lipofectamine 3000. After 5 days, the transduced cells were analyzed in the green channel by FACS analysis. The GFP negative cells were sorted out, and re-grown in DMEM medium. Images were taken of the above treatment groups. The data represent three experiments. FIG. 16C shows that the isolated GFP negative cells were cultured in the medium with 60 uM of myristic acid-azide (analog of myristic acid). The expression of Cas9 (Western Blot, anti-Flag) and myristoylated Cas9 (Click chemistry, then detected by streptavidin-HDP) were analyzed. FIG. 16A shows that T7 endonuclease analysis. The flank of PAM site of GFP gene was PCR amplified from GFP negative cells. The PCR products were digested with T7 endonuclease, and resulted in 256 bp and 170 bp fragments as expected. FIG. 16E shows that 293T-GFP cells expressing Cas9/sgRNA-scrambled (a negative control), Cas9/sgRNA-GFP (a positive control), mCas9/sgRNA-GFP, and mCas9(G2A)/sgRNA-GFP. The GFP negative cells were sorted out by FACS. EVs from the GFP negative cells were isolated using sequential ultra-centrifugation. The cell lysates (the first 4 lanes) and EVs lysates (the last 4 lanes) were analyzed for expression levels of Cas9, calnexin, CD9, GAPDH, and GFP by Western Blot. FIG. 16F shows that Total RNA was also isolated from EVs. sgRNA were PCR amplified and Sanger sequenced. The sgRNA sequence of targeting GFP gene were confirmed.

FIGS. 17A to 17E show that myristoylation promotes encapsulation of Cas9 protein into EVs. FIG. 17A shows schematic of experimental process to produce EVs from EVs-producing cells expressing mCas9/sgRNA-luciferase. 3T3 stably expressing luciferase (3T3-luc) cell line was created by transduction of luciferase gene by lentiviral infection. 3T3-luc cells were transduced Cas9, mCas9, or mCas9(G2A)/gRNA-luc by lentiviral infection. Single cell clone was selected and expanded according to expression levels of Cas9 and reduction of luciferase activity. EVs were isolated from conditioned medium from EVs-producing cells expressing Cas9, mCas9, or mCas9(G2A)/gRNA-luc. FIG. 17B shows that luciferase activity was measured in the isolated EVs-producing cells expressing Cas9, mCas9, or mCas9(G2A)/gRNA-luc. Luciferase activity is reported as relative light units normalized to the protein concentration of cell lysates. FIG. 17C shows that fusion of octapeptide facilitated Cas9 myristoylation in EVs-producing cells expressing mCas9/gRNA-luc, but not those expressing Cas9 or mCas9(G2A)/gRNA-luc. EVs-producing cells were cultured with 60 μM myristic acid-azide for 24 hrs. Expression levels of Cas9, GAPDH, and myristoylated Cas9 were detected by immunoblotting. Of note, myristoylated Cas9 was detected using antibody targeting myristoylated octapeptide. FIG. 17D shows that myristoylation of Cas9 maintained its genome editing function. Genomic DNA were isolated from EVs-producing cells. The DNA of the flanking region of the genomic editing site was PCR amplified. PCR products 357 bp were obtained using the above genome DNA and Luciferase-T7 primers, and digested by T7 Endonuclease I, which led to two cleaved bands with 208 bp and 149 bp. FIG. 17D shows that Cas9 protein was encapsulated in EVs-producing cells expressing mCas9/sgRNA-luc. EVs were isolated from EVs-producing cells expressing Cas9, mCas9, or mCas9(G2A)/gRNA-luc. Expression levels of CD9, luciferase, GAPDH, and CD81 were measured in EVs-producing cells and EVs lysates by immunoblotting.

FIG. 18A shows verification of integration of Cas9/sgRNA in EVs-producing cells expressing Cas9/sgRNA. 3T3 cells expressing luciferase were transduced with Cas9/sgRNA-Luc, mCas9/sgRNA-Luc and mCas9(G2A)/sgRNA-Luc by lentiviral infection. To detect the integration of Cas9/sgRNA in the genomic levels, genomic DNA were isolated and used for the PCR template. Additionally, the primers (U6-Cas9) covering the U6 promoter and Cas9 gene were used for PCR amplification. The integration of Cas9/sgRNA were verified in the EVs-producing cells expressing Cas9/sgRNA-Luc, mCas9/sgRNA-Luc and mCas9(G2A)/sgRNA-Luc, but not the control cells. FIG. 18B shows verification of antibody detecting myristoylated epitope. An antibody was developed using the antigen of myristoylated octapeptide, myristoyl-GSNKSKPKC. To verify the specificity of the antibody, SYF1(Src^{-/-}Yes^{-/-}Fyn^{-/-}) cells were transduced with Src(WT) or Src(G2A) by lentiviral infection. Cell lysates from SYF1 cells or the above transduced cells were subjected to immunoblotting. Expression levels of Src, GAPDH, and myristoylated Src were analyzed by immunoblotting. The antibody targeting myristoyl-octapeptide derived from the leading sequence of Src kinase specifically detected Src(WT), but not Src(G2A), a mutant with loss of myristoylation site.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Cas9 Fusion Protein

Disclosed herein is a fusion protein for gene editing, comprising a Cas9 domain that is configured to be encapsulated into EVs and to localize to the nucleus of recipient cells. The fusion should possess the following criteria: 1) it should be encapsulated into EVs; and 2) it should be taken into the recipient cells, and be localized into the nucleus for genome editing. The fusion protein can therefore contain a myristoylation domain and possess a positive charge, which allows encapsulation of the protein in EVs. As disclosed herein, palmitoylation of the peptide can significantly inhibit encapsulation and/or nucleus localization. Therefore, in some embodiments, the disclosed fusion protein contains a myristoylation domain that contains a myristoylation motif but does not contain a palmitoylation motif. Therefore, disclosed herein is a fusion protein, comprising a myristoylation domain, a Cas9 domain, and a nuclear localization signal (NLS), wherein the polypeptide is configured to be myristoylated during protein translation. In some embodiments, the fusion protein comprises a myristoylation domain that possesses a myristoylation motif and a positive charge, but does not contain a palmitoylation motif.

In some embodiments, the one or more domains of the fusion proteins are separated by a polypeptide linker.

Myristoylation Domain

Myristoylation is a lipidation modification where a myristoyl group, derived from myristic acid, is covalently attached by an amide bond to the alpha-amino group of an N-terminal glycine residue. Briefly, proteins that will become myristoylated begin with a consensus sequence Met-Gly-X-X-X-Ser/Thr (SEQ ID NO:3). The start Met is cotranslationally, proteolytically removed and the myristate is added to the exposed N-terminal glycine via a stable amide bond.

As used herein, “palmitoylation” refers the covalent attachment of fatty acids, such as palmitic acid, to cysteine. Therefore, in some embodiments, the myristoylation domain of the disclosed fusion protein does not comprises a cysteine residue.

Therefore, in some cases, the myristoylation domain comprises the amino acid sequence G-X-X-X-S/T (SEQ ID NO:1), wherein X is any amino acid other than Cys. In some embodiments, the myristoylation domain comprises the amino acid sequence GSNKS (SEQ ID NO:340). In some cases, the myristoylation domain comprises 5 to 10 amino acids, including 5, 6, 7, 8, 9, or 10 amino acids. Therefore, in some cases, the myristoylation domain comprises the amino acid sequence G-X₁-X₁-X₁-S/T-X₂-X₂-X₂-X₂-X₂ (SEQ ID NO:2), wherein X₁ is any amino acid other than Cys, and wherein X₂ is a basic amino acid, any amino acid, or nothing. For example, in some embodiments, the myristoylation domain comprises or consists of the amino acid sequence GSNKSKPKDA (SEQ ID NO:341). In some cases, the myristoylation domain is encoded by the nucleic acid sequence

(SEQ ID NO: 344)
GGCAGCAACAAGAGCAAGCCCAAG.

Cas9 Domain

The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNA. However, single guide RNAs (“sgRNA”, or simply “gNRA”) can be engineered so as to incorporate aspects of both the crRNA and tracrRNA into a single RNA species. See e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain.

In some embodiments, the Cas9 domain comprises wild type Cas9 from Streptococcus pyogenes (NCBI Reference Sequence: NC_017053.1. Therefore, in some embodiments, the Cas9 domain comprise the amino acid sequence:

(SEQ ID NO: 4)
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQ1ANLAGSPAIKKG1LQTVKIVDELVKV
MGHKPENIVIEMARENQTTQKGQKNSRERMKRIEEGIKELGSQ1LKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD.

In some embodiments, the Cas9 domain comprises the amino acid sequence:

(SEQ ID NO: 5)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAVVMTRKSEETITPWNFEEVVDKGASAQSFIERMTNFD
KNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEH
PVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKL
IREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD.

In some embodiments, the Cas9 domain comprises wild type Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1).

In some embodiments, the Cas9 domain is nuclease-inactive. Point mutations can be introduced into Cas9 to abolish nuclease activity, resulting in a dead Cas9 (dCas9) that still retains its ability to bind DNA in a sgRNA-programmed manner. In principle, when fused to another protein or domain, dCas9 can target that protein to virtually any DNA sequence simply by co-expression with an appropriate sgRNA. Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821(2012); Qi et al., Cell. 28; 152(5):1173-83 (2013).

For example, in some embodiments, the Cas9 domain comprises the amino acid sequence:

(dCas9 with D10A and H840A, SEQ ID NO: 6)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAVVMTRKSEETITPWNFEEVVDKGASAQSFIERMTNFD
KNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEH
PVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKL
IREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEI1EQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD.

In some embodiments, the Cas9 domain is encoded by the nucleic acid sequence:

(SEQ ID NO: 345)
ATGGGCAGCAACAAGAGCAAGCCCAAGGATAAGAAATACTCAATAGGACT
GGATATTGGCACAAATAGCGTCGGATGGGCTGTGATCACTGATGAATATA
AGGTTCCTTCTAAAAAGTTCAAGGTTCTGGGAAATACAGACCGCCACAGT
ATCAAAAAAAATCTTATAGGGGCTCTTCTGTTTGACAGTGGAGAGACAGC
CGAAGCTACTAGACTCAAACGGACAGCTAGGAGAAGGTATACAAGACGGA
AGAATAGGATTTGTTATCTCCAGGAGATTTTTTCAAATGAGATGGCCAAA
GTGGATGATAGTTTCTTTCATAGACTTGAAGAGTCTTTTTTGGTGGAAGA
AGACAAGAAGCATGAAAGACATCCTATTTTTGGAAATATAGTGGATGAAG
TTGCTTATCACGAGAAATATCCAACTATCTATCATCTGAGAAAAAAATTG
GTGGATTCTACTGATAAAGCCGATTTGCGCCTGATCTATTTGGCCCTGGC
CCACATGATTAAGTTTAGAGGTCATTTTTTGATTGAGGGCGATCTGAATC
CTGATAATAGTGATGTGGACAAACTGTTTATCCAGTTGGTGCAAACCTAC
AATCAACTGTTTGAAGAAAACCCTATTAACGCAAGTGGAGTGGATGCTAA
AGCCATTCTTTCTGCAAGATTGAGTAAATCAAGAAGACTGGAAAATCTCA
TTGCTCAGCTCCCCGGTGAGAAGAAAAATGGCCTGTTTGGGAATCTCATT
GCTTTGTCATTGGGTTTGACCCCTAATTTTAAATCAAATTTTGATTTGGC
AGAAGATGCTAAACTCCAGCTTTCAAAAGATACTTACGATGATGATCTGG
ATAATCTGTTGGCTCAAATTGGAGATCAATATGCTGATTTGTTTTTGGCA
GCTAAGAATCTGTCAGATGCTATTCTGCTTTCAGACATCCTGAGAGTGAA
TACTGAAATAACTAAGGCTCCCCTGTCAGCTTCAATGATTAAACGCTACG
ATGAACATCATCAAGACTTGACTCTTCTGAAAGCCCTGGTTAGACAACAA
CTTCCAGAAAAGTATAAAGAAATCTTTTTTGATCAATCAAAAAACGGATA
TGCAGGTTATATTGATGGCGGCGCAAGCCAAGAAGAATTTTATAAATTTA
TCAAACCAATTCTGGAAAAAATGGATGGTACTGAGGAACTGTTGGTGAAA
CTGAATAGAGAAGATTTGCTGCGCAAGCAACGGACCTTTGACAACGGCTC
TATTCCCCATCAAATTCACTTGGGTGAGCTGCATGCTATTTTGAGAAGAC
AAGAAGACTTTTATCCATTTCTGAAAGACAATAGAGAGAAGATTGAAAAA
ATCTTGACTTTTAGGATTCCTTATTATGTTGGTCCATTGGCCAGAGGCAA
TAGTAGGTTTGCATGGATGACTCGGAAGTCTGAAGAAACAATTACCCCAT
GGAATTTTGAAGAAGTTGTCGATAAAGGTGCTTCAGCTCAATCATTTATT
GAACGCATGACAAACTTTGATAAAAATCTTCCAAATGAAAAAGTGCTGCC
AAAACATAGTTTGCTTTATGAGTATTTTACCGTTTATAACGAATTGACAA
AGGTCAAATATGTTACTGAAGGAATGAGAAAACCAGCATTTCTTTCAGGT
GAACAGAAGAAAGCCATTGTTGATCTGCTCTTCAAAACAAATAGGAAAGT
GACCGTTAAGCAACTGAAAGAAGATTATTTCAAAAAAATAGAATGTTTTG
ATAGTGTTGAAATTTCAGGAGTTGAAGATAGATTTAATGCTTCACTGGGT
ACATACCATGATTTGCTGAAAATTATTAAAGATAAAGATTTTTTGGATAA
TGAAGAAAATGAAGACATCCTGGAGGATATTGTTCTGACATTGACCCTGT
TTGAAGATAGGGAGATGATTGAGGAAAGACTTAAAACATACGCTCACCTC
TTTGATGATAAGGTGATGAAACAGCTTAAAAGACGCAGATATACTGGTTG
GGGAAGGTTGTCCAGAAAATTGATTAATGGTATTAGGGATAAGCAATCTG
GCAAAACAATACTGGATTTTTTGAAATCAGATGGTTTTGCCAATCGCAAT
TTTATGCAGCTCATCCATGATGATAGTTTGACATTTAAAGAAGACATCCA
AAAAGCACAAGTGTCTGGACAAGGCGATAGTCTGCATGAACATATTGCAA
ATCTGGCTGGTAGCCCTGCTATTAAAAAAGGTATTCTCCAGACTGTGAAA
GTTGTTGATGAATTGGTCAAAGTGATGGGGCGGCATAAGCCAGAAAATAT
CGTTATTGAAATGGCAAGAGAAAATCAGACAACTCAAAAGGGCCAGAAAA
ATTCCAGAGAGAGGATGAAAAGAATCGAAGAAGGTATCAAAGAACTGGGA
AGTCAGATTCTTAAAGAGCATCCTGTTGAAAATACTCAATTGCAAAATGA
AAAGCTCTATCTCTATTATCTCCAAAATGGAAGAGATATGTATGTGGACC
AAGAACTGGATATTAATAGGCTGAGTGATTATGATGTCGATCACATTGTT
CCACAAAGTTTCCTTAAAGACGATTCAATAGACAATAAGGTCCTGACCAG
GTCTGATAAAAATAGAGGTAAATCCGATAACGTTCCAAGTGAAGAAGTGG
TCAAAAAGATGAAAAACTATTGGAGACAACTTCTGAACGCCAAGCTGATC
ACTCAAAGGAAGTTTGATAATCTGACCAAAGCTGAAAGAGGAGGTTTGAG
TGAACTTGATAAAGCTGGTTTTATCAAACGCCAATTGGTTGAAACTCGCC
AAATCACTAAGCATGTGGCACAAATTTTGGATAGTCGCATGAATACTAAA
TACGATGAAAATGATAAACTTATTAGAGAGGTTAAAGTGATTACCCTGAA
ATCTAAACTGGTTTCTGACTTCAGAAAAGATTTCCAATTCTATAAAGTGA
GAGAGATTAACAATTACCATCATGCCCATGATGCCTATCTGAATGCCGTC
GTTGGAACTGCTTTGATTAAGAAATATCCAAAACTTGAAAGCGAGTTTGT
CTATGGTGATTATAAAGTTTATGATGTTAGGAAAATGATTGCTAAGTCTG
AGCAAGAAATAGGCAAAGCAACCGCAAAGTATTTCTTTTACTCTAATATC
ATGAACTTCTTCAAAACAGAAATTACACTTGCAAATGGAGAGATTCGCAA
ACGCCCTCTGATCGAAACTAATGGGGAAACTGGAGAAATTGTCTGGGATA
AAGGGAGAGATTTTGCCACAGTGCGCAAAGTGTTGTCCATGCCCCAAGTC
AATATCGTCAAGAAAACAGAAGTGCAGACAGGCGGATTCTCTAAGGAGTC
AATTCTGCCAAAAAGAAATTCCGACAAGCTGATTGCTAGGAAAAAAGACT
GGGACCCAAAAAAATATGGTGGTTTTGATAGTCCAACCGTGGCTTATTCA
GTCCTGGTGGTTGCTAAGGTGGAAAAAGGGAAATCCAAGAAGCTGAAATC
CGTTAAAGAGCTGCTGGGGATCACAATTATGGAAAGAAGTTCCTTTGAAA
AAAATCCCATTGACTTTCTGGAAGCTAAAGGATATAAGGAAGTTAAAAAA
GACCTGATCATTAAACTGCCTAAATATAGTCTTTTTGAGCTGGAAAACGG
TAGGAAACGGATGCTGGCTAGTGCCGGAGAACTGCAAAAAGGAAATGAGC
TGGCTCTGCCAAGCAAATATGTGAATTTTCTGTATCTGGCTAGTCATTAT
GAAAAGTTGAAGGGTAGTCCAGAAGATAACGAACAAAAACAATTGTTTGT
GGAGCAGCATAAGCATTATCTGGATGAGATTATTGAGCAAATCAGTGAAT
TTTCTAAGAGAGTTATTCTGGCAGATGCCAATCTGGATAAAGTTCTTAGT
GCATATAACAAACATAGAGACAAACCAATAAGAGAACAAGCAGAAAATAT
CATTCATCTGTTTACCTTGACCAATCTTGGAGCACCCGCTGCTTTTAAAT
ACTTTGATACAACAATTGATAGGAAAAGATATACCTCTACAAAAGAAGTT
CTGGATGCCACTCTTATCCATCAATCCATCACTGGTCTTTATGAAACACG
CATTGATTTGAGTCAGCTGGGAGGTGAC.

In some embodiments, the Cas9 domain is a Cas9 variant. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of Cas9.

Nuclear Localization Signal (NLS)

In some embodiments, the NLS sequence comprises, in part or in whole, the amino acid sequence of one or dual SV40 NLS sequence (PKKKRKV, SEQ ID NO:342). In some embodiments, the NLS sequence comprises, in part or in whole, the amino acid sequence nucleoplasmin (AVKRPAATKKAGQAKKKKLD, SEQ ID NO: 343), EGL-13 (MSRRRKANPTKLSENAKKLAKEVEN, SEQ ID NO: 344), c-Myc (PAAKRVKLD, SEQ ID NO: 345), orTUS-protein (KLKIKRPVK, SEQ ID NO: 346). In some embodiments, the NLS sequence is encoded by the nucleic acid sequence CCCAAGAAAAAACGCAAGGTG (SEQ ID NO:347), CCTAAGAAAAAGCGGAAAGTG (SEQ ID NO:348), or a combination thereof.

Additional features may be present, for example, one or more linker sequences between the NLS and the rest of the fusion protein and/or between the nucleic acid-editing enzyme or domain and the Cas9. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. For example, in some embodiments, a myc tag is encoded by the nucleic acid sequence GAGCAGAAACTCATCTCAGAAGAGGATCTG (SEQ ID NO:349). For example, in some embodiments, a FLAG tag is encoded by the nucleic acid sequence

(SEQ ID NO: 350)
GATTACAAGGATGACGACGATAAG.

In some embodiments, the polynucleotide encoding the disclosed fusion protein comprises the nucleic acid sequence:

(SEQ ID NO: 351)
GTCGACGGATCGGGAGATCTCCCGATCCCCTATGGTGCACTCTCAGTACAATCTGCTC
TGATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATGAA
GAATCTGCTTAGGGTTAGGCGTTTTGCGCTGCTTCGCGATGTACGGGCCAGATATACG
CGTTGACATTGATTATTGACTAGTTATTAATAGTAATCAATTACGGGGTCATTAGTTCATA
GCCCATATATGGAGTTCCGCGTTACATAACTTACGGTAAATGGCCCGCCTGGCTGACC
GCCCAACGACCCCCGCCCATTGACGTCAATAATGACGTATGTTCCCATAGTAACGCCAA
TAGGGACTTTCCATTGACGTCAATGGGTGGACTATTTACGGTAAACTGCCCACTTGGCA
GTACATCAAGTGTATCATATGCCAAGTACGCCCCCTATTGACGTCAATGACGGTAAATG
GCCCGCCTGGCATTATGCCCAGTACATGACCTTACGGGACTTTCCTACTTGGCAGTACA
TCTACGTATTAGTCATCGCTATTACCATGGTGATGCGGTTTTGGCAGTACACCAATGGG
CGTGGATAGCGGTTTGACTCACGGGGATTTCCAAGTCTCCACCCCATTGACGTCAATG
GGAGTTTGTTTTGGCACCAAAATCAACGGGACTTTCCAAAATGTCGTAACAACTCCGCC
CCATTGACGCAAATGGGCGGTAGGCGTGTACGGTGGGAGGTCTCTGTACTGGGTCTCT
CTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAGTG
GCGCCCGAACAGGGACTTGAAAGCGAAAGGGAAACCAGAGGAGCTCTCTCGACGCAG
GACTCGGCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTA
CAACCATCCCTTCAGACAGGATCAGAAGAACTTAGATCATTATATAATACAGTAGCAACC
CTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAG
ACCTGGAGGAGGAGATATGAGGGACAATTGGAGAAGTGAATTATATAAATATAAAGTAG
TAAAAATTGAACCATTAGGAGTAGCACCCACCAAGGCAAAGAGAAGAGTGGTGCAGAG
AGAAAAAAGAGCAGTGGGAATAGGAGCTTTGTTCCTTGGGTTCTTGGGAGCAGCAGGA
AGCACTATGGGCGCAGCGTCAATGACGCTGACGGTACAGGCCAGACAATTATTGTCTG
GTATAGTGCAGCAGCAGAACAATTTGCTGAGGGCTATTGAGGCGCAACAGCATCTGTT
GCAACTCACAGTCTGGGGCATCAAGCAGCTCCAGGCAAGAATCCTGGCTGTGGAAAGA
TACCTAAAGGATCAACAGCTCCTGGGGATTTGGGGTTGCTCTGGAAAACTCATTTGCAC
CACTGCTGTGCCTTGGAATGCTAGTTGGAGTAATAAATCTCTGGAACAGATTTGGAATC
ACACGACCTGGATGGAGTGGGACAGAGAAATTAACAATTACACAAGCTTAATACACTCC
TTAATTGAAGAATCGCAAAACCAGCAAGAAAAGAATGAACAAGAATTATTGGAATTAGAT
AAATGGGCAAGTTTGTGGAATTGGTTTAACATAACAAATTGGCTGTGGTATATAAAATTA
TTCATAATGATAGTAGGAGGCTTGGTAGGTTTAAGAATAGTTTTTGCTGTACTTTCTATA
GTGAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGAGA
CAGATCCATTCGATTAGTGAACGGATCGGCACTGCGTGCGCCAATTCTGCAGACAAAT
GGCAGTATTCATCCACAATTTTAAAAGAAAAGGGGGGATTGGGGGGTACAGTGCAGGG
GAAAGAATAGTAGAAATAATAGCAACAGACATACAAACTAAAGAATTACAAAAACAAATT
ACAAAAATTCAAAATTTTCGGGTTTATTACAGGGACAGCAGAGATCCAGTTTGGTTAATC
CGCTAGCTCTAGAGGATCTGAATTCCCCAGTGGAAAGACGCGCAGGCAAAACGCACCA
CGTGACGGAGCGTGACCGCGCGCCGAGCGCGCGCCAAGGTCGGGCAGGAAGAGGGC
CTATTTCCCATGATTCCTTCATATTTGCATATACGATACAAGGCTGTTAGAGAGATAATT
AGAATTAATTTGACTGTAAACACAAAGATATTAGTACAAAATACGTGACGTAGAAAGTAA
TAATTTCTTGGGTAGTTTGCAGTTTTAAAATTATGTTTTAAAATGGACTATCATATGCTTA
CCGTAACTTGAAAGTATTTCGATTTCTTGGGTTTATATATCTTGTGGAAAGGACGCGGG
ATCCACTGGACCAGGCAGCAGCGTCAGAAGACTTTTTTGGAACGTCTCGTTTTAGAGCT
AGAAATAGCAAGTTAAAATAAGGCTAGTCCGTTATCAACTTGAAAAAGTGGCACCGAGT
CGGTGCTTTTTTTGGTGTACATTTATATTGGCTCATGTCCAATATGACCGCCATGTTGAC
ATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATC
AAGTGTATCATATGCCAAGTCCGCCCCCTATTGACGTCAATGACGGTAAATGGCCCGC
CTGGCATTATGCCCAGTACATGACCTTACGGGACTTTCCTACTTGGCAGTACATCTACG
TATTAGTCATCGCTATTACCATGGTGATGCGGTTTTGGCAGTACACCAATGGGCGTGGA
TAGCGGTTTGACTCACGGGGATTTCCAAGTCTCCACCCCATTGACGTCAATGGGAGTTT
GTTTTGGCACCAAAATCAACGGGACTTTCCAAAATGTCGTAATAACCCCGCCCCGTTGA
CGCAAATGGGCGGTAGGCGTGTACGGTGGGAGGTCTATATAAGCAGAGCTCGTTTAGT
GAACCGTCAGAATTTTGTAATACGACTCACTATAGGGCGGCCGGGAATTCGTCGACTG
GAACCGGTACCGAGGAGATCTGCCGCCGCGATCGCCATGGGCAGCAACAAGAGCAAG
CCCAAGGATAAGAAATACTCAATAGGACTGGATATTGGCACAAATAGCGTCGGATGGG
CTGTGATCACTGATGAATATAAGGTTCCTTCTAAAAAGTTCAAGGTTCTGGGAAATACAG
ACCGCCACAGTATCAAAAAAAATCTTATAGGGGCTCTTCTGTTTGACAGTGGAGAGACA
GCCGAAGCTACTAGACTCAAACGGACAGCTAGGAGAAGGTATACAAGACGGAAGAATA
GGATTTGTTATCTCCAGGAGATTTTTTCAAATGAGATGGCCAAAGTGGATGATAGTTTCT
TTCATAGACTTGAAGAGTCTTTTTTGGTGGAAGAAGACAAGAAGCATGAAAGACATCCT
ATTTTTGGAAATATAGTGGATGAAGTTGCTTATCACGAGAAATATCCAACTATCTATCAT
CTGAGAAAAAAATTGGTGGATTCTACTGATAAAGCCGATTTGCGCCTGATCTATTTGGC
CCTGGCCCACATGATTAAGTTTAGAGGTCATTTTTTGATTGAGGGCGATCTGAATCCTG
ATAATAGTGATGTGGACAAACTGTTTATCCAGTTGGTGCAAACCTACAATCAACTGTTTG
AAGAAAACCCTATTAACGCAAGTGGAGTGGATGCTAAAGCCATTCTTTCTGCAAGATTG
AGTAAATCAAGAAGACTGGAAAATCTCATTGCTCAGCTCCCCGGTGAGAAGAAAAATGG
CCTGTTTGGGAATCTCATTGCTTTGTCATTGGGTTTGACCCCTAATTTTAAATCAAATTTT
GATTTGGCAGAAGATGCTAAACTCCAGCTTTCAAAAGATACTTACGATGATGATCTGGA
TAATCTGTTGGCTCAAATTGGAGATCAATATGCTGATTTGTTTTTGGCAGCTAAGAATCT
GTCAGATGCTATTCTGCTTTCAGACATCCTGAGAGTGAATACTGAAATAACTAAGGCTC
CCCTGTCAGCTTCAATGATTAAACGCTACGATGAACATCATCAAGACTTGACTCTTCTGA
AAGCCCTGGTTAGACAACAACTTCCAGAAAAGTATAAAGAAATCTTTTTTGATCAATCAA
AAAACGGATATGCAGGTTATATTGATGGCGGCGCAAGCCAAGAAGAATTTTATAAATTT
ATCAAACCAATTCTGGAAAAAATGGATGGTACTGAGGAACTGTTGGTGAAACTGAATAG
AGAAGATTTGCTGCGCAAGCAACGGACCTTTGACAACGGCTCTATTCCCCATCAAATTC
ACTTGGGTGAGCTGCATGCTATTTTGAGAAGACAAGAAGACTTTTATCCATTTCTGAAAG
ACAATAGAGAGAAGATTGAAAAAATCTTGACTTTTAGGATTCCTTATTATGTTGGTCCAT
TGGCCAGAGGCAATAGTAGGTTTGCATGGATGACTCGGAAGTCTGAAGAAACAATTAC
CCCATGGAATTTTGAAGAAGTTGTCGATAAAGGTGCTTCAGCTCAATCATTTATTGAACG
CATGACAAACTTTGATAAAAATCTTCCAAATGAAAAAGTGCTGCCAAAACATAGTTTGCT
TTATGAGTATTTTACCGTTTATAACGAATTGACAAAGGTCAAATATGTTACTGAAGGAAT
GAGAAAACCAGCATTTCTTTCAGGTGAACAGAAGAAAGCCATTGTTGATCTGCTCTTCA
AAACAAATAGGAAAGTGACCGTTAAGCAACTGAAAGAAGATTATTTCAAAAAAATAGAAT
GTTTTGATAGTGTTGAAATTTCAGGAGTTGAAGATAGATTTAATGCTTCACTGGGTACAT
ACCATGATTTGCTGAAAATTATTAAAGATAAAGATTTTTTGGATAATGAAGAAAATGAAGA
CATCCTGGAGGATATTGTTCTGACATTGACCCTGTTTGAAGATAGGGAGATGATTGAGG
AAAGACTTAAAACATACGCTCACCTCTTTGATGATAAGGTGATGAAACAGCTTAAAAGAC
GCAGATATACTGGTTGGGGAAGGTTGTCCAGAAAATTGATTAATGGTATTAGGGATAAG
CAATCTGGCAAAACAATACTGGATTTTTTGAAATCAGATGGTTTTGCCAATCGCAATTTT
ATGCAGCTCATCCATGATGATAGTTTGACATTTAAAGAAGACATCCAAAAAGCACAAGT
GTCTGGACAAGGCGATAGTCTGCATGAACATATTGCAAATCTGGCTGGTAGCCCTGCTA
TTAAAAAAGGTATTCTCCAGACTGTGAAAGTTGTTGATGAATTGGTCAAAGTGATGGGG
CGGCATAAGCCAGAAAATATCGTTATTGAAATGGCAAGAGAAAATCAGACAACTCAAAA
GGGCCAGAAAAATTCCAGAGAGAGGATGAAAAGAATCGAAGAAGGTATCAAAGAACTG
GGAAGTCAGATTCTTAAAGAGCATCCTGTTGAAAATACTCAATTGCAAAATGAAAAGCTC
TATCTCTATTATCTCCAAAATGGAAGAGATATGTATGTGGACCAAGAACTGGATATTAAT
AGGCTGAGTGATTATGATGTCGATCACATTGTTCCACAAAGTTTCCTTAAAGACGATTCA
ATAGACAATAAGGTCCTGACCAGGTCTGATAAAAATAGAGGTAAATCCGATAACGTTCC
AAGTGAAGAAGTGGTCAAAAAGATGAAAAACTATTGGAGACAACTTCTGAACGCCAAGC
TGATCACTCAAAGGAAGTTTGATAATCTGACCAAAGCTGAAAGAGGAGGTTTGAGTGAA
CTTGATAAAGCTGGTTTTATCAAACGCCAATTGGTTGAAACTCGCCAAATCACTAAGCAT
GTGGCACAAATTTTGGATAGTCGCATGAATACTAAATACGATGAAAATGATAAACTTATT
AGAGAGGTTAAAGTGATTACCCTGAAATCTAAACTGGTTTCTGACTTCAGAAAAGATTTC
CAATTCTATAAAGTGAGAGAGATTAACAATTACCATCATGCCCATGATGCCTATCTGAAT
GCCGTCGTTGGAACTGCTTTGATTAAGAAATATCCAAAACTTGAAAGCGAGTTTGTCTAT
GGTGATTATAAAGTTTATGATGTTAGGAAAATGATTGCTAAGTCTGAGCAAGAAATAGGC
AAAGCAACCGCAAAGTATTTCTTTTACTCTAATATCATGAACTTCTTCAAAACAGAAATTA
CACTTGCAAATGGAGAGATTCGCAAACGCCCTCTGATCGAAACTAATGGGGAAACTGG
AGAAATTGTCTGGGATAAAGGGAGAGATTTTGCCACAGTGCGCAAAGTGTTGTCCATGC
CCCAAGTCAATATCGTCAAGAAAACAGAAGTGCAGACAGGCGGATTCTCTAAGGAGTC
AATTCTGCCAAAAAGAAATTCCGACAAGCTGATTGCTAGGAAAAAAGACTGGGACCCAA
AAAAATATGGTGGTTTTGATAGTCCAACCGTGGCTTATTCAGTCCTGGTGGTTGCTAAG
GTGGAAAAAGGGAAATCCAAGAAGCTGAAATCCGTTAAAGAGCTGCTGGGGATCACAA
TTATGGAAAGAAGTTCCTTTGAAAAAAATCCCATTGACTTTCTGGAAGCTAAAGGATATA
AGGAAGTTAAAAAAGACCTGATCATTAAACTGCCTAAATATAGTCTTTTTGAGCTGGAAA
ACGGTAGGAAACGGATGCTGGCTAGTGCCGGAGAACTGCAAAAAGGAAATGAGCTGG
CTCTGCCAAGCAAATATGTGAATTTTCTGTATCTGGCTAGTCATTATGAAAAGTTGAAGG
GTAGTCCAGAAGATAACGAACAAAAACAATTGTTTGTGGAGCAGCATAAGCATTATCTG
GATGAGATTATTGAGCAAATCAGTGAATTTTCTAAGAGAGTTATTCTGGCAGATGCCAAT
CTGGATAAAGTTCTTAGTGCATATAACAAACATAGAGACAAACCAATAAGAGAACAAGC
AGAAAATATCATTCATCTGTTTACCTTGACCAATCTTGGAGCACCCGCTGCTTTTAAATA
CTTTGATACAACAATTGATAGGAAAAGATATACCTCTACAAAAGAAGTTCTGGATGCCAC
TCTTATCCATCAATCCATCACTGGTCTTTATGAAACACGCATTGATTTGAGTCAGCTGGG
AGGTGACCCCAAGAAAAAACGCAAGGTGGAAGATCCTAAGAAAAAGCGGAAAGTGGAC
ACGCGTACGCGGCCGCTCGAGCAGAAACTCATCTCAGAAGAGGATCTGGCAGCAAATG
ATATCCTGGATTACAAGGATGACGACGATAAGGTTTAACTTAATTAATTCGATATCAAGC
TTATCGATAATCAACCTCTGGATTACAAAATTTGTGAAAGATTGACTGGTATTCTTAACTA
TGTTGCTCCTTTTACGCTATGTGGATACGCTGCTTTAATGCCTTTGTATCATGCTATTGC
TTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTCGCTT
TCCCCCTCCCTATTGCCACGGCGGAACTCATCGCCCGCCTGCCTTGCCCGCTGCTGGA
CAGGGGCTCGGCTGTTGGGCACTGACAATTCCGTGGTGTTGTCGGGGAAATCATCGTC
CTTTCCTTGGCTGCTCGCCTGTGTTGCCACCTGGATTCTGCGCGGGACGTCCTTCTGC
TACGTCCTTCGGCCCTCAATCCAAGCGGACCTTCCTTCCCGCGGCCTGCTGCCGGCTC
TGCGGGCCTCTTCCGCGTCTTTCGCCTTCGCCCTCAGACGAGTCGGATCTCCCTTTGG
GCGCTCCCCGCATCGATGTCGACCTCGAGACCGGCCGAACTCGAAGACCTAGAAAAAA
CATTGGAGCAATCACAAGTAGCAATACAGCAGCTACCAATGCTGATTGTGCCTGGCTAG
AAGCACAAGAGGAGGAGGAGGTGGGTTTTCCAGTCACACCTCAGGTACCTTTAAGACC
AATGACTTACAAGGCAGCTGTAGATCTTAGCCACTTTTTAAAAGAAAAGGGGGGACTGG
AAGGGCTAATTCACTCCCAACGAAGACAAGATATCCTTGATCTGTGGATCTACCACACA
CAAGGCTACTTCCCTGATTGGCAGAACTACACACCAGGGCCAGGGATCAGATATCCAC
TGACCTTTGGATGGTGCTACAAGCTAGTACCAGTTGAGCAAGAGAAGGTAGAAGAAGC
CAATGAAGGAGAGAACACCCGCTTGTTACACCCTGTGAGCCTGCATGGGATGGATGAC
CCGGAGAGAGAAGTATTAGAGTGGAGGTTTGACAGCCGCCTAGCATTTCATCACATGG
CCCGAGAGCTGCATCCGGACTGTACTGGGTCTCTCTGGTTAGACCAGATCTGAGCCTG
GGAGCTCTCTGGCTAACTAGGGAACCCACTGCTTAAGCCTCAATAAAGCTTGCCTTGAG
TGCTTCAAGTAGTGTGTGCCCGTCTGTTGTGTGACTCTGGTAACTAGAGATCCCTCAGA
CCCTTTTAGTCAGTGTGGAAAATCTCTAGCAGGGCCCGTTTAAACCCGCTGATCAGCCT
CGACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGCTTC
TGAGGCGGAAAGAACCAGCTGGGGCTCTAGGGGGTATCCCCACGCGCCCTGTAGCGG
CGCATTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCCGGCT
TTCCCCGTCAAGCTCTAAATCGGGGCATCCCTTTAGGGTTCCGATTTAGTGCTTTACGG
CACCTCGACCCCAAAAAACTTGATTAGGGTGATGGTTCACGTAGTGGGCCATCGCCCT
GATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATT
TTGGGGATTTCGGCCTATTGGTTAAAAAATGAGCTGATTTAACAAAAATTTAACGCGAAT
TAATTCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCA
GGCTCCCCAGCAGGCAGAAGTATGCAAAGCATGCATCTCAATTAGTCAGCAACCATAG
TCCCGCCCCTAACTCCGCCCATCCCGCCCCTAACTCCGCCCAGTTCCGCCCATTCTCC
GCCCCATGGCTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGCAAAAAGCTC
CCGGGAGCTTGTATATCCATTTTCGGATCTGATCAGCACGTGTTGACAATTAATCATCG
GCATAGTATATCGGCATAGTATAATACGACAAGGTGAGGAACTAAACCATGGCCAAGTT
GACCAGTGCCGTTCCGGTGCTCACCGCGCGCGACGTCGCCGGAGCGGTCGAGTTCTG
GACCGACCGGCTCGGGTTCTCCCGGGACTTCGTGGAGGACGACTTCGCCGGTGTGGT
CCGGGACGACGTGACCCTGTTCATCAGCGCGGTCCAGGACCAGGTGGTGCCGGACAA
CACCCTGGCCTGGGTGTGGGTGCGCGGCCTGGACGAGCTGTACGCCGAGTGGTCGG
AGGTCGTGTCCACGAACTTCCGGGACGCCTCCGGGCCGGCCATGACCGAGATCGGCG
AGCAGCCGTGGGGGCGGGAGTTCGCCCTGCGCGACCCGGCCGGCAACTGCGTGCAC
TTCGTGGCCGAGGAGCAGGACTGACACGTGCTACGAGATTTCGATTCCACCGCCGCCT
TCTATGAAAGGTTGGGCTTCGGAATCGTTTTCCGGGACGCCGGCTGGATGATCCTCCA
GCGCGGGGATCTCATGCTGGAGTTCTTCGCCCACCCCAACTTGTTTATTGCAGCTTATA
ATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGAC
CTCTAGCTAGAGCTTGGCGTAATCATGGTCATAGCTGTTTCCTGTGTGAAATTGTTATCC
GCTCACAATTCCACACAACATACGAGCCGGAAGCATAAAGTGTAAAGCCTGGGGTGCC
TAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTT
GCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCAATGCTCACGCTGTAG
GTATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGG
ACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAAG
GATCTTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGA
AAAGTGCCACCTGAC.

Extracellular Vesicles

Disclosed herein is a gene editing composition that comprises an extracellular vesicle (EV) encapsulating the Cas9 fusion protein disclosed herein and a guide RNA. Exemplary extracellular vesicles may include but are not limited to exosomes. However, the term “extracellular vesicles” should be interpreted to include all nanometer-scale lipid vesicles that are secreted by cells such as secreted vesicles formed from lysosomes.

EVs are cell-derived vesicles with a closed double-layer membrane structure. According to their size and density, EVs mainly include exosomes (30-150 nm), micro vesicles (MVs) (100-1000 nm), and apoptotic bodies or cancer related oncosomes (1-10 μm). EVs are able to carry various molecules, such as proteins, lipids and RNAs on their surface as well as within their lumen. The EV and exosomal surface proteins can mediate organ-specific homing of circulating EVs.

EVs are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells and tumor cells. EVs for use in the disclosed compositions and methods can be derived from any suitable cells, including the cells identified above. EVs have also been isolated from physiological fluids, such as plasma, urine, amniotic fluid and malignant effusions. Non-limiting examples of suitable EVs producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells.

EVs can also be obtained from any autologous patient-derived, heterologous haplotype-matched or heterologous stem cells so to reduce or avoid the generation of an immune response in a patient to whom the EVs are delivered. Any EV-producing cell can be used for this purpose.

EVs produced from cells can be collected from the culture medium by any suitable method. Typically a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters (for example, 0.22 μiη filter), gradient ultracentrifugation (for example, with sucrose gradient) or a combination of these methods.

In one embodiment, the EVs comprising the disclosed fusion protein are obtained by culturing a cell expressing the fusion protein and subsequently isolating indirectly modified EVs from the culture medium.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

EVs may be administered within a pharmaceutically-acceptable diluent, carrier, or excipient, in unit dosage form. Conventional pharmaceutical practice may be employed to provide suitable formulations or compositions to administer the compounds to patients suffering from a disease (e.g., cancer). Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

The disclosed extracellular vesicles further may comprise an agent, such as a therapeutic agent, where the extracellular vesicles deliver the agent to a target cell. Agents comprised by the extracellular vesicles may include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA as a so-called “cargo RNA.” For example, in some embodiments the fusion protein further may comprise an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “targeting protein” and a “packaging protein.” In some embodiments, the packaging protein may be referred to as extracellular vesicle-loading protein or “EV-loading protein.” (See Hung and Leonard, “A platform for actively loading cargo RNA to elucidate limiting steps in EV-mediated delivery,” J. Extracellular Vesicles, 2016, 5: 31027, published 13 May 2016, the content of which is incorporated herein by reference in its entirety.)

### Methods for DNA Editing

Disclosed herein are methods for editing DNA in a cell with a gene editing composition disclosed herein. In some embodiments, any of the methods provided herein can be performed on DNA in a cell, for example a bacterium, a yeast cell, or a mammalian cell. In some embodiments, the DNA contacted by any Cas9 protein provided herein is in a eukaryotic cell. In some embodiments, the methods can be performed on a cell or tissue in vitro or ex vivo. In some embodiments, the eukaryotic cell is in an individual, such as a patient or research animal. In some embodiments, the individual is a human.

### Polynucleotides, Vectors, Cells, Kits

Also disclosed herein are polynucleotides encoding one or more of the proteins and/or gRNAs described herein. For example, polynucleotides encoding any of the proteins described herein are provided, e.g., for recombinant expression and purification. In some embodiments, an isolated polynucleotides comprises one or more sequences encoding a gRNA, alone or in combination with a sequence encoding any of the proteins described herein.

In some embodiments, vectors encoding any of the proteins described herein are provided, e.g., for recombinant expression and purification of Cas9 proteins, and/or fusions comprising Cas9 fusion proteins. In some embodiments, the vector comprises or is engineered to include an isolated polynucleotide, e.g., those described herein. In some embodiments, the vector comprises one or more sequences encoding a Cas9 fusion protein (as described herein), a gRNA, or combinations thereof, as described herein. Typically, the vector comprises a sequence encoding the fusion protein operably linked to a promoter, such that the fusion protein is expressed in a host cell.

In some embodiments, cells are provided, e.g., for recombinant expression and encapsulation of the disclosed Cas9 fusion proteins and gRNA into extracellular vesicles (EVs). The cells include any cell suitable for recombinant protein expression, for example, cells comprising a genetic construct expressing or capable of expressing a fusion protein disclosed herein (e.g., cells that have been transformed with one or more vectors described herein, or cells having genomic modifications, for example, those that express a protein provided herein from an allele that has been incorporated in the cell's genome). Methods for transforming cells, genetically modifying cells, and expressing genes and proteins in such cells are well known in the art, and include those provided by, for example, Green and Sambrook, Molecular Cloning: A Laboratory Manual (4th ed., Cold Spring Harbor Laboratory Press, Cold Spring Harbor, N.Y. (2012)) and Friedman and Rossi, Gene Transfer: Delivery and Expression of DNA and RNA, A Laboratory Manual (1st ed., Cold Spring Harbor Laboratory Press, Cold Spring Harbor, N.Y. (2006)).

Some aspects of this disclosure provide kits comprising a polynucleotide encoding a Cas9 fusion protein provided herein. In some embodiments, the kit comprises a vector for recombinant protein expression, wherein the vector comprises a polynucleotide encoding any of the proteins provided herein. In some embodiments, the kit comprises a cell (e.g., any cell suitable for expressing Cas9 fusions proteins, such as bacterial, yeast, or mammalian cells) that comprises a genetic construct for expressing any of the proteins provided herein. In some embodiments, any of the kits provided herein further comprise one or more gRNAs and/or vectors for expressing one or more gRNAs. In some embodiments, the kit comprises an excipient and instructions for contacting the nuclease and/or recombinase with the excipient to generate a composition suitable for contacting a nucleic acid with the nuclease and/or recombinase such that hybridization to and cleavage and/or recombination of a target nucleic acid occurs. In some embodiments, the composition is suitable for delivering a Cas9 protein to a cell. In some embodiments, the composition is suitable for delivering a Cas9 protein to a subject. In some embodiments, the excipient is a pharmaceutically acceptable excipient.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Fatty Acylation Regulates the Encapsulation of Src Family Kinases into Extracellular Vesicles

Protein N-myristoylation is a co/post-translational modification that results in covalent attachment of the myristoyl group (14-carbon saturated fatty acyl) to the N-terminus of a target protein (Wright M H, et al. J Chem Biol. 2010 3:19-35). A consensus sequence of Met-Gly-x-x-x-Ser/Thr (SEQ ID NO:3) at the N-terminus is essential for the N-myristoylation process. Myristoylation modification occurs after the first methionine is removed by methionine aminopeptidase during protein translation, and Gly2 is the site of the attachment of the myristoyl group (Udenwobele D I, et al. 2017 8:751). A panel of proteins have been reported to be myristoylated in mammalian cells (Resh M D. Biochimica et biophysica acta. 1999 1451:1-16). Myristoylation allows these proteins to participate in a variety of molecular functions such as cellular localization, cell signaling, and cell-cell communication (Kim S, et al. J Biol Chem. 2017; Casey P J. Science. 1995 268:221). These activities can subsequently regulate the proliferation of cancer cells, tumor progression, immune response, and other biological functions (Udenwobele D I, et al. 2017 8:751; Kim S, et al. Cancer Res. 2017 77:6950-62). Targeting protein myristoylation is a potential therapeutic approach for the treatment of cancer progression (Kim S, et al. Cancer Res. 2017 77:6950-62; Li Q, et al. J Biol Chem. 2018 293:6434-48; Sulejmani E, et al. Oncoscience. 2018 5:3-5).

Src family kinases (SFKs), a group of non-receptor tyrosine kinases, are among the identified myristoylated proteins (Martin G S. Nat Rev Mol Cell Biol. 2001 2:467-75). All SFK members are composed of an N-terminal Src Homology (SH) 4 domain controlling membrane association via myristoylation and, depending on the SFK, palmitoylation. For example, both Src and Fyn kinase are N-myristoylated, but Fyn kinase is also palmitoylated at cysteine residues at sites 3 and 6 in the N-terminus (Resh M D. Biochimica et biophysica acta. 1999 1451:1-16; Cai H, et al. Proc Natl Acad Sci USA. 2011 108:6579-84; Resh M D. Cell. 1994 76:411-3). SFKs also contain SH3, SH2, tyrosine kinase SH1 domains, and a short C-terminal tail containing an autoinhibitory phosphorylation site, such as Tyr529 in human Src kinase (Xu W, et al. Nature. 1997 385:595; Sicheri F, et al. Curr Opin Cell Biol. 1997 7:777-85). The expression and activity of Src kinase is highly up-regulated in various cancers including aggressive prostate cancer (Guo Z, et al. Cancer Cell. 2006 10:309-19; Drake J M, et al. Proc Natl Acad Sci USA. 2013 110:E4762-9), which is associated with short life expectancy and a high probability of distant metastasis (Fizazi K. Ann Oncol. 2007 18:1765-73; Erpel T, et al. Curr Opin Cell Biol. 1995 7:176-82; Parsons J T, et al. Curr Opin Cell Biol. 1997 9:187-92; Tatarov O, et al. Clin Cancer Res. 2009 15:3540-9; Irby R B, et al. Oncogene. 2000 19:5636). Differential patterns of myristoylation and/or palmitoylation of SFKs determines their cellular localization (Kim S, et al. J Biol Chem. 2017; Patwardhan P, et al. Mol Cell Biol. 2010 30:4094-107), the interaction of Src kinase with androgen receptor (Kim S, et al. Cancer Res. 2017 77:6950-62), intracellular trafficking (Sato I, et al. J Cell Sci. 2009 122:965-75), and subsequently their kinase activity and transformation potential (Kim S, et al. J Biol Chem. 2017; Cai H, et al. Proc Natl Acad Sci USA. 2011 108:6579-84; Patwardhan P, et al. Mol Cell Biol. 2010 30:4094-107; Oneyama C, et al. 2008 30:426-36; Oneyama C, et al. Mol Cell Biol. 2009 29:6462-72). Exogenous myristate in a high-fat diet can regulate Src kinase levels at the cell membrane via myristoylation, and accelerate Src-mediated oncogenic potential and tumorigenesis (Kim S, et al. J Biol Chem. 2017; Kim S, et al. Cancer Res. 2017 77:6950-62).

Extracellular vesicles (EVs) are nanovesicles with a diameter of 30-150 nm secreted from almost all cell types (Kowal J, et al. Curr Opin Cell Biol. 2014 29:116-25). EVs mediate cell-to-cell communication through the transfer of lipids, proteins, mRNAs, microRNAs, and other exosomal contents (Villarroya-Beltri C, et al. Sem Cell Biol. 2014 28:3-13; Simons M, et al. Curr Opin Cell Biol. 2009 21:575-81). The EVs-mediated cellular interaction can facilitate the dissemination of diseases, promote tumor progression and metastasis, and escape the immune system (Hoshino A, et al. Nature. 2015 527:329-35; Kahlert C, et al. J Mol Med. 2013 91:431-7; Skog J, et al. Nat Cell Biol. 2008 10:1470-6; Abusamra A J, et al. Blood Cells Mol Dis. 2005 35:169-73). EVs are generated through cell exocytosis originated from the fusion of multi-vesicular bodies with the plasma membrane (Thery C, et al. Nat Rev Immunol. 2002 2:569-79; Colombo M, et al. Annu Rev Cell Dev Biol. 2014 30:255-89; Keller S, et al. Immunol Lett. 2006 107:102-8). Here, we study how fatty acylation modulates the encapsulation of proteins into EVs. As disclosed herein, the encapsulation of SFK members into EVs is regulated by myristoylation, palmitoylation, and Src kinase activity, and the encapsulation process involves the syntenin-ESCRT mediated biogenesis pathway.

Materials and Methods

Plasmids

Lentiviral vectors expressing Src(WT), Src(G2A), Src(Y529F), Src(Y529F/G2A), Src(S3C/S6C), Fyn(WT), Fyn (G2A), or Fyn (C3S/C6S) were cloned into the FUCRW parental lentiviral vector as previously reported (Kim S, et al. J Biol Chem. 2017; Cai H, et al. Proc Natl Acad Sci USA. 2011 108:6579-84). Knockdown of Src kinase by shRNA was created in a previous study (Kim S, et al. Cancer Res. 2017 77:6950-62). Two lentiviral vectors expressing shRNA-TSG101 were obtained from Sigma Aldrich. The sequence of shRNA-TSG101-1 was 5′-CCGGACTGGACACATACCCATATAACTCGAGTTATATGGGTATGTGTCCAGTTTTTTG-3′ (SEQ ID NO:7) and the sequence of shRNA-TSG101-2 was 5′-CCGGGCCTTATAGAGGTAATACATACTCGAGTATGTATTACCTCTATAAGGCTTTTG-3′ (SEQ ID NO:8). The lentivirus were generated from these lentiviral vectors to create stable cell lines. The lentiviral production followed the guidelines of the University of Georgia.

Cell Lines

SYF1 (Src^{-/-}Fyn^{-/-}Yes^{-/-}), 3T3, and human prostate cancer cell lines including DU145, PC3, 22Rv1, and LNCaP were purchased from American Type Culture Collection (ATCC). The cells were grown in the medium recommended by ATCC. Mycoplasma contamination was examined periodically. The cells were used up to 20 passages.

Isolation of EVs and Characterization

To isolate EVs from the cell culture medium, the cell lines were grown in ATCC recommended medium in a 150-mm petri-dish. After reaching 90% confluence, the medium was replaced with fresh medium containing 5% exosome-free FBS (Life Technology Inc.), and grown in 5% CO₂ 37° C. incubator for another 24 h. The conditioned medium was collected for the EVs isolation. Specifically, the conditioned medium was repeatedly centrifuged at 4° C. at 300×g for 10 min, 2,000×g for 10 min, and 10,000×g for 30 min to remove live cells, dead cells, and cell debris, respectively. The supernatant was further ultra-centrifugated with 100,000×g at 4° C. for 90 min. The EVs pellet was re-suspended in 1×PBS to wash out the residual medium, and re-centrifugated at 100,000×g at 4° C. for 90 min. The pelleted EVs were re-suspended either in RIPA buffer for protein analysis or 1×PBS for Dynamic Light Scattering (DLS) analysis. The size, zeta potential, and concentration of EVs were measured by nanoparticle tracking analysis (NTA, Particle Metrix, Germany) with ZetaView software for data record and analysis.

Protein Concentration Determination

The protein concentration of EVs and cell lysates was determined by detergent compatible (DC) protein assay (Bio-Rad Laboratories). The total cell lysates (TCL) and EVs were dissolved in RIPA buffer [50 mM Tris-base (pH 7.4), 1% NP-40, 0.50% sodium deoxycholate, 0.1% SDS, 150 mM NaCl, 2 mM EDTA and protease inhibitor (1×)] and the manufacturer's protocol was followed.

Antibodies and Western Blotting Analysis

The total cell lysate and EVs dissolved in RIPA buffer were subjected to the standard immunoblotting analysis. The following antibodies were used: rabbit anti-Src (Cat #: 2109), rabbit anti-calnexin (Cat #: 2679), rabbit anti-CD-9 (Cat #: 13403 for human species, Cat #: 2118 for mouse species), rabbit anti-GAPDH (Cat #: 13403), rabbit anti-Fyn (Cat #: 4023), and rabbit anti-FAK (Cat #: 13009), rabbit CD81 (Cat #: 10037) were purchased from Cell Signaling Technology; rabbit anti-RFP (Cat #: 600-401-379, Rockland Inc), rabbit anti-AR (Cat #: sc-816, Santa Cruz Biotechnology), and secondary Antibody anti-rabbit IgG HRP (Cat #: 7074, Cell Signaling Technology) were used according to manufactory's recommended dilution. The band intensity was quantified by Image J software.

Determination of Myristoylated Src Kinase by Click Chemistry

Cells expressing Src kinase were grown until 90% confluence in EMEM medium with 5% FBS. The medium was replaced with EMEM medium containing exosome-free FBS and 50 μM of myristic acid-azide (an analog of myristic acid) and the cells were grown for another 24 h. The conditioned medium was collected and used for EVs isolation as described above. The cells or EVs were lysed in M-PER buffer (Thermo Scientific) containing protease inhibitors and phosphatase inhibitors. The cell lysates or EVs lysate (10 μg protein) were added to a working solution containing biotin-alkyne (0.1 mM), CuSO₄ (1 mM), TCEP (1 mM) and TBTA (0.1 mM) and incubated at room temperature for 1 h. After the Click reaction, the samples were mixed with loading dye and boiled at 95° C. for 5 min. The lysates were subjected to SDS-PAGE and transferred to a nitrocellulose membrane. After blocking with 5% milk overnight, the membrane was incubated with High Sensitivity Streptavidin-HRP (catalog No. 21130, ThermoFisher Scientific) at room temperature for 1 h. Myristoylated proteins (e.g., myristoylated Src kinase) were detected by ECL.

Lipid Raft Disruption

PC3 and DU145 cells were grown overnight. The medium was replaced with the same growth medium but containing EVs/exosome-free FBS with DMSO (control) or Filipin III (0-1 μM) for 24 h to disrupt lipid rafts. The EVs were isolated from the conditioned medium by sequential centrifugation as described above. The isolated EVs and cells were lysed with RIPA buffer for immunoblotting analysis.

Xenograft Tumors and EVs Isolation and Characterization from the Plasma

All animal studies were approved by the Institutional Animal Care and Use Committee (IACUC) of the University of Georgia. To establish the xenograft tumors, DU145 cells were transduced with control, Src(Y529F), or Src(Y529F/G2A) by lentiviral infection. Male SCID mice at the age of 8-10 weeks were randomly divided into 4 groups. The transduced cells were implanted to the sub-renal capsule of SCID mice. The mice were routinely examined and euthanized after 5-weeks incubation. The xenograft tumors and the blood from the host were collected for further analysis.

After centrifugation at 2,000×g for 10 min, the supernatant from the collected blood samples was collected. The plasma EVs were isolated by the Exoquick kit according to manufacturer's instructions (Cat #: EXOQ5A-1, System Biosciences). The isolated EVs were re-suspended in PBS buffer for characterization of size and zeta potential by DLS with zetasizer (Malvern, USA). The isolated EVs were lysed in RIPA buffer for Western blot analysis.

Identification of Myristoylated Proteins by Bioinformatics

To identify potential myristoylated proteins in the mammalian genome, the Uniprot database was accessed and searched using the keyword “myristate” and the filters “Reviewed” and “Homo sapiens”. 194 results were recovered and downloaded for further analysis. The sequences of proteins were analyzed and any protein sequences lacking a glycine at the second position were removed from the list. The remaining 182 proteins were checked together with the EVs data provided from the NCI-60 cell lines, and grouped by the number of times each protein appeared in EVs, with 60 being the highest and 0 being the lowest (Hurwitz S N, et al. Oncotarget. 2016 7:86999; Khoury G A, et al. Sci Rep. 2011 1:90; Consortium U. Nucleic Acids Res. 2016 45:D158-D69).

A literature review focusing on the proteomic analysis of EVs uncovered three published studies on thymic, breast milk, and urine EVs: “Characterization of human thymic exosomes”, “Comprehensive Proteomic Analysis of Human Milk-derived Extracellular Vesicles Unveils a Novel Functional Proteome Distinct from Other Milk Components”, and “Proteomic analysis of urine exosomes by multidimensional protein identification technology (MudPIT)” (Wang Z, et al. Proteomics. 2012 12:329-38; van Herwijnen M J, et al. Mol Cell Proteomics. 2016 15:3412-23; Skogberg G, et al. PloS one. 2013 8:e67554). The 182 proteins taken from the Uniprot database were checked against the EVs data from each of the three studies, and their appearances in each of the three studies were recorded.

Statistical Analysis

The data are presented as mean±SEM (standard error of the mean). All the data with more than two groups were analyzed by one-way ANOVA with a post hoc Tukey test in GraphPad Prism software, and two values were compared by an unpaired student t-test. * p<0.05; ** p<0.01; *** p<0.001; NS: not significant.

Haemotoxylin and Eosin (H&E) Staining

The tissue samples were fixed with PBS buffered 10% formaldehyde. The samples were paraffin-embedded and sectioned in Leica RM2235 Rotary Microtomy to 4 μm thickness and mounted on microscope slides (catalog No. 12-550-15, Fisher Scientific). Paraffin embedded sections were treated as follows: 100% xylene to de-paraffin for 5 min (3×), 100% ethanol to rehydrate for 2 min (2×), 95% ethanol for 2 min (2×), 75% ethanol for 2 min (2×), and then rinsed thoroughly by distilled water (3×). The sections were stained in Ehrlich's Hematoxylin for 5 min and washed with distilled water (3×), followed by 5-6 quick dips in acid alcohol (0.3%) to differentiate and wash thoroughly with distilled water (3×). The tissue sections were dipped into Scott's Tap Solution for 2 min and rinsed thoroughly with distilled water (3×) followed by counterstain in Eosin solution for 2 min and washed with distilled water (3×), followed by dehydration in 95% alcohol for 5 dips (2×) and 100% alcohol for 5 dips (2×). After xylene clearing for 1 min (3×), tissue sections were mounted with a coverslip in the mounting medium.

Immunohistochemistry (IHC) Staining

4 μm thickness of tissue section on a microscope slide was baked for 60 min at 65° C., and de-paraffined in 100% xylene for 5 min (2×), dehydrated in 100% ethanol for 5 min (2×), 95% ethanol for 5 min (2×), 70% ethanol for 5 min. After washing with PBS for 10 min (3×), the tissue slides were cooked in 0.01 M citrate buffer (pH 6.0) in a steamer cooker at a microwave with 60% power for 15 min and 10% power. After cooling, tissue slides were washed with PBS for 10 min (2×). The tissues were circled with a PAP Pen liquid blocker (Part #6505, Newcomer Supply). 300 μL of 0.3% H₂O₂ in distilled water was added into each tissue spot for 5-10 min and then washed with PBS for 10 min (3×). The tissues were blocked in 2.5% goat serum in PBS for 1 h at room temperature, and then incubated with primary Src antibody (1:250) in PBST overnight at 4° C. The tissue slides were washed with PBST for 10 min (3×), and then incubated with secondary antibody (Cat: M7401) in PBST at room temperature for 1 h. After washing with PBS for 10 min (×3), the tissues slides were incubated with DAB solution (catalog No. SK-4100) for development. As soon as brown color appeared under a microscope, the reaction was stopped by dipping the slide into distilled water. The time to develop for control and treatment was kept the same. The tissue slides were stained in Hematoxylin for 1 min and washed with distilled water (×3), then immersed in NaHCO₃ solution for 3 min and washed with distilled water (×3). The tissue slides were again dehydrated by treating samples in a series of alcohol solutions (75%, 95%, 100% ethanol for 5 min×2), and then air dried for 10 min. After treating with xylene for 5 min (×2), the tissue sections were air dried for 10 min, and mounted with the mounting medium and coverslip.

Detection of Palmitoylation by Click Chemistry

Cells expressing Src kinase were grown until 90% confluence in the EMEM medium with 5% PBS. The medium was replaced with the EMEM medium containing exosome-free FBS and 50 μM of myristic acid-azide (an analog of myristic acid) and the cells were grown for another 24 h. The conditioned medium was collected and used for extracellular vesicles (EVs) isolation by the ultracentrifuge method. The cells or EVs were lysed in M-PER buffer (Thermo Scientific) containing protease inhibitors and phosphatase inhibitors. The cell lysates or EVs lysate (10 μg protein) were added into a working solution containing biotin-alkyne (0.1 mM), CuSO₄ (1 mM), TCEP (1 mM) and TBTA (0.1 mM) and incubated at room temperature for 1 h. After the Click reaction, the samples were mixed with loading dye and boiled at 95° C. for 5 min. The lysates were subjected to SDS-PAGE and transferred to a nitrocellulose membrane. After blocking with 5% milk overnight, the membrane was incubated with High Sensitivity Streptavidin-HRP (catalog No. 21130, ThermoFisher Scientific) at room temperature for 1 h. Myristoylated proteins (e.g., myristoylated Src kinase) were detected by ECL.

Results

The appearance frequency of myristoylated proteins is elevated in extracellular vesicles.

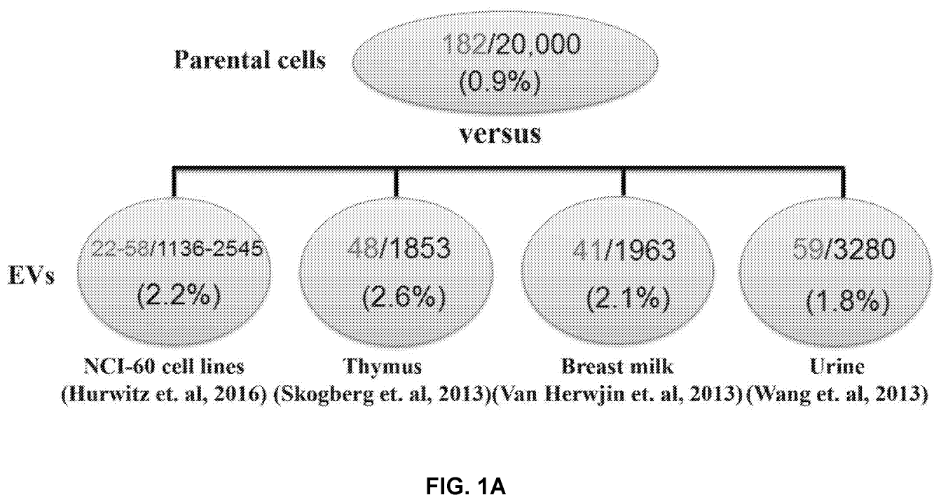

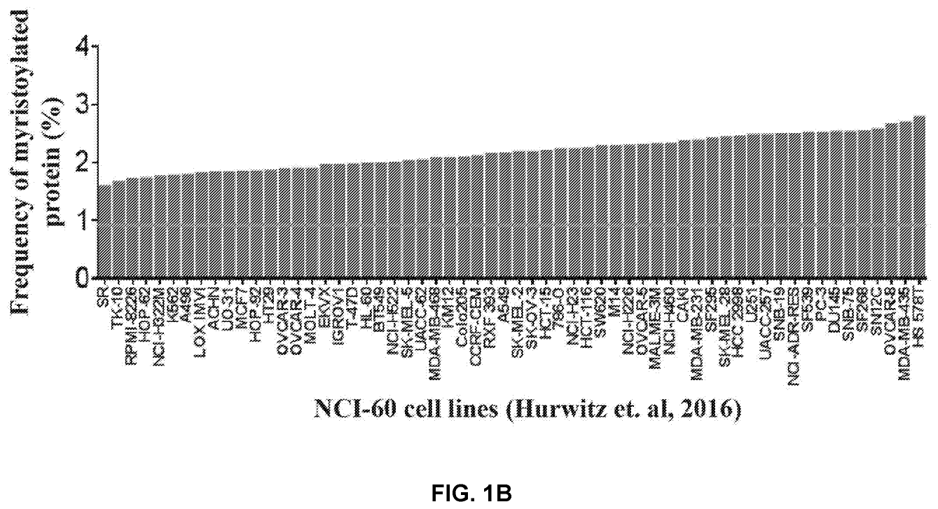

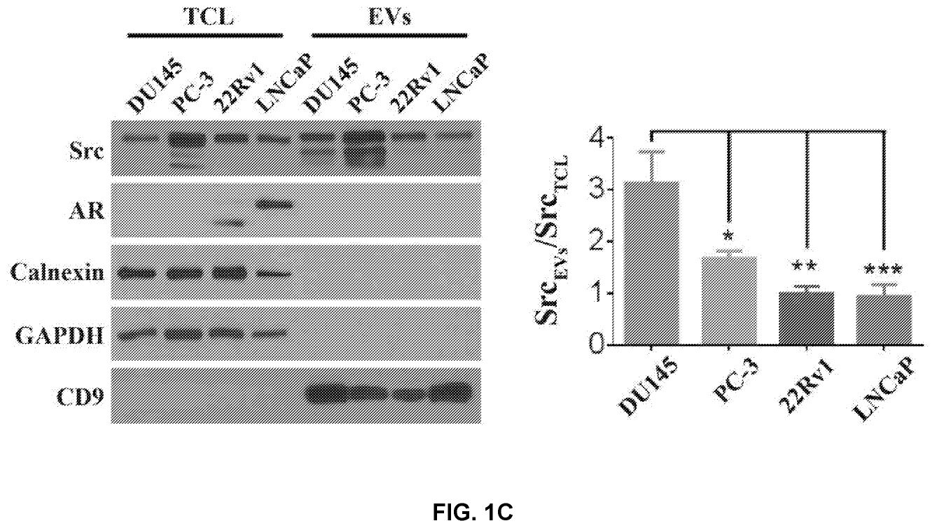

The N-terminal glycine (Gly2) is required for protein myristoylation after removal of methionine by methionine aminopeptidase. By searching the mammalian genome for proteins that fit the essential myristoylation requirement, 182 potentially myristoylated proteins were identified (Hurwitz S N, et al. Oncotarget. 2016 7:86999; Khoury G A, et al. Sci Rep. 2011 1:90; Consortium U. Nucleic Acids Res. 2016 45:D158-D69). Given a total of about 20,000 proteins in a mammalian cell, the percentage of myristoylated proteins accounts for about 0.9% of the mammalian genome (FIG. 1A). Based on the proteomics study (Hurwitz S N, et al. Oncotarget. 2016 7:86999), the number of myristoylated proteins in extracellular vesicles (EVs) represented 2.2% of total identified proteins in EVs of 60 cancer cell lines (FIG. 1A and Tables 1-2). The appearance frequency of myristoylated proteins detected in EVs ranged from 1.6-2.8% of total proteins in EVs of each individual cancer cell line, which was significantly higher than 0.9% of myristoylated proteins in a cell (FIG. 1B). The appearance frequency of myristoylated proteins in EVs was also elevated in three normal tissues. Specifically, 48, 41, and 59 myristoylated proteins were identified from 1853 proteins of EVs in thymus, 1963 in breast milk, and 3280 in urine, respectively, which represented 2.6%, 2.1%, and 1.8% of total identified proteins in EVs (FIG. 1A, Tables 3-5) (Wang Z, et al. Proteomics. 2012 12:329-38; van Herwijnen M J, et al. Mol Cell Proteomics. 2016 15:3412-23; Skogberg G, et al. PloS one. 2013 8:e67554). Collectively, the data suggest that myristoylated proteins occur more frequently in EVs in vitro and in vivo.

TABLE 1
182 potential myristoylated proteins in mammalian cells and their appearance frequency
in extracellular vesicles of 60 cancer cell lines

| | | | Appearance |
|---|---|---|---|
| | | | frequency in |
| Protein | | | 60 cancer |
| ID | Gene Name | N-terminus sequence | cell lines |
| P84077 | ARF1 | MGNIFANLFKGLFGKKEMRILMVGLDAAGK (SEQ ID NO: 9) | 60 |
| P18085 | ARF4 ARF2 | MGLTISSLFSRLFGKKQMRILMVGLDAAGK (SEQ ID NO: 10) | 60 |
| P62330 | ARF6 | MGKVLSKIFGNKEMWILMLGLDAAGKTTIL (SEQ ID NO: 11) | 60 |
| P04899 | GNAI2 GNAI2B | MGCTVSAEDKAAAERSKMIDKNLREDGEKA (SEQ ID NO: 12) | 60 |
| P08754 | GNAI3 | MGCTLSAEDKAAVERSKMIDRNLREDGEKA (SEQ ID NO: 13) | 60 |
| P62241 | RPS8 OK/SW-cl.83 | MGISRDNWHKRRKTGGKRKPYHKKRKYELG (SEQ ID NO: 14) | 60 |
| Q96TA1 | FAM129B C9orf88 | MGDVLSTHLDDARRQHIAEKTGKILTEFLQ (SEQ ID NO: 15) | 58 |
| Q6IAA8 | LAMTOR1 C11orf59 PDRO | MGCCYSSENEDSDQDREERKLLLDPSSPPT (SEQ ID NO: 16) | 57 |
| | PP7157 | | |
| Q14254 | FLOT2 ESA1 M17S1 | MGNCHTVGPNEALVVSGGCCGSDYKQYVFG (SEQ ID NO: 17) | 56 |
| P84085 | ARF5 | MGLTVSALFSRIFGKKQMRILMVGLDAAGK (SEQ ID NO: 18) | 54 |
| P61313 | RPL15 EC45 TCBAP0781 | MGAYKYIQELWRKKQSDVMRFLLRVRCWQY (SEQ ID NO: 19) | 54 |
| P07947 | YES1 YES | MGCIKSKENKSPAIKYRPENTPEPVSTSVS (SEQ ID NO: 20) | 54 |
| Q9NUQ9 | FAM49B BM-009 | MGNLLKVLTCTDLEQGPNFFLDFENAQPTE (SEQ ID NO: 21) | 52 |
| Q9H4G4 | GLIPR2 C9orf19 GAPR1 | MGKSASKQFHNEVLKAHNEYRQKHGVPPLK (SEQ ID NO: 22) | 52 |
| P63096 | GNAI1 | MGCTLSAEDKAAVERSKMIDRNLREDGEKA (SEQ ID NO: 23) | 52 |
| P36405 | ARL3 ARFL3 | MGLLSILRKLKSAPDQEVRILLLGLDNAGK (SEQ ID NO: 24) | 51 |
| P36404 | ARL2 | MGLLTILKKMKQKERELRLLMLGLDNAGKT (SEQ ID NO: 25) | 50 |
| Q96FZ7 | CHMP6 VPS20 | MGNLFGRKKQSRVTEQDKAILQLKQQRDKL (SEQ ID NO: 26) | 50 |
| Q99653 | CHP1 CHP | MGSRASTLLRDEELEEIKKETGFSHSQITR (SEQ ID NO: 27) | 50 |
| Q8WWI5 | SLC44A1 CD92 CDW92 CTL1 | MGCCSSASSAAQSSKREWKPLEDRSCTDIP (SEQ ID NO: 28) | 49 |
| P07948 | LYN JTK8 | MGCIKSKGKDSLSDDGVDLKTQPVRNTERT (SEQ ID NO: 29) | 47 |
| P49006 | MARCKSL1 MLP MRP | MGSQSSKAPRGDVTAEEAAGASPAKANGQE (SEQ ID NO: 30) | 47 |
| O75695 | RP2 | MGCFFSKRRKADKESRPENEEERPKQYSWD (SEQ ID NO: 31) | 47 |
| P29966 | MARCKS MACS PRKCSL | MGAQFSKTAAKGEAAAERPGEAAVASSPSK (SEQ ID NO: 32) | 46 |
| Q8N9N7 | LRRC57 | MGNSALRAHVETAQKTGVFQLKDRGLTEFP (SEQ ID NO: 33) | 45 |
| P37235 | HPCAL1 BDR1 | MGKQNSKLRPEVLQDLRENTEFTDHELQEW (SEQ ID NO: 34) | 44 |
| P00387 | CYB5R3 DIA1 | MGAQLSTLGHMVLFPVWFLYSLLMKLFQRS (SEQ ID NO: 35) | 43 |
| Q9NRX5 | SERINC1 KIAA1253 TDE1L | MGSVLGLCSMASWIPCLCGSAPCLLCRCCP (SEQ ID NO: 36) | 42 |
| | TDE2 UNQ396/PRO732 | | |
| P12931 | SRC SRC1 | MGSNKSKPKDASQRRRSLEPAENVHGAGGG (SEQ ID NO: 37) | 42 |
| P40616 | ARL1 | MGGFFSSIFSSLFGTREMRILILGLDGAGK (SEQ ID NO: 38) | 40 |
| P80723 | BASP1 NAP22 | MGGKLSKKKKGYNVNDEKAKEKDKKAEGAA (SEQ ID NO: 39) | 40 |
| Q9NX63 | CHCHD3 MIC19 MINOS3 | MGGTTSTRRVTFEADENENITVVKGIRLSE (SEQ ID NO: 40) | 39 |
| Q96PY5 | FMNL2 FHOD2 KIAA1902 | MGNAGSMDSQQTDFRAHNVPLKLPMPEPGE (SEQ ID NO: 41) | 38 |
| P62166 | NCS1 FLUP FREQ | MGKSNSKLKPEVVEELTRKTYFTEKEVQQW (SEQ ID NO: 42) | 38 |
| Q9BZQ8 | FAM 129A C1orf24 NIBAN | MGGSASSQLDEGKCAYIRGKTEAAIKNFSP (SEQ ID NO: 43) | 37 |
| | GIG39 | | |
| Q8NHG7 | SVIP | MGLCFPCPGESAPPTPDLEEKRAKLAEAAE (SEQ ID NO: 44) | 37 |
| Q9Y3E7 | CHMP3 CGI149 NEDF VP524 | MGLFGKTQEKPPKELVNEWSLKIRKEMRVV (SEQ ID NO: 45) | 35 |
| | CGI-149 | | |
| Q99828 | CIB1 CIB KIP PRKDCIP | MGGSGSRLSKELLAEYQDLTFLTKQEILLA (SEQ ID NO: 46) | 32 |
| P17612 | PRKACA PKACA | MGNAAAAKKGSEQESVKEFLAKAKEDFLKK (SEQ ID NO: 47) | 31 |
| Q8ND76 | CCNY C10orf9 CBCP1 CFP1 | MGNTTSCCVSSSPKLRRNAHSRLESYRPDT (SEQ ID NO: 48) | 30 |
| Q9H8Y8 | GORASP2 GOLPH6 | MGSSQSVEIPGGGTEGYHVLRVQENSPGHR (SEQ ID NO: 49) | 29 |
| Q99570 | PIK3R4 VPS15 | MGNQLAGIAPSQILSVESYFSDIHDFEYDK (SEQ ID NO: 50) | 28 |
| Q14699 | RFTN1 KIAA0084 MIG2 | MGCGLNKLEKRDEKRPGNIYSTLKRPQVET (SEQ ID NO: 51) | 25 |
| Q7L014 | DDX46 KIAA0801 | MGRESRHYRKRSASRGRSGSRSRSRSPSDK (SEQ ID NO: 52) | 24 |
| O60936 | NOL3 ARC NOP | MGNAQERPSETIDRERKRLVETLQADSGLL (SEQ ID NO: 53) | 24 |
| P08473 | MME EPN | MGKSESQMDITDINTPKPKKKQRWTPLEIS (SEQ ID NO: 54) | 22 |
| P22694 | PRKACB | MGNAATAKKGSEVESVKEFLAKAKEDFLKK (SEQ ID NO: 55) | 22 |
| Q8IV36 | HID1 C17orf28 DMC1 | MGSTDSKLNFRKAVIQLTTKTQPVEATDDA (SEQ ID NO: 56) | 21 |
| Q8IVF7 | FMNL3 FHOD3 FRL2 | MGNLESAEGVPGEPPSVPLLLPPGKMPMPE (SEQ ID NO: 57) | 19 |
| | KIAA2014 WBP3 | | |
| O15355 | PPM1G PPM1C | MGAYLSQPNTVKCSGDGVGAPRLPLPYGFS (SEQ ID NO: 58) | 19 |
| Q9NUM4 | TMEM106B | MGKSLSHLPLHSSKEDAYDGVTSENMRNGL (SEQ ID NO: 59) | 19 |
| P09471 | GNAO1 | MGCTLSAEERAALERSKAIEKNLKEDGISA (SEQ ID NO: 60) | 17 |
| O75896 | TUSC2 C3orf11 FUS1 LGCC | MGASGSKARGLWPFASAAGGGGSEAAGAEQ (SEQ ID NO: 61) | 16 |
| | PDAP2 | | |
| Q9NS886 | LANCL2 GPR69B TASP | MGETMSKRLKLHLGGEAEMEERAFVNPFPD (SEQ ID NO: 62) | 15 |
| Q02952 | AKAP12 AKAP250 | MGAGSSTEQRSPEQPPEGSSTPAEPEPSGG (SEQ ID NO: 63) | 13 |
| P06239 | LCK | MGCGCSSHPEDDWMENIDVCENCHYPIVPL (SEQ ID NO: 64) | 11 |
| P27216 | ANXA13 ANX13 | MGNRHAKASSPQGFDVDRDAKKLNKACKGM (SEQ ID NO: 65) | 10 |
| P06241 | FYN | MGCVQCKDKEATKLTEERDGSLNQSSGYRY (SEQ ID NO: 66) | 10 |
| O00461 | GOLIM4 GIMPC GOLPH4 | MGNGMCSRKQKRIFQTLLLLTVVFGFLYGA (SEQ ID NO: 67) | 9 |
| | GPP130 | | |
| P63098 | PPP3R1 CNA2 CNB | MGNEASYPLEMCSHFDADEIKRLGKRFKKL (SEQ ID NO: 68) | 9 |
| P62760 | VSNL1 VISL1 | MGKQNSKLAPEVMEDLVKSTEFNEHELKQW (SEQ ID NO: 69) | 9 |
| Q8IWE4 | DCUN1D3 SCCRO3 | MGQCVTKCKNPSSTLGSKNGDREPSNKSHS (SEQ ID NO: 70) | 8 |
| P29728 | OAS2 | MGNGESQLSSVPAQKLGWFIQEYLKPYEEC (SEQ ID NO: 71) | 8 |
| O75688 | PPM1B PP2CB | MGAFLDKPKTEKHNAHGAGNGLRYGLSSMQ (SEQ ID NO: 72) | 7 |
| P56559 | ARL4C ARL7 | MGNISSNISAFQSLHIVMLGLDSAGKTTVL (SEQ ID NO: 73) | 6 |
| Q86UY6 | NAA40 NAT11 PATT1 | MGRKSSKAKEKKQKRLEERAAMDAVCAKVD (SEQ ID NO: 74) | 6 |
| Q9ULE6 | PALD1 KIAA1274 PALD | MGTTASTAQQTVSAGTPFEGLQGSGTMDSR (SEQ ID NO: 75) | 6 |
| O43149 | ZZEF1 KIAA0399 | MGNAPSHSSEDEAAAAGGEGWGPHQDWAAV (SEQ ID NO: 76) | 6 |
| Q9BRQ8 | AIFM2 AMID PRG3 | MGSQVSVESGALHVVIVGGGFGGIAAASQL (SEQ ID NO: 77) | 5 |
| Q9YNA8 | ERVK-19 | MGQTKSKIKSKYASYLSFIKILLKRGGVKV (SEQ ID NO: 78) | 5 |
| Q9C0E8 | LNPK KIAA1715 LNP | MGGLFSRWRTKPSTVEVLESIDKEIQALEE (SEQ ID NO: 79) | 5 |
| Q96BS2 | TESC CHP3 | MGAAHSASEEVRELEGKTGFSSDQIEQLHR (SEQ ID NO: 80) | 5 |
| Q9Y250 | LZTS1 FEZ1 | MGSVSSLISGHSFHSKHCRASQYKLRKSSH (SEQ ID NO: 81) | 4 |
| Q969G9 | NKD1 NKD PP7246 | MGKLHSKPAAVCKRRESPEGDSFAVSAAWA (SEQ ID NO: 82) | 4 |
| Q9Y3C5 | RNF11 CGI-123 | MGNCLKSPTSDDISLLHESQSDRASFGEGT (SEQ ID NO: 84) | 4 |
| Q8NHG8 | ZNRF2 RNF202 | MGAKQSGPAAANGRTRAYSGSDLPSSSSGG (SEQ ID NO: 85) | 4 |
| O15121 | DEGS1 DES1 MLD MIG15 | MGSRVSREDFEWVYTDQPHADRRREILAKY (SEQ ID NO: 86) | 3 |
| Q8WU20 | FRS2 | MGSCCSCPDKDTVPDNHRNKFKVINVDDDG (SEQ ID NO: 87) | 3 |
| P08631 | HCK | MGGRSSCEDPGCPRDEERAPRMGCMKSKFL (SEQ ID NO: 88) | 3 |
| Q9P032 | NDUFAF4 C6orf66 HRPAP20 | MGALVIRGIRNFNLENRAEREISKMKPSVA (SEQ ID NO: 89) | 3 |
| | HSPC125 My013 | | |
| P17568 | NDUFB7 | MGAHLVRRYLGDASVEPDPLQMPTFPPDYG (SEQ ID NO: 90) | 3 |
| P40617 | ARL4A ARL4 | MGNGLSDQTSILSNLPSFQSFHIVILGLDC (SEQ ID NO: 91) | 2 |
| Q9H0F7 | ARL6 BBS3 | MGLLDRLSVLLGLKKKEVHVLCLGLDNSGK (SEQ ID NO: 92) | 2 |
| Q9BSF0 | C2orf88 | MGCMKSKQTFPFPTIYEGEKQHESEEPFMP (SEQ ID NO: 93) | 2 |
| Q9BRQ6 | CHCHD6 CHCM1 MIC25 | MGSTESSEGRRVSFGVDEEERVRVLQGVRL (SEQ ID NO: 94) | 2 |
| Q7L9B9 | EEPD1 KIAA1706 | MGSTLGCHRSIPRDPSDLSHSRKFSAACNF (SEQ ID NO: 95) | 2 |
| P63130 | ERVK-7 | MGQTKSKIKSKYASYLSFIKILLKRGGVKV (SEQ ID NO: 96) | 2 |
| P19086 | GNAZ | MGCRQSSEEKEAARRSRRIDRHLRSESQRQ (SEQ ID NO: 97) | 2 |
| Q9Y6M0 | PSMC1 | MGARGALLLALLLARAGLRKPESQEAAPLS (SEQ ID NO: 98) | 2 |
| P19087 | GNAT2 GNATC | MGSGASAEDKELAKRSKELEKKLQEDADKE (SEQ ID NO: 99) | 1 |
| A8MTJ3 | GNAT3 | MGSGISSESKESAKRSKELEKKLQEDAERD (SEQ ID NO: 100) | 1 |
| O60291 | MGRN1 KIAA0544 RNF156 | MGSILSRRIAGVEDIDIQANSAYRYPPKSG (SEQ ID NO: 101) | 1 |
| Q6BDI9 | REP15 | MGQKASQQLALKDSKEVPVVCEVVSEAIVH (SEQ ID NO: 102) | 1 |
| Q52LD8 | RFTN2 C2orf11 | MGCGLRKLEDPDDSSPGKIFSTLKRPQVET (SEQ ID NO: 103) | 1 |
| Q8IZE3 | SCYL3 PACE1 | MGSENSALKSYTLREPPFTLPSGLAVYPAV (SEQ ID NO: 104) | 1 |
| Q9H6Q3 | SLA2 C20orf156 SLAP2 | MGSLPSRRKSLPSPSLSSSVQGQGPVTMEA (SEQ ID NO: 105) | 1 |
| O75716 | STK16 MPSK1 PKL12 TSF1 | MGHALCVCSRGTVIIDNKRYLFIQKLGEGG (SEQ ID NO: 106) | 1 |
| Q99487 | PAFAH2 | MGVNQSVGFPPVTGPHLVGCGDVMEGQNLQ (SEQ ID NO: 107) | 0 |
| P42684 | ABL2 ABLL ARG | MGQQVGRVGEAPGLQQPQPRGIRGSSAARP (SEQ ID NO: 108) | 0 |
| O43687 | AKAP7 AKAP15 AKAP18 | MGQLCCFPFSRDEGKISELESSSSAVLQRY (SEQ ID NO: 109) | 0 |
| Q9P2G1 | ANKIB1 KIAA1386 | MGNTTTKFRKALINGDENLACQIYENNPQL (SEQ ID NO: 110) | 0 |
| P61204 | ARF3 | MGNIFGNLLKSLIGKKEMRILMVGLDAAGK (SEQ ID NO: 111) | 0 |
| Q969Q4 | ARL11 ARLTS1 | MGSVNSRGHKAEAQVVMMGLDSAGKTTLLY (SEQ ID NO: 112) | 0 |
| Q8N4G2 | ARL14 ARF7 | MGSLGSKNPQTKQAQVLLLGLDSAGKSTLL (SEQ ID NO: 113) | 0 |
| Q8IVW1 | ARL17A ARL17P1; ARL17B | MGNIFEKLFKSLLGKKKMRILILSLDTAG (SEQ ID NO: 114) | 0 |
| | ARF1P2 ARL17A PRO2667 | | |
| P49703 | ARL4D ARF4L | MGNHLTEMAPTASSFLPHFQALHVVVIGLD (SEQ ID NO: 115) | 0 |
| Q9Y689 | ARL5A ARFLP5 ARL5 | MGILFTRIWRLFNHQEHKVIIVGLDNAGKT (SEQ ID NO: 116) | 0 |
| Q96KC2 | ARL5B ARL8 | MGLIFAKLWSLFCNQEHKVIIVGLDNAGKT (SEQ ID NO: 117) | 0 |
| A6NH57 | ARL5C ARL12 | MGQLIAKLMSIFGNQEHTVIIVGLDNEGKT (SEQ ID NO: 118) | 0 |
| Q8WXS3 | BAALC | MGCGGSRADAIEPRYYESWTRETESTWLTY (SEQ ID NO: 119) | 0 |
| P51451 | BLK | MGLVSSKKPDKEKPIKEKDKGQWSPLKVSA (SEQ ID NO: 120) | 0 |
| Q969J3 | BORCS5 LOH12CR1 | MGSEQSSEAESRPNDLNSSVTPSPAKHRAK (SEQ ID NO: 121) | 0 |
| Q9UPA5 | BSN KIAA0434 ZNF231 | MGNEVSLEGGAGDGPLPPGGAGPGPGPGPG (SEQ ID NO: 122) | 0 |
| Q9P203 | BTBD7 KIAA1525 | MGANASNYPHSCSPRVGGNSQAQQTFIGTS (SEQ ID NO: 123) | 0 |
| A6NGG8 | C2orf71 | MGCTPSHSDLVNSVAKSGIQFLKKPKAIRP (SEQ ID NO: 124) | 0 |
| Q9NZU7 | CABP1 | MGGGDGAAFKRPGDGARLQRVLGLGSRREP (SEQ ID NO: 125) | 0 |
| Q9NPB3 | CABP2 | MGNCAKRPWRRGPKDPLQWLGSPPRGSCPS (SEQ ID NO: 126) | 0 |
| A6NI79 | CCDC69 | MGCRHSRLSSCKPPKKKRQEPEPEQPPRPE (SEQ ID NO: 127) | 0 |
| Q15078 | CDK5R1 CDK5R NCK5A | MGTVLSLSPSYRKATLFEDGAATVGHYTAV (SEQ ID NO: 128) | 0 |
| Q13319 | CDK5R2 NCK5A1 | MGTVLSLSPASSAKGRRPGGLPEEKKKAPP (SEQ ID NO: 129) | 0 |
| O43745 | CHP2 HCA520 | MGSRSSHAAVIPDGDSIRRETGFSQASLLR (SEQ ID NO: 130) | 0 |
| Q717R9 | CYS1 | MGSGSSRSSRTLRRRRSPESLPAGPGAAAL (SEQ ID NO: 131) | 0 |
| Q6QHC5 | DEGS2 C14orf66 | MGNSASRSDFEWVYTDQPHTQRRKEILAKY (SEQ ID NO: 132) | 0 |
| Q9NRW4 | DUSP22 JSP1 LMWDSP2 | MGNGMNKILPGLYIGNFKDARDAEQLSKNK (SEQ ID NO: 133) | 0 |
| | MKPX | | |
| Q7RTS9 | DYM | MGSNSSRIGDLPKNEYLKKLSGTESISEND (SEQ ID NO: 134) | 0 |
| P16452 | EPB42 E42P | MGQALGIKSCDFQAARNNEEHHTKALSSRR (SEQ ID NO: 135) | 0 |
| P87889 | ERVK-10 | MGQTKSKIKSKYASYLSFIKILLKRGGVKV (SEQ ID NO: 136) | 0 |
| P62683 | ERVK-21 | MGQTKSKIKSKYASYLSFIKILLKRGGVKV (SEQ ID NO: 137) | 0 |
| P63145 | ERVK-24 | MGQTKSKIKSKYASYLSFIKILLKRGGVKV (SEQ ID NO: 138) | 0 |
| Q9HDB9 | ERVK-5 ERVK5 | MGQTKSKTKSKYASYLSFIKILLKRGGVRV (SEQ ID NO: 139) | 0 |
| Q7LDI9 | ERVK-6 ERVK6 | MGQTKSKIKSKYASYLSFIKILLKRGGVKV (SEQ ID NO: 140) | 0 |
| P62685 | ERVK-8 | MGQTKSKIKSKYASYLSFIKILLKRGGVKV (SEQ ID NO: 141) | 0 |
| P63126 | ERVK-9 | MGQTKSKIKSKYASYLSFIKILLKRGGVKV (SEQ ID NO: 142) | 0 |
| P63128 | ERVK-9 | MGQTKSKIKSKYASYLSFIKILLKRGGVKV (SEQ ID NO: 143) | 0 |
| P09769 | FGR SRC2 | MGCVFCKKLEPVATAKEDAGLEGDFRSYGA (SEQ ID NO: 144) | 0 |
| O95466 | FMNL1 C17orf1 C17orf1B | MGNAAGSAEQPAGPAAPPPKQPAPPKQPMP (SEQ ID NO: 145) | 0 |
| | FMNL FRL1 | | |
| O43559 | FRS3 | MGSCCSCLNRDSVPDNHPTKFKVTNVDDEG (SEQ ID NO: 146) | 0 |
| P11488 | GNAT1 GNATR | MGCTLSAEDKAAVERSKMIDRNLREDGEKA (SEQ ID NO: 147) | 0 |
| Q9BQQ3 | GORASP1 GOLPH5 GRASP65 | MGLGVSAEQPAGGAEGFHLHGVQENSPAQQ (SEQ ID NO: 148) | 0 |
| P43080 | GUCA1A C6orf131 GCAP | MGNVMEGKSVEELSSTECHQWYKKFMTECP (SEQ ID NO: 149) | 0 |
| | GCAP1 GUCA1 | | |
| Q9UMX6 | GUCA1B GCAP2 | MGQEFSWEEAEAAGEIDVAELQEWYKKFVM (SEQ ID NO: 150) | 0 |
| O95843 | GUCA1C GCAP3 | MGNGKSIAGDQKAVPTQETHVWYRTFMMEY (SEQ ID NO: 151) | 0 |
| P53701 | HCCS CCHL | MGLSPSAPAVAVQASNASASPPSGCPMHEG (SEQ ID NO: 152) | 0 |
| P62684 | HERVK_113 | MGQTKSKIKSKYASYLSFIKILLKRGGVKV (SEQ ID NO: 153) | 0 |
| Q8TB92 | HMGCLL1 | MGNVPSAVKHCLSYQQLLREHLWIGDSVAG (SEQ ID NO: 154) | 0 |
| P84074 | HPCA BDR2 | MGKQNSKLRPEMLQDLRENTEFSELELQEW (SEQ ID NO: 155) | 0 |
| Q9UM19 | HPCAL4 | MGKTNSKLAPEVLEDLVQNTEFSEQELKQW (SEQ ID NO: 156) | 0 |
| P63252 | KCNJ2 IRK1 | MGSVRTNRYSIVSSEEDGMKLATMAVANGF (SEQ ID NO: 157) | 0 |
| Q6VT66 | MARC1 MOSC1 | MGAAGSSALARFVLLAQSRPGWLGVAALGL (SEQ ID NO: 158) | 0 |
| P61601 | NCALD | MGKQNSKLRPEVMQDLLESTDFTEHEIQEW (SEQ ID NO: 159) | 0 |
| O76050 | NEURL1 NEURL NEURL1A | MGNNFSSIPSLPRGNPSRAPRGHPQNLKDS (SEQ ID NO: 160) | 0 |
| | RNF67 | | |
| Q969F2 | NKD2 | MGKLQSKHAAAARKRRESPEGDSFVASAYA (SEQ ID NO: 161) | 0 |
| P29474 | NOS3 | MGNLKSVAQEPGPPCGLGLGLGLGLCGKQG (SEQ ID NO: 162) | 0 |
| Q7Z494 | NPHP3 KIAA2000 | MGTASSLVSPAGGEVIEDTYGAGGGEACEI (SEQ ID NO: 163) | 0 |
| Q6X4W1 | NSMF NELF | LRSEAMSSVAAKVRAARAFG (SEQ ID NO: 164) | 0 |
| Q96MG8 | PCMTD1 | MGGAVSAGEDNDDLIDNLKEAQYIRTERVE (SEQ ID NO: 165) | 0 |
| Q9NV79 | PCMTD2 C20orf36 | MGGAVSAGEDNDELIDNLKEAQYIRTELVE (SEQ ID NO: 166) | 0 |
| O00408 | PDE2A | MGQACGHSILCRSQQYPAARPAEPRGQQVF (SEQ ID NO: 167) | 0 |
| Q9UPV7 | PHF24 KIAA1045 | MGVLMSKRQTVEQVQKVSLAVSAFKDGLRD (SEQ ID NO: 168) | 0 |
| Q494U1 | PLEKHN1 | MGNSHCVPQAPRRLRASFSRKPSLKGNRED (SEQ ID NO: 169) | 0 |
| P35813 | PPM1A PPPM1A | MGAFLDKPKMEKHNAQGQGNGLRYGLSSMQ (SEQ ID NO: 170) | 0 |
| Q96LZ3 | PPP3R2 CBLP PPP3RL | MGNEASYPAEMCSHFDNDEIKRLGRRFKKL (SEQ ID NO: 171) | 0 |
| Q9Y478 | PRKAB1 AMPK | MGNTSSERAALERHGGHKTPRRDSSGGTKD (SEQ ID NO: 172) | 0 |
| P22612 | PRKACG | MGNAPAKKDTEQEESVNEFLAKARGDFLYR (SEQ ID NO: 173) | 0 |
| Q13237 | PRKG2 PRKGR2 | MGNGSVKPKHSKHPDGHSGNLTTDALRNKV (SEQ ID NO: 174) | 0 |
| Q9NR22 | PRMT8 HRMT1L3 HRMT1L4 | MGMKHSSRCLLLRRKMAENAAESTEVNSPP (SEQ ID NO: 175) | 0 |
| P11801 | PSKH1 | MGCGTSKVLPEPPKDVQLDLVKKVEPFSGT (SEQ ID NO: 176) | 0 |
| Q13702 | RAPSN RNF205 | MGQDQTKQQIEKGLQLYQSNQTEKALQVWT (SEQ ID NO: 177) | 0 |
| P35243 | RCVRN RCV1 | MGNSKSGALSKEILEELQLNTKFSEEELCS (SEQ ID NO: 178) | 0 |
| Q96EQ8 | RNF125 | MGSVLSTDSGKSAPASATARALERRRDPEL (SEQ ID NO: 179) | 0 |
| Q8WVD5 | RNF141 ZNF230 | MGQQISDQTQLVINKLPEKVAKHVTLVRES (SEQ ID NO: 180) | 0 |
| Q96PX1 | RNF157 KIAA1917 | MGALTSRQHAGVEEVDIPSNSVYRYPPKSG (SEQ ID NO: 181) | 0 |
| Q13239 | SLA SLAP SLAP1 | MGNSMKSTPAPAERPLPNPEGLDSDFLAVL (SEQ ID NO: 182) | 0 |
| Q8WU08 | STK32A YANK1 | MGANTSRKPPVFDENEDVNFDHFEILRAIG (SEQ ID NO: 183) | 0 |
| H3BQB6 | STMND1 | MGCGPSQPAEDRRRVRAPKKGWKEEFKADV (SEQ ID NO: 184) | 0 |
| Q13009 | TIAM1 | MGNAESQHVEHEFYGEKHASLGRKHTSRSL (SEQ ID NO: 185) | 0 |
| Q81VF5 | TIAM2 KIAA2016 STEF | MGNSDSQYTLQGSKNHSNTITGAKQIPCSL (SEQ ID NO: 186) | 0 |
| Q86XR7 | TICAM2 TIRAP3 TIRP TRAM | MGIGKSKINSCPLSLSWGKRHSVDTSPGYH (SEQ ID NO: 187) | 0 |
| Q6P9B6 | TLDC1 KIAA1609 | MGNSRSRVGRSFCSQFLPEEQAEIDQLFDA (SEQ ID NO: 188) | 0 |
| Q9BVX2 | TMEM106C EMOC | MGSQHSAAARPSSCRRKQEDDRDGLLAERE (SEQ ID NO: 189) | 0 |
| P98073 | TMPRSS15 ENTK PRSS7 | MGSKRGISSRHHSLSSYEIMFAALFAILVV (SEQ ID NO: 190) | 0 |
| Q8ND25 | ZNRF1 NIN283 | MGGKQSTAARSRGPFPGVSTDDSAVPPPGG (SEQ ID NO: 191) | 0 |

TABLE 2
The number of the detected proteins and potentially myristoylated proteins in
Extracellular vesicles in 60 cancer cell lines

| | | Number of | Number of detected | Appearance frequency |
|---|---|---|---|---|
| | | detected proteins | potentially myristoylated | of myristoylated |
| Organs | Cell Lines | in exosomes | proteins in exosomes | protein in exosomes |
| Leukemia | SR | 1772 | 28 | 1.58 |
| Kidney | TK-10 | 1880 | 31 | 1.65 |
| Leukemia | RPMI-8226 | 1694 | 29 | 1.71 |
| Lung | HOP-62 | 1740 | 30 | 1.72 |
| Lung | NCI-H322M | 1208 | 21 | 1.74 |
| Leukemia | K562 | 2155 | 38 | 1.76 |
| Kidney | A498 | 2536 | 45 | 1.77 |
| Melanoma | LOX IMVI | 2382 | 43 | 1.81 |
| Kidney | ACHN | 1486 | 27 | 1.82 |
| Kidney | UO-31 | 1427 | 26 | 1.82 |
| Breast | MCF7 | 2299 | 42 | 1.83 |
| Lung | HOP-92 | 1525 | 28 | 1.84 |
| Colon | HT29 | 2059 | 38 | 1.85 |
| Ovary | OVCAR-3 | 2245 | 42 | 1.87 |
| Ovary | OVCAR-4 | 2717 | 51 | 1.88 |
| Leukemia | MOLT-4 | 2020 | 38 | 1.88 |
| Lung | EKVX | 1136 | 22 | 1.94 |
| Ovary | IGROV1 | 1699 | 33 | 1.94 |
| Breast | T-47D | 2092 | 41 | 1.96 |
| Leukemia | HL-60 | 1678 | 33 | 1.97 |
| Breast | BT549 | 2269 | 45 | 1.98 |
| Lung | NCI-H522 | 1608 | 32 | 1.99 |
| Melanoma | SK-MEL-5 | 2225 | 45 | 2.02 |
| Melanoma | UACC-62 | 1728 | 35 | 2.03 |
| Breast | MDA-MB-468 | 2377 | 49 | 2.06 |
| Colon | KM12 | 2423 | 50 | 2.06 |
| Colon | Colo205 | 2545 | 53 | 2.08 |
| Leukemia | CCRF-CEM | 2331 | 49 | 2.10 |
| Kidney | RXF 393 | 1830 | 39 | 2.13 |
| Lung | A549 | 1868 | 40 | 2.14 |
| Melanoma | SK-MEL-2 | 2262 | 49 | 2.17 |
| Ovary | SK-OV-3 | 1569 | 34 | 2.17 |
| Colon | HCT-15 | 2476 | 54 | 2.18 |
| Kidney | 786-O | 1442 | 32 | 2.22 |
| Lung | NCI-H23 | 1663 | 37 | 2.22 |
| Colon | HCT-116 | 2510 | 56 | 2.23 |
| Colon | SW620 | 2691 | 61 | 2.27 |
| Melanoma | M14 | 1409 | 32 | 2.27 |
| Lung | NCL-H226 | 1755 | 40 | 2.28 |
| Ovary | OVCAR-5 | 2000 | 46 | 2.30 |
| Melanoma | MALME-3M | 2074 | 48 | 2.31 |
| Lung | NCI-H460 | 1336 | 31 | 2.32 |
| Kidney | CAKI | 1401 | 33 | 2.36 |
| Breast | MDA-MB-231 | 2237 | 53 | 2.37 |
| CNS | SF295 | 2041 | 49 | 2.40 |
| Melanoma | SK-MEL-28 | 1817 | 44 | 2.42 |
| Colon | HCC 2998 | 1841 | 45 | 2.44 |
| CNS | U251 | 1862 | 46 | 2.47 |
| Melanoma | UACC-257 | 1940 | 48 | 2.47 |
| CNS | SNB-19 | 1857 | 46 | 2.48 |
| Ovary | NCI-ADR-RES | 2341 | 58 | 2.48 |
| CNS | SF539 | 1761 | 44 | 2.50 |
| Prostate | PC-3 | 1558 | 39 | 2.50 |
| Prostate | DU145 | 1274 | 32 | 2.51 |
| CNS | SNB-75 | 1909 | 48 | 2.51 |
| CNS | SF268 | 1819 | 46 | 2.53 |
| Kidney | SN12C | 1716 | 44 | 2.56 |
| Ovary | OVCAR-8 | 2005 | 53 | 2.64 |
| Melanoma | MDA-MB-435 | 1680 | 45 | 2.68 |
| Breast | HS 578T | 1228 | 34 | 2.77 |

TABLE 3
The potential myristoylated proteins detected in extracellular vesicles of breast milk.

| Protein | | The peptide sequence in the N-terminus of in |
|---|---|---|
| ID | Gene Name | potential myristoylated proteins |
| P18085 | ARF4 ARF2 | MGLTISSLFSRLFGKKQMRILMVGLDAAGK (SEQ ID NO: 192) |
| P62330 | ARF6 | MGKVLSKIFGNKEMWILMLGLDAAGKTTIL (SEQ ID NO: 193) |
| P04899 | GNAI2 GNAI2B | MGCTVSAEDKAAAERSKMIDKNLREDGEKA (SEQ ID NO: 194) |
| P08754 | GNAI3 | MGCTLSAEDKAAVERSKMIDRNLREDGEKA (SEQ ID NO: 195) |
| Q96TA1 | FAM129B C9orf88 | MGDVLSTHLDDARRQHIAEKTGKILTEFLQ (SEQ ID NO: 196) |
| Q6IAA8 | LAMTOR1 C11orf59 PDRO PP7157 | MGCCYSSENEDSDQDREERKLLLDPSSPPT (SEQ ID NO: 197) |
| Q14254 | FLOT2 ESA1 M17S1 | MGNCHTVGPNEALVVSGGCCGSDYKQYVFG (SEQ ID NO: 198) |
| P84085 | ARF5 | MGLTVSALFSRIFGKKQMRILMVGLDAAGK (SEQ ID NO: 199) |
| P61313 | RPL15 EC45 TCBAP0781 | MGAYKYIQELWRKKQSDVMRFLLRVRCWQY (SEQ ID NO: 200) |
| P07947 | YES1 YES | MGCIKSKENKSPAIKYRPENTPEPVSTSVS (SEQ ID NO: 201) |
| Q9NUQ9 | FAM49B BM-009 | MGNLLKVLTCTDLEQGPNFFLDFENAQPTE (SEQ ID NO: 202) |
| Q9H4G4 | GLIPR2 C9orf19 GAPR1 | MGKSASKQFHNEVLKAHNEYRQKHGVPPLK (SEQ ID NO: 203) |
| P63096 | GNAI1 | MGCTLSAEDKAAVERSKMIDRNLREDGEKA (SEQ ID NO: 204) |
| P36405 | ARL3 ARFL3 | MGLLSILRKLKSAPDQEVRILLLGLDNAGK (SEQ ID NO: 205) |
| Q96FZ7 | CHMP6 VPS20 | MGNLFGRKKQSRVTEQDKAILQLKQQRDKL (SEQ ID NO: 206) |
| Q99653 | CHP1 CHP | MGSRASTLLRDEELEEIKKETGFSHSQITR (SEQ ID NO: 207) |
| Q8WWI5 | SLC44A1 CD92 CDW92 CTL1 | MGCCSSASSAAQSSKREWKPLEDRSCTDIP (SEQ ID NO: 208) |
| P07948 | LYN JTK8 | MGCIKSKGKDSLSDDGVDLKTQPVRNTERT (SEQ ID NO: 209) |
| P49006 | MARCKSL1 MLP MRP | MGSQSSKAPRGDVTAEEAAGASPAKANGQE (SEQ ID NO: 210) |
| O75695 | RP2 | MGCFFSKRRKADKESRPENEEERPKQYSWD (SEQ ID NO: 211) |
| P29966 | MARCKS MACS PRKCSL | MGAQFSKTAAKGEAAAERPGEAAVASSPSK (SEQ ID NO: 212) |
| Q8N9N7 | LRRC57 | MGNSALRAHVETAQKTGVFQLKDRGLTEFP (SEQ ID NO: 213) |
| P37235 | HPCAL1 BDR1 | MGKQNSKLRPEVLQDLRENTEFTDHELQEW (SEQ ID NO: 214) |
| Q9NRX5 | SERINC1 KIAA1253 TDE1L TDE2 | MGSVLGLCSMASWIPCLCGSAPCLLCRCCP (SEQ ID NO: 215) |
| | UNQ396/PRO732 | |
| P40616 | ARL1 | MGGFFSSIFSSLFGTREMRILILGLDGAGK (SEQ ID NO: 216) |
| P80723 | BASP1 NAP22 | MGGKLSKKKKGYNVNDEKAKEKDKKAEGAA (SEQ ID NO: 217) |
| Q96PY5 | FMNL2 FHOD2 KIAA1902 | MGNAGSMDSQQTDFRAHNVPLKLPMPEPGE (SEQ ID NO: 218) |
| Q9BZQ8 | FAM129A C1orf24 NIBAN GIG39 | MGGSASSQLDEGKCAYIRGKTEAAIKNFSP (SEQ ID NO: 219) |
| Q8NHG7 | SVIP | MGLCFPCPGESAPPTPDLEEKRAKLAEAAE (SEQ ID NO: 220) |
| Q9Y3E7 | CHMP3 CGI149 NEDF VPS24 CGI-149 | MGLFGKTQEKPPKELVNEWSLKIRKEMRVV (SEQ ID NO: 221) |
| Q99828 | CIB1 CIB KIP PRKDCIP | MGGSGSRLSKELLAEYQDLTFLTKQEILLA (SEQ ID NO: 222) |
| P17612 | PRKACA PKACA | MGNAAAAKKGSEQESVKEFLAKAKEDFLKK (SEQ ID NO: 223) |
| Q8ND76 | CCNY C10orf9 CBCP1 CFP1 | MGNTTSCCVSSSPKLRRNAHSRLESYRPDT (SEQ ID NO: 224) |
| O00461 | GOLIM4 GIMPC GOLPH4 GPP130 | MGNGMCSRKQKRIFQTLLLLTVVFGFLYGA (SEQ ID NO: 225) |
| Q8NHG8 | ZNRF2 RNF202 | MGAKQSGPAAANGRTRAYSGSDLPSSSSGG (SEQ ID NO: 226) |
| P40617 | ARL4A ARL4 | MGNGLSDQTSILSNLPSFQSFHIVILGLDC (SEQ ID NO: 227) |
| O60291 | MGRN1 KIAA0544 RNF156 | MGSILSRRIAGVEDIDIQANSAYRYPPKSG (SEQ ID NO: 228) |
| Q9P2G1 | ANKIB1 KIAA1386 | MGNTTTKFRKALINGDENLACQIYENNPQL (SEQ ID NO: 229) |
| P61204 | ARF3 | MGNIFGNLLKSLIGKKEMRILMVGLDAAGK (SEQ ID NO: 230) |
| P35813 | PPM1A PPPM1A | MGAFLDKPKMEKHNAQGQGNGLRYGLSSMQ (SEQ ID NO: 231) |
| Q9Y478 | PRKAB1 AMPK | MGNTSSERAALERHGGHKTPRRDSSGGTKD (SEQ ID NO: 232) |

TABLE 4
The potential myristoylated proteins detected in exosomes of human thymus

| Protein | | The peptide sequence in the N-terminus of in |
|---|---|---|
| ID | Gene Name | potential myristoylated proteins |
| Q02952 | AKAP12 AKAP250 | MGAGSSTEQRSPEQPPEGSSTPAEPEPSGG (SEQ ID NO: 233) |
| P84077 | ARF1 | MGNIFANLFKGLFGKKEMRILMVGLDAAGK (SEQ ID NO: 234) |
| P18085 | ARF4 ARF2 | MGLTISSLFSRLFGKKQMRILMVGLDAAGK (SEQ ID NO: 235) |
| P84085 | ARF5 | MGLTVSALFSRIFGKKQMRILMVGLDAAGK (SEQ ID NO: 236) |
| P62330 | ARF6 | MGKVLSKIFGNKEMWILMLGLDAAGKTTIL (SEQ ID NO: 237) |
| P40616 | ARL1 | MGGFFSSIFSSLFGTREMRILILGLDGAGK (SEQ ID NO: 238) |
| P36405 | ARL3 ARFL3 | MGLLSILRKLKSAPDQEVRILLLGLDNAGK (SEQ ID NO: 239) |
| P80723 | BASP1 NAP22 | MGGKLSKKKKGYNVNDEKAKEKDKKAEGAA (SEQ ID NO: 240) |
| Q96FZ7 | CHMP6 VPS20 | MGNLFGRKKQSRVTEQDKAILQLKQQRDKL (SEQ ID NO: 241) |
| P00387 | CYB5R3 DIA1 | MGAQLSTLGHMVLFPVWFLYSLLMKLFQRS (SEQ ID NO: 242) |
| Q7L014 | DDX46 KIAA0801 | MGRESRHYRKRSASRGRSGSRSRSRSPSDK (SEQ ID NO: 243) |
| Q9BZQ8 | FAM129A C1orf24 NIBAN GIG39 | MGGSASSQLDEGKCAYIRGKTEAAIKNFSP (SEQ ID NO: 244) |
| Q96TA1 | FAM129B C9orf88 | MGDVLSTHLDDARRQHIAEKTGKILTEFLQ (SEQ ID NO: 245) |
| Q9NUQ9 | FAM49B BM-009 | MGNLLKVLTCTDLEQGPNFFLDFENAQPTE (SEQ ID NO: 246) |
| Q14254 | FLOT2 ESA1 M17S1 | MGNCHTVGPNEALVVSGGCCGSDYKQYVFG (SEQ ID NO: 247) |
| Q96PY5 | FMNL2 FHOD2 KIAA1902 | MGNAGSMDSQQTDFRAHNVPLKLPMPEPGE (SEQ ID NO: 248) |
| P06241 | FYN | MGCVQCKDKEATKLTEERDGSLNQSSGYRY (SEQ ID NO: 249) |
| Q9H4G4 | GLIPR2 C9orf19 GAPR1 | MGKSASKQFHNEVLKAHNEYRQKHGVPPLK (SEQ ID NO: 250) |
| P63096 | GNAI1 | MGCTLSAEDKAAVERSKMIDRNLREDGEKA (SEQ ID NO: 251) |
| P04899 | GNAI2 GNAI2B | MGCTVSAEDKAAAERSKMIDKNLREDGEKA (SEQ ID NO: 252) |
| P08754 | GNAI3 | MGCTLSAEDKAAVERSKMIDRNLREDGEKA (SEQ ID NO: 253) |
| Q9H8Y8 | GORASP2 GOLPH6 | MGSSQSVEIPGGGTEGYHVLRVQENSPGHR (SEQ ID NO: 254) |
| P08631 | HCK | MGGRSSCEDPGCPRDEERAPRMGCMKSKFL (SEQ ID NO: 255) |
| P37235 | HPCAL1 BDR1 | MGKQNSKLRPEVLQDLRENTEFTDHELQEW (SEQ ID NO: 256) |
| P06239 | LCK | MGCGCSSHPEDDWMENIDVCENCHYPIVPL (SEQ ID NO: 257) |
| Q8N9N7 | LRRC57 | MGNSALRAHVETAQKTGVFQLKDRGLTEFP (SEQ ID NO: 258) |
| P07948 | LYN JTK8 | MGCIKSKGKDSLSDDGVDLKTQPVRNTERT (SEQ ID NO: 259) |
| P29966 | MARCKS MACS PRKCSL | MGAQFSKTAAKGEAAAERPGEAAVASSPSK (SEQ ID NO: 260) |
| P49006 | MARCKSL1 MLP MRP | MGSQSSKAPRGDVTAEEAAGASPAKANGQE (SEQ ID NO: 261) |
| P08473 | MME EPN | MGKSESQMDITDINTPKPKKKQRWTPLEIS (SEQ ID NO: 262) |
| P29728 | OAS2 | MGNGESQLSSVPAQKLGWFIQEYLKPYEEC (SEQ ID NO: 263) |
| Q99570 | PIK3R4 VPS15 | MGNQLAGIAPSQILSVESYFSDIHDFEYDK (SEQ ID NO: 264) |
| P17612 | PRKACA PKACA | MGNAAAAKKGSEQESVKEFLAKAKEDFLKK (SEQ ID NO: 265) |
| P22694 | PRKACB | MGNAATAKKGSEVESVKEFLAKAKEDFLKK (SEQ ID NO: 266) |
| Q14699 | RFTN1 KIAA0084 MIG2 | MGCGLNKLEKRDEKRPGNIYSTLKRPQVET (SEQ ID NO: 267) |
| O75695 | RP2 | MGCFFSKRRKADKESRPENEEERPKQYSWD (SEQ ID NO: 268) |
| P61313 | RPL15 EC45 TCBAP0781 | MGAYKYIQELWRKKQSDVMRFLLRVRCWQY (SEQ ID NO: 269) |
| P62241 | RPS8 OK/SW-cl.83 | MGISRDNWHKRRKTGGKRKPYHKKRKYELG (SEQ ID NO: 270) |
| Q8WWI5 | SLC44A1 CD92 CDW92 CTL1 | MGCCSSASSAAQSSKREWKPLEDRSCTDIP (SEQ ID NO: 271) |
| P12931 | SRC SRC1 | MGSNKSKPKDASQRRRSLEPAENVHGAGGG (SEQ ID NO: 272) |
| P07947 | YES1 YES | MGCIKSKENKSPAIKYRPENTPEPVSTSVS (SEQ ID NO: 273) |
| O43149 | ZZEF1 KIAA0399 | MGNAPSHSSEDEAAAAGGEGWGPHQDWAAV (SEQ ID NO: 274) |
| P61204 | ARF3 | MGNIFGNLLKSLIGKKEMRILMVGLDAAGK (SEQ ID NO: 275) |
| O95466 | FMNL1 C17orf1 C17orf1B FMNL FRL1 | MGNAAGSAEQPAGPAAPPPKQPAPPKQPMP (SEQ ID NO: 276) |
| P11488 | GNAT1 GNATR | MGCTLSAEDKAAVERSKMIDRNLREDGEKA (SEQ ID NO: 277) |
| P61601 | NCALD | MGKQNSKLRPEVMQDLLESTDFTEHEIQEW (SEQ ID NO: 278) |
| O00408 | PDE2A | MGQACGHSILCRSQQYPAARPAEPRGQQVF (SEQ ID NO: 279) |
| Q9NR22 | PRMT8 HRMT1L3 HRMT1L4 | MGMKHSSRCLLLRRKMAENAAESTEVNSPP (SEQ ID NO: 280) |

TABLE 5
The potential myristoylated proteins detected in extracellular vesicles of human urine.

| Protein | | The peptide sequence in the N-terminus of in |
|---|---|---|
| ID | Gene Name | potential myristoylated proteins |
| Q9BRQ8 | AIFM2 AMID PRG3 | MGSQVSVESGALHVVIVGGGFGGIAAASQL (SEQ ID NO: 281) |
| Q02952 | AKAP12 AKAP250 | MGAGSSTEQRSPEQPPEGSSTPAEPEPSGG (SEQ ID NO: 282) |
| P27216 | ANXA13 ANX13 | MGNRHAKASSPQGFDVDRDAKKLNKACKGM (SEQ ID NO: 283) |
| P84077 | ARF1 | MGNIFANLFKGLFGKKEMRILMVGLDAAGK (SEQ ID NO: 284) |
| P18085 | ARF4 ARF2 | MGLTISSLFSRLFGKKQMRILMVGLDAAGK (SEQ ID NO: 285) |
| P84085 | ARF5 | MGLTVSALFSRIFGKKQMRILMVGLDAAGK (SEQ ID NO: 286) |
| P62330 | ARF6 | MGKVLSKIFGNKEMWILMLGLDAAGKTTIL (SEQ ID NO: 287) |
| P36405 | ARL3 ARFL3 | MGLLSILRKLKSAPDQEVRILLLGLDNAGK (SEQ ID NO: 288) |
| Q9H0F7 | ARL6 BBS3 | MGLLDRLSVLLGLKKKEVHVLCLGLDNSGK (SEQ ID NO: 289) |
| P80723 | BASP1 NAP22 | MGGKLSKKKKGYNVNDEKAKEKDKKAEGAA (SEQ ID NO: 290) |
| Q8ND76 | CCNY C10orf9 CBCP1 CFP1 | MGNTTSCCVSSSPKLRRNAHSRLESYRPDT (SEQ ID NO: 291) |
| Q9Y3E7 | CHMP3 CGI149 NEDF VPS24 CGI-149 | MGLFGKTQEKPPKELVNEWSLKIRKEMRVV (SEQ ID NO: 292) |
| Q96FZ7 | CHMP6 VPS20 | MGNLFGRKKQSRVTEQDKAILQLKQQRDKL (SEQ ID NO: 293) |
| Q99653 | CHP1 CHP | MGSRASTLLRDEELEEIKKETGFSHSQITR (SEQ ID NO: 294) |
| Q99828 | CIB1 CIB KIP PRKDCIP | MGGSGSRLSKELLAEYQDLTFLTKQEILLA (SEQ ID NO: 295) |
| P00387 | CYB5R3 DIA1 | MGAQLSTLGHMVLFPVWFLYSLLMKLFQRS (SEQ ID NO: 296) |
| Q9BZQ8 | FAM129A C1orf24 NIBAN GIG39 | MGGSASSQLDEGKCAYIRGKTEAAIKNFSP (SEQ ID NO: 297) |
| Q96TA1 | FAM129B C9orf88 | MGDVLSTHLDDARRQHIAEKTGKILTEFLQ (SEQ ID NO: 298) |
| Q9NUQ9 | FAM49B BM-009 | MGNLLKVLTCTDLEQGPNFFLDFENAQPTE (SEQ ID NO: 299) |
| Q14254 | FLOT2 ESA1 M17S1 | MGNCHTVGPNEALVVSGGCCGSDYKQYVFG (SEQ ID NO: 300) |
| P06241 | FYN | MGCVQCKDKEATKLTEERDGSLNQSSGYRY (SEQ ID NO: 301) |
| Q9H4G4 | GLIPR2 C9orf19 GAPR1 | MGKSASKQFHNEVLKAHNEYRQKHGVPPLK (SEQ ID NO: 302) |
| P63096 | GNAI1 | MGCTLSAEDKAAVERSKMIDRNLREDGEKA (SEQ ID NO: 303) |
| P04899 | GNAI2 GNAI2B | MGCTVSAEDKAAAERSKMIDKNLREDGEKA (SEQ ID NO: 304) |
| P08754 | GNAI3 | MGCTLSAEDKAAVERSKMIDRNLREDGEKA (SEQ ID NO: 305) |
| P09471 | GNAO1 | MGCTLSAEERAALERSKAIEKNLKEDGISA (SEQ ID NO: 306) |
| P19086 | GNAZ | MGCRQSSEEKEAARRSRRIDRHLRSESQRQ (SEQ ID NO: 307) |
| O00461 | GOLIM4 GIMPC GOLPH4 GPP130 | MGNGMCSRKQKRIFQTLLLLTVVFGFLYGA (SEQ ID NO: 308) |
| P08631 | HCK | MGGRSSCEDPGCPRDEERAPRMGCMKSKFL (SEQ ID NO: 309) |
| Q8IV36 | HID1 C17orf28 DM01 | MGSTDSKLNFRKAVIQLTTKTQPVEATDDA (SEQ ID NO: 310) |
| P37235 | HPCAL1 BDR1 | MGKQNSKLRPEVLQDLRENTEFTDHELQEW (SEQ ID NO: 311) |
| Q6IAA8 | LAMTOR1 C11orf59 PDRO PP7157 | MGCCYSSENEDSDQDREERKLLLDPSSPPT (SEQ ID NO: 312) |
| P06239 | LCK | MGCGCSSHPEDDWMENIDVCENCHYPIVPL (SEQ ID NO: 313) |
| Q8N9N7 | LRRC57 | MGNSALRAHVETAQKTGVFQLKDRGLTEFP (SEQ ID NO: 314) |
| P29966 | MARCKS MACS PRKCSL | MGAQFSKTAAKGEAAAERPGEAAVASSPSK (SEQ ID NO: 315) |
| P49006 | MARCKSL1 MLP MRP | MGSQSSKAPRGDVTAEEAAGASPAKANGQE (SEQ ID NO: 316) |
| O60291 | MGRN1 KIAA0544 RNF156 | MGSILSRRIAGVEDIDIQANSAYRYPPKSG (SEQ ID NO: 317) |
| P08473 | MME EPN | MGKSESQMDITDINTPKPKKKQRWTPLEIS (SEQ ID NO: 318) |
| O75688 | PPM1B PP2CB | MGAFLDKPKTEKHNAHGAGNGLRYGLSSMQ (SEQ ID NO: 319) |
| P17612 | PRKACA PKACA | MGNAAAAKKGSEQESVKEFLAKAKEDFLKK (SEQ ID NO: 320) |
| P22694 | PRKACB | MGNAATAKKGSEVESVKEFLAKAKEDFLKK (SEQ ID NO: 321) |
| Q14699 | RFTN1 KIAA0084 MIG2 | MGCGLNKLEKRDEKRPGNIYSTLKRPQVET (SEQ ID NO: 322) |
| O75695 | RP2 | MGCFFSKRRKADKESRPENEEERPKQYSWD (SEQ ID NO: 323) |
| P62241 | RPS8 OK/SW-cl.83 | MGISRDNWHKRRKTGGKRKPYHKKRKYELG (SEQ ID NO: 324) |
| Q9NRX5 | SERINC1 KIAA1253 TDE1L TDE2 | MGSVLGLCSMASWIPCLCGSAPCLLCRCCP (SEQ ID NO: 325) |
| | UNQ396/PRO732 | |
| Q8WWI5 | SLC44A1 CD92 CDW92 CTL1 | MGCCSSASSAAQSSKREWKPLEDRSCTDIP (SEQ ID NO: 326) |
| P12931 | SRC SRC1 | MGSNKSKPKDASQRRRSLEPAENVHGAGGG (SEQ ID NO: 327) |
| Q8NHG7 | SVIP | MGLCFPCPGESAPPTPDLEEKRAKLAEAAE (SEQ ID NO: 328) |
| P07947 | YES1 YES | MGCIKSKENKSPAIKYRPENTPEPVSTSVS (SEQ ID NO: 329) |
| Q9P2G1 | ANKIB1 KIAA1386 | MGNTTTKFRKALINGDENLACQIYENNPQL (SEQ ID NO: 330) |
| P61204 | ARF3 | MGNIFGNLLKSLIGKKEMRILMVGLDAAGK (SEQ ID NO: 331) |
| Q9P203 | BTBD7 KIAA1525 | MGANASNYPHSCSPRVGGNSQAQQTFIGTS (SEQ ID NO: 332) |
| Q717R9 | CYS1 | MGSGSSRSSRTLRRRRSPESLPAGPGAAAL (SEQ ID NO: 333) |
| Q7Z494 | NPHP3 KIAA2000 | MGTASSLVSPAGGEVIEDTYGAGGGEACEI (SEQ ID NO: 334) |
| P35813 | PPM1A PPPM1A | MGAFLDKPKMEKHNAQGQGNGLRYGLSSMQ (SEQ ID NO: 335) |
| Q9Y478 | PRKAB1 AMPK | MGNTSSERAALERHGGHKTPRRDSSGGTKD (SEQ ID NO: 336) |
| Q13237 | PRKG2 PRKGR2 | MGNGSVKPKHSKHPDGHSGNLTTDALRNKV (SEQ ID NO: 337) |
| P11801 | PSKH1 | MGCGTSKVLPEPPKDVQLDLVKKVEPFSGT (SEQ ID NO: 338) |
| Q6P9B6 | TLDC1 KIAA1609 | MGNSRSRVGRSFCSQFLPEEQAEIDQLFDA (SEQ ID NO: 339) |

Src Kinase is Detected and/or Enriched in EVs of Prostate Cancer Cells.

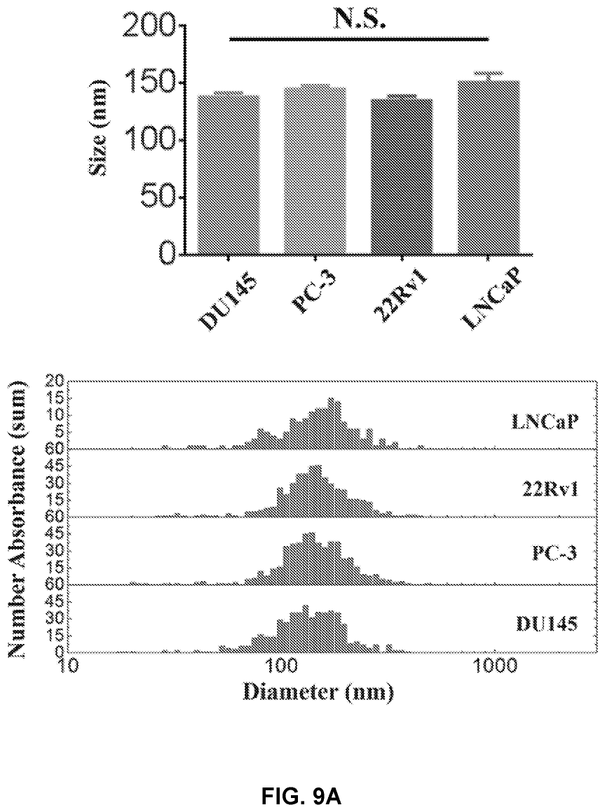

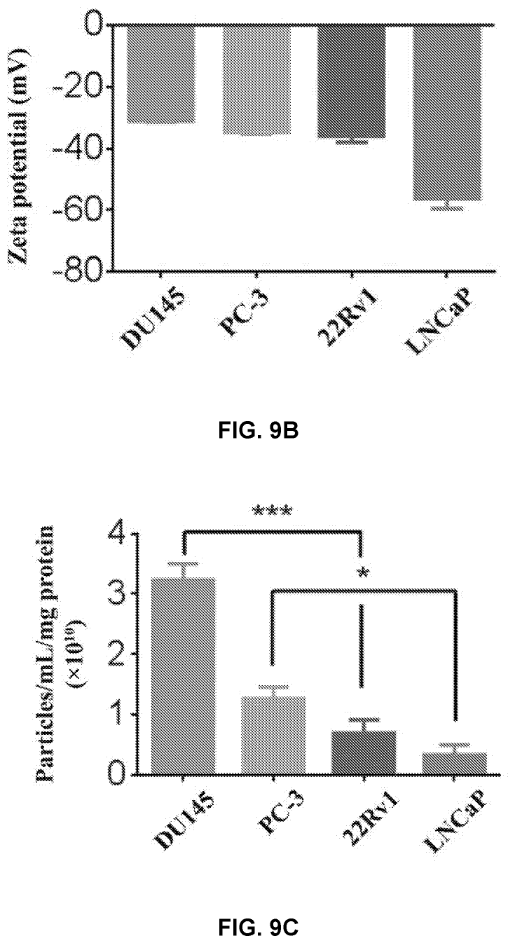

Src kinase has been well known to be myristoylated (Kim S, et al. Cancer Res. 2017 77:6950-62; Patwardhan P, et al. Mol Cell Biol. 2010 30:4094-107). To examine how myristoylation contributes to the encapsulation of a protein into EVs, we focused on Src kinase in EVs of four prostate cancer cell lines including PC3, DU145, LNCaP, and 22Rv1 cells. The average size of EVs derived from these cell lines was about 140 nm, and the size distribution showed no significant difference (FIG. 9A). The zeta potential of EVs ranged from −30 mV to −60 mV (FIG. 9B). Similar to CD9 and unlike androgen receptor or calnexin, Src kinase expression was detected in EVs from all tested cancer cell lines (FIG. 1C). While expression levels of Src kinase in EVs were equivalent to that in total cell lysate in 22Rv1 and LNCaP cells based on the same amount of protein loaded, Src kinase levels were 3 and 1.7-fold higher in EVs in comparison with total cell lysates in DU145 and PC3 cells, respectively (FIG. 1C). Correspondingly, the number of EVs derived from DU145 cells was significantly higher than that from other cells (FIG. 9C). An increase of the enrichment of Src kinase in EVs from PC3 and DU145 cells might be due to higher EVs biogenesis, which is reflected by an increased number of EVs in these cancer cells. Collectively, the data suggest that Src kinase, a myristoylated protein, is encapsulated into EVs, or enriched in EVs of cancer cells.

Myristoylation Mediates the Encapsulation of Src Kinase into EVs.

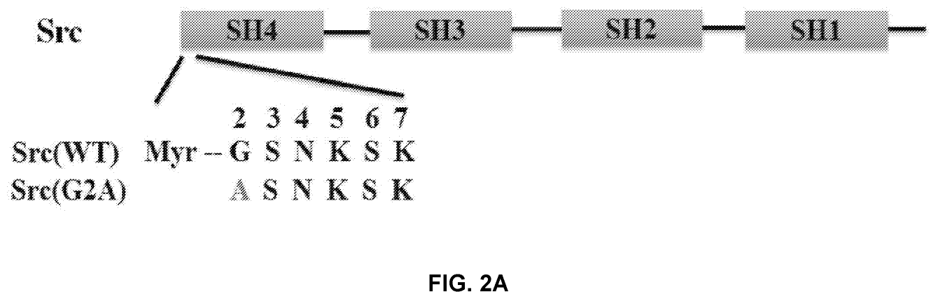

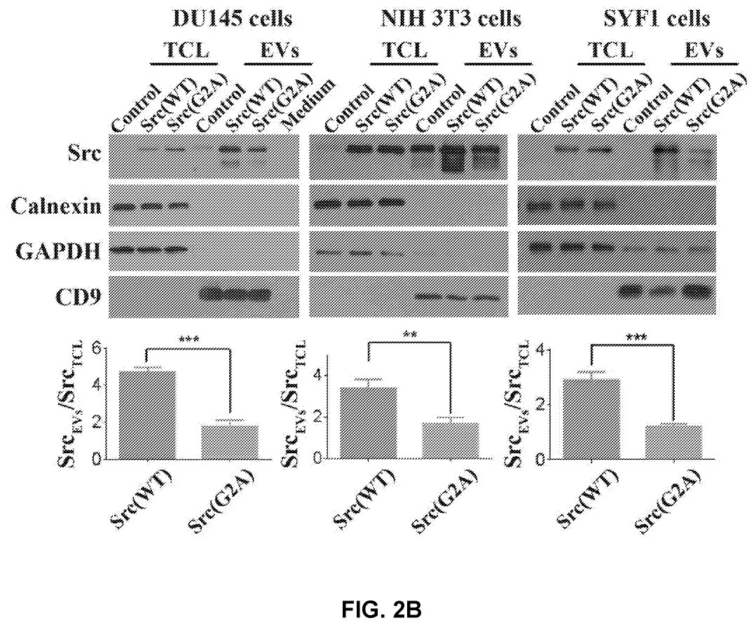

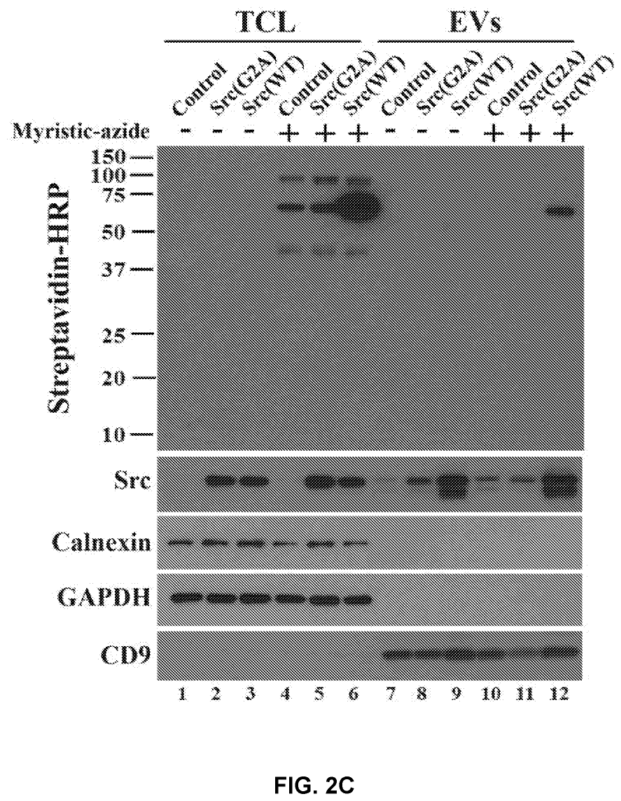

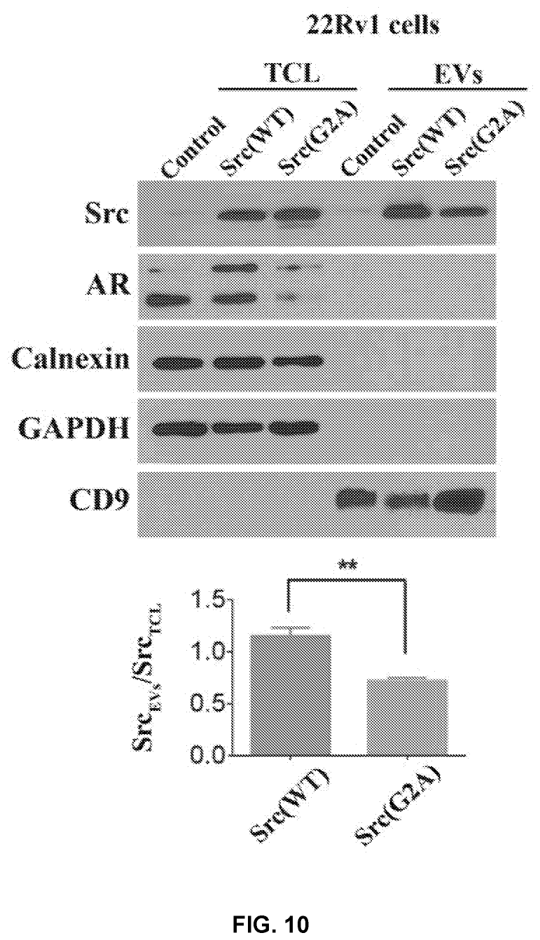

To examine the role of myristoylation in the encapsulation of Src kinase, four cell lines including DU145, NIH 3T3, SYF1, and 22Rv1 were transduced with wild type Src [Src(WT)] or Src(G2A), a mutant with loss of myristoylation by lentiviral infection (FIG. 2A). Levels of Src kinase were significantly reduced in EVs derived from all the tested cells expressing Src(G2A) in comparison with those expressing Src(WT) (FIGS. 2B and 10), suggesting that myristoylation plays an important role in mediating the encapsulation of Src kinase into EVs.

To further analyze if Src protein in EVs was myristoylated, DU145 cells expressing vector control, Src(WT), or Src(G2A) cells were cultured in medium containing myristic acid-azide (MA-azide, an analog of myristic acid). As expected, the endogenous Src levels in EVs were increased in comparison with that in total cell lysate (FIG. 2C, lane 1 and 4 versus lane 7 and 10, respectively). Src kinase levels were significantly elevated in EVs compared to those in total cell lysate in DU145 cells expressing ectopic levels of Src kinase (FIG. 2C, lane 3 versus lane 9; lane 6 versus lane 12), but not in cells expressing Src(G2A) mutant (lane 2 and 5 versus lane 8 and 11, respectively). As expected, the Src(G2A) mutant inhibits protein myristoylation (FIG. 2C, lane 5 vs 6, detected by streptavidin-HRP). In contrast, levels of myristoylated Src were significantly enriched in EVs in the DU145 cells expressing ectopic levels of Src kinase (FIG. 2C, lane 12 versus lane 11 or lane 10). Protein bands below 60 KD molecular weight were also detected, these proteins might be other members of Src family kinases detected by anti-Src antibody or non-myristoylated Src because the band was not observed in myristoylated proteins (FIG. 2C). The data indicate that Src kinase preferentially encapsulated into EVs is myristoylated.

An Increase of Src Kinase Activity Enhances its Encapsulation into EVs.

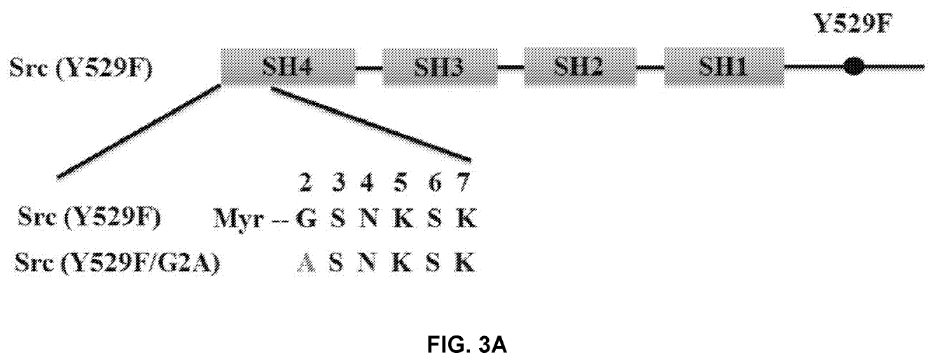

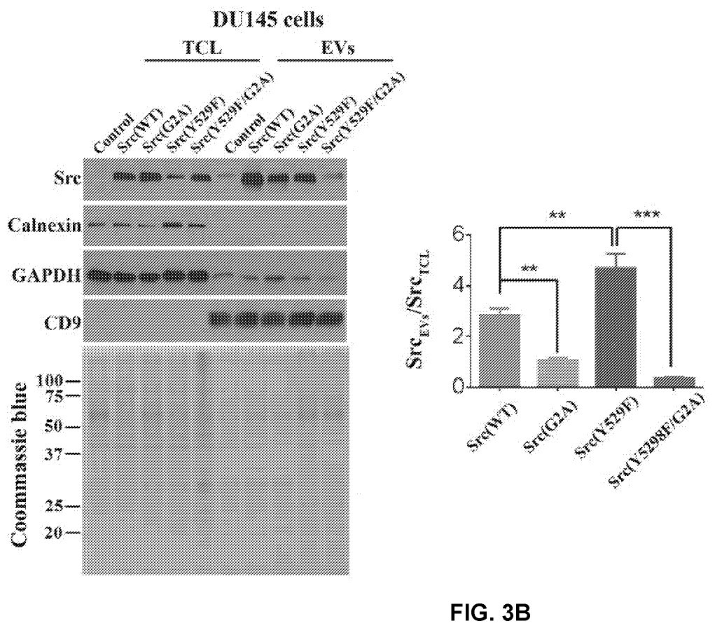

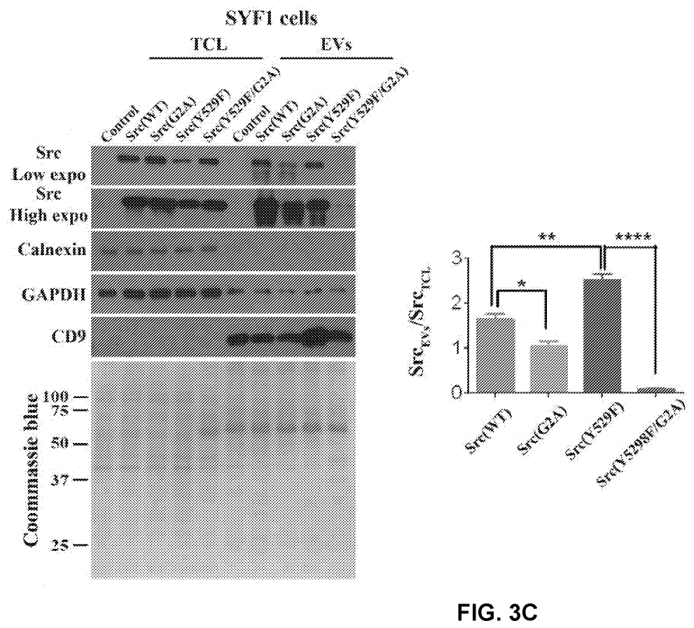

Src(Y529F) is a constitutively active Src kinase mutant (FIG. 3A). Similar to the enrichment of Src kinase in EVs [Src(WT) versus Src(G2A)], Src protein levels were significantly elevated in EVs from DU145 or SYF1 cells expressing Src(Y529F) in comparison with those expressing Src(Y529F/G2A) (FIGS. 3B-3C). Additionally, the ratio of Src kinase levels in EVs versus total cell lysate in DU145 or SYF1 cells expressing Src(Y529F) was elevated compared to that expressing Src(WT) (FIGS. 3B-3C). The data suggest that an increase of Src kinase activity enhances its encapsulation into EVs, however loss of myristoylation diminishes the preferential encapsulation of Src into EVs stimulated by the constitutive activity.

Palmitoylation Inhibits the Encapsulation of Proteins into EVs.

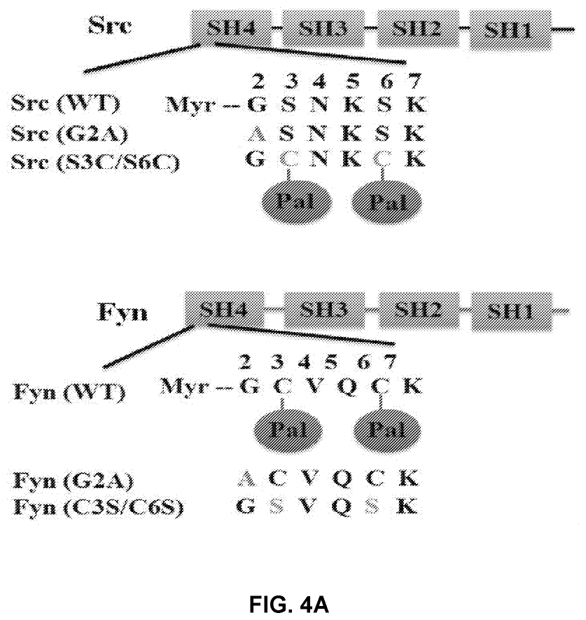

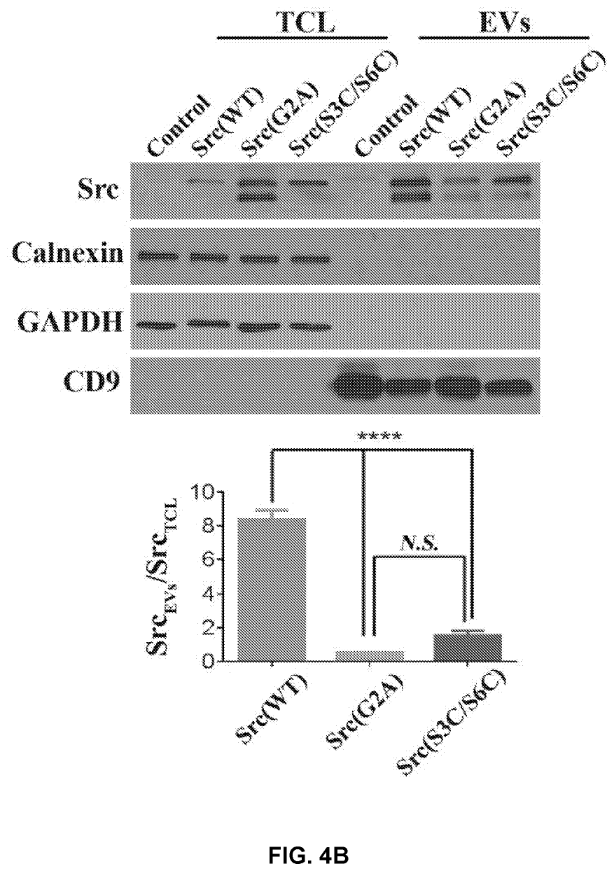

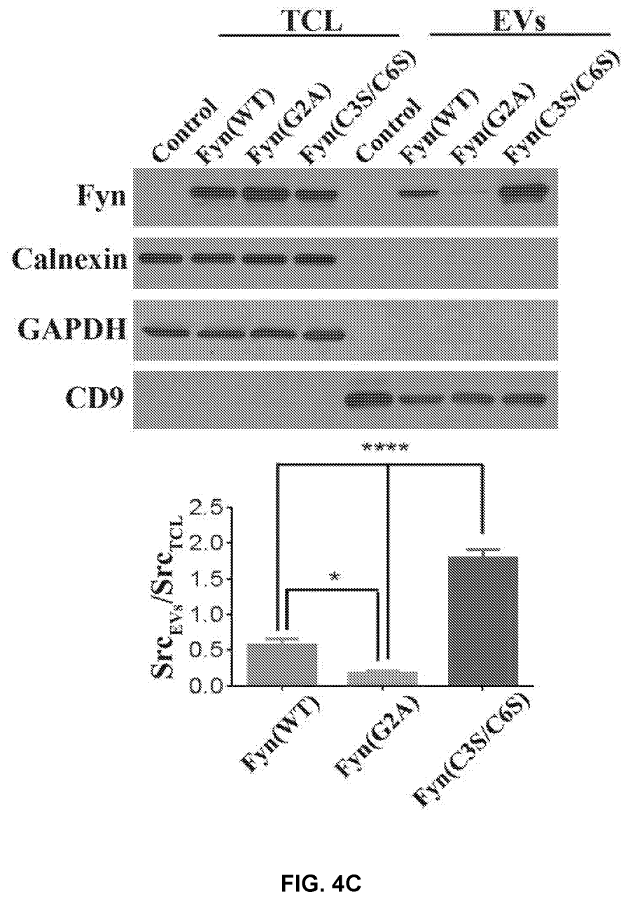

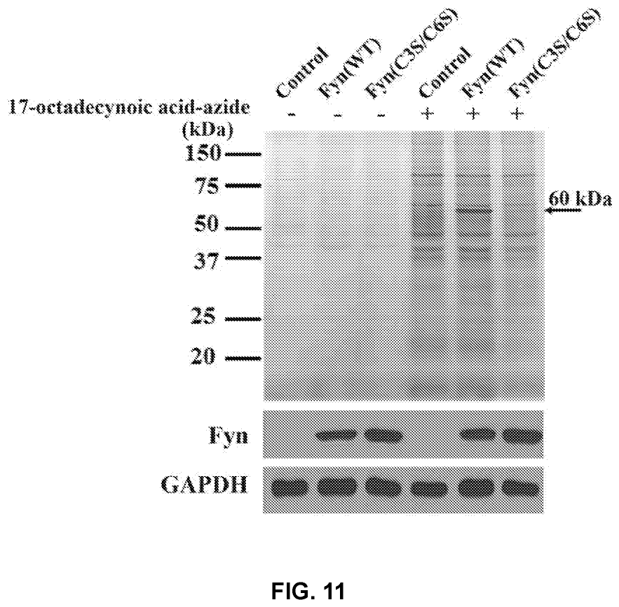

Some SFK members such as Fyn kinase are both myristoylated and palmitoylated at the N-terminus (Resh M D. Cell. 1994 76:411-3; Aicart-Ramos C, et al. 2011 1808:2981-94). A goal was set to study the role of palmitoylation in the regulation of protein encapsulation into EVs. Gain of palmitoylation sites in the Src(S3C/S6C) mutant, or loss of palmitoylation sites in the Fyn(C3S/C6S) mutant were previously created (FIG. 4A) (Cai H, et al. Proc Natl Acad Sci USA. 2011 108:6579-84). Over-expression of Fyn kinase and loss of palmitoylation were confirmed in SYF1 cells expressing control vector, wild type Fyn [Fyn(WT)], or Fyn(C3S/C6S) (FIG. 11). As expected, levels of Src kinase in EVs were elevated in comparison with that in total cell lysate in DU145 cells expressing ectopic Src(WT). However, levels of Src kinase in EVs from DU145 cells expressing Src(G2A) or Src(S3C/S6C) were significantly inhibited compared to that expressing Src(WT) (FIG. 4B). In contrast to cells expressing Src(WT), levels of Fyn kinase in EVs were decreased in comparison with that in total cell lysate from DU145 cells expressing Fyn(WT) (FIG. 4C). However, levels of Fyn kinase in EVs from cells expressing Fyn(C3S/C6S) were significantly increased in comparison with that expressing Fyn(WT). Additionally, levels of Fyn in EVs from cells expressing Fyn(G2A) were significantly inhibited compared to that expressing Fyn(WT) or Fyn(C3S/C6S). Collectively, the results indicate that opposite to myristoylation, palmitoylation inhibits the encapsulation of SFK members into EVs.

Myristoylation Mediates the Encapsulation of Src Kinase into Plasma EVs.

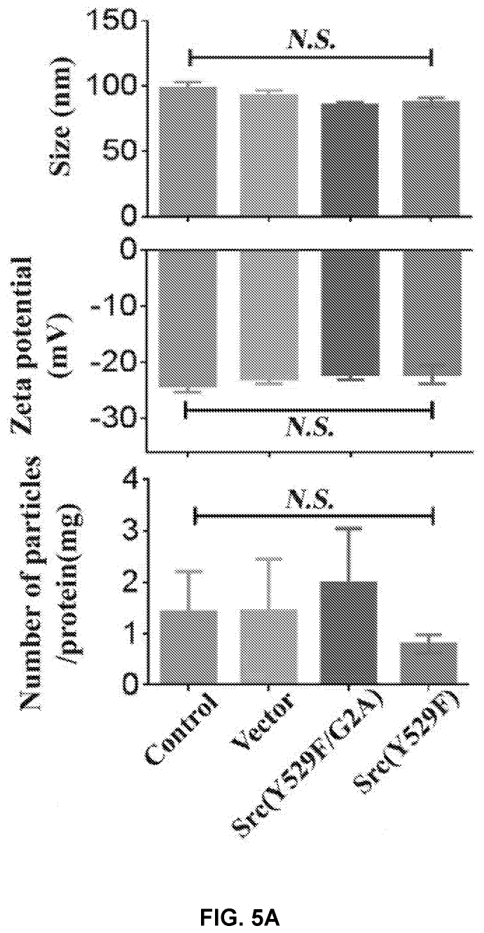

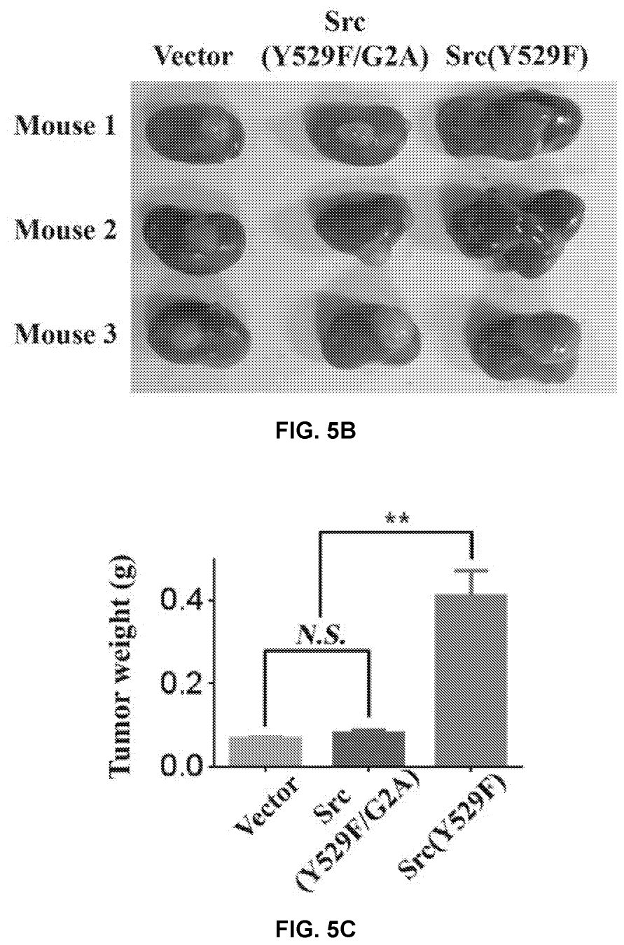

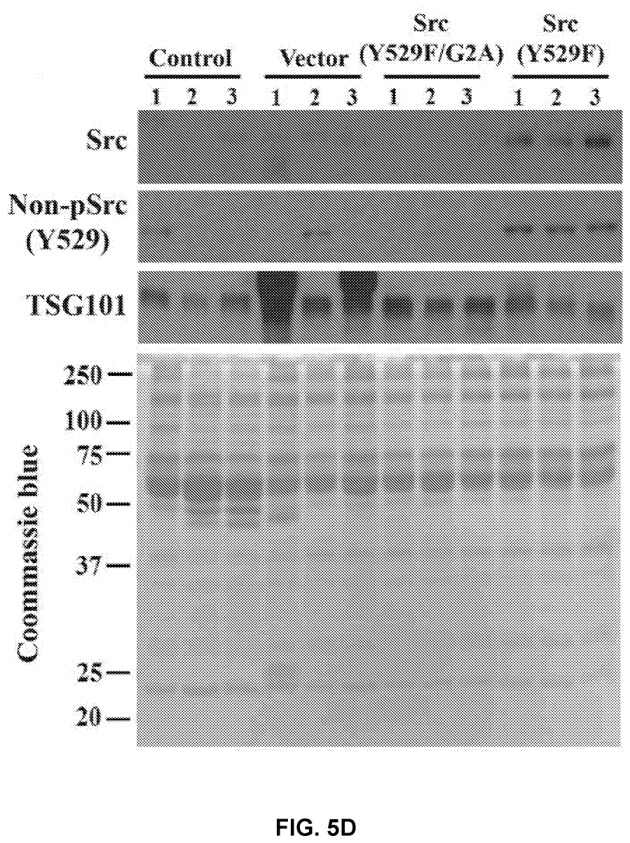

To further investigate if myristoylation mediates Src encapsulation into plasma EVs in vivo, DU145 cells or DU145 cells expressing vector control, Src(Y529F), or Src(Y529F/G2A) were implanted sub-renally into SCID mice. The isolated plasma EVs were characterized as mono-dispersed particles with the average size of −100 nm and zeta potential of −25 mV. This size and zeta potential were not significantly different among those isolated from xenograft-free mice, or mice carrying DU145 xenografts expressing control vector, Src(Y529F/G2A), or Src(Y529F) (FIG. 5A). As expected, since Src(Y529F) has higher oncogenic potential (Patwardhan P, et al. Mol Cell Biol. 2010 30:4094-107), the size and weight of xenografts expressing Src(Y529F) were significantly higher in comparison with those expressing vector control or Src(Y529F/G2A) (FIGS. 5B-5C). While expression levels of TSG101 (a marker of exosomal protein) were varied and not significantly different among the treatment groups, Src kinase levels in the plasma EVs from mice carrying xenograft tumors expressing Src(Y529F) were significantly elevated compared to those from mice without xenograft tumors (control), or xenograft tumors expressing control vector or Src(Y529F/G2A) (FIG. 5D). The results indicate that myristoylation is important to mediate Src encapsulation into plasma EVs in vivo.

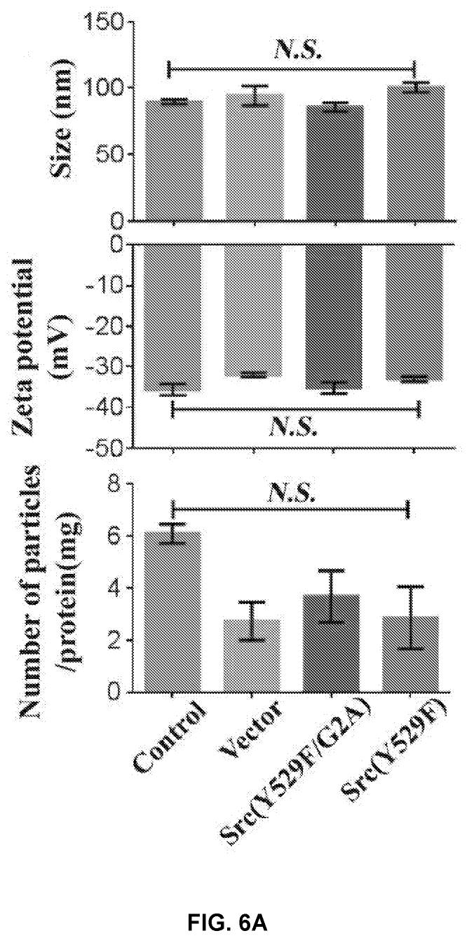

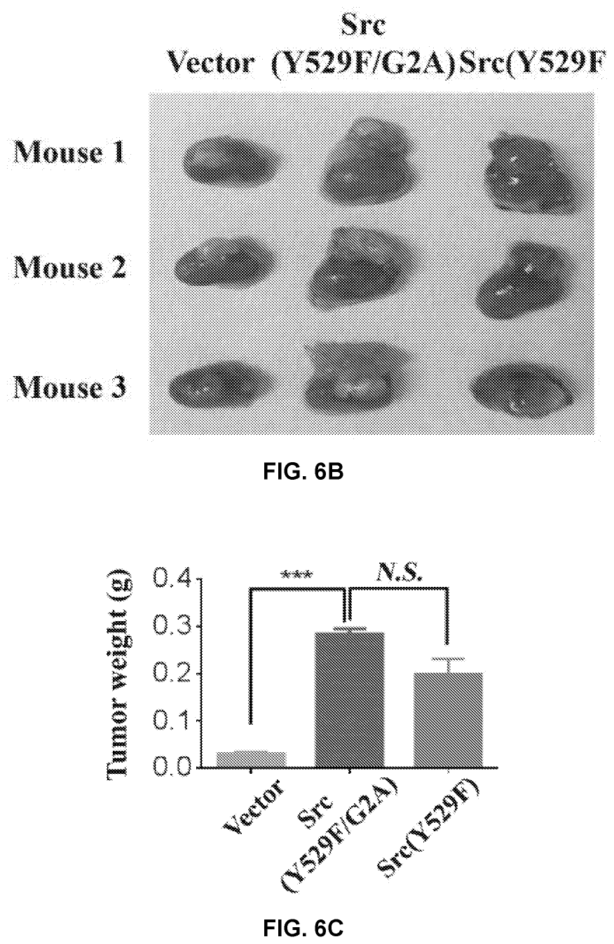

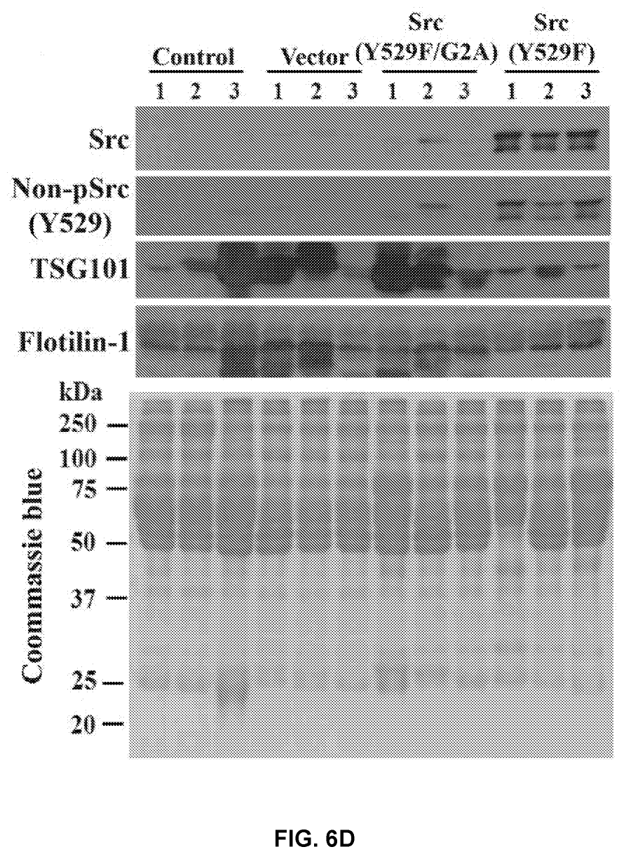

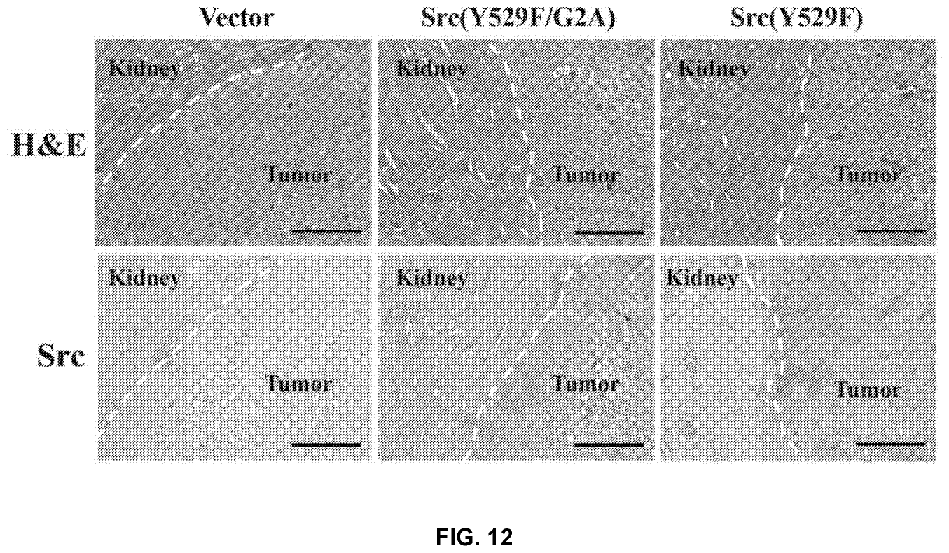

To exclude the possibility that higher Src levels in the plasma EVs were due to larger tumor size of Src(Y529F) induced xenograft tumors, ten times more DU145 cells or DU145 cells expressing Src(Y529F/G2A) were implanted relative to those expressing Src(Y529F). Similar to the previous experiment, the size and zeta potential were not significantly different among the plasma EVs in the different groups (FIG. 6A). Particularly, the weight of xenograft tumors showed no significant difference between the Src(Y529F) and Src(Y529F/G2A) groups (FIGS. 6B-6C). Expression levels of Src were confirmed by immunohistochemistry (FIG. 12). While expression levels of TSG101 and flotillin-1 (marker proteins in EVs) varied but showed no significant difference among experimental groups, expression levels of Src and non-phosphorylated Src(Y529) in the plasma EVs were significantly elevated in the Src(Y529F) group in comparison with Src(Y529F/G2A) or vector control groups (FIG. 6F). The results indicate that the detection of Src kinase in the plasma EVs was not due to the size of xenograft tumors, and myristoylation plays an essential role for the encapsulation of Src kinase in the plasma EVs. The data suggest that Src levels in plasma EVs may be a biomarker to identify Src-mediated xenograft tumors.

The encapsulation of Src kinase into EVs is mediated through the ESCRT pathway, not the lipid rafts pathway.

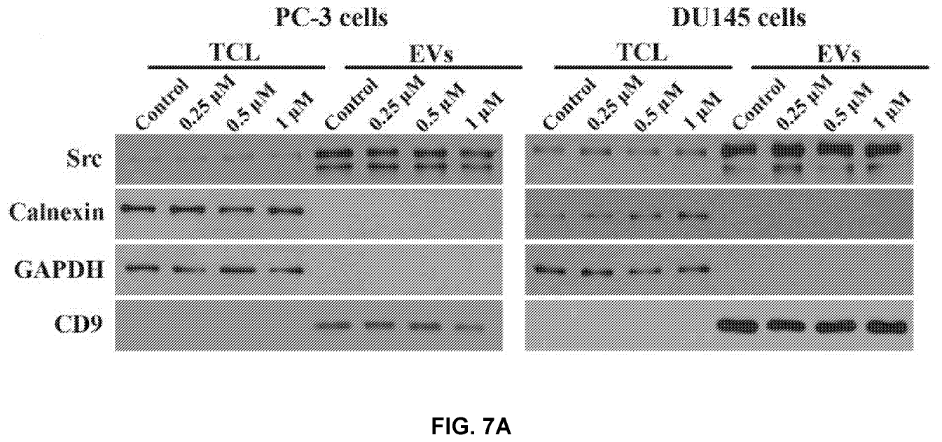

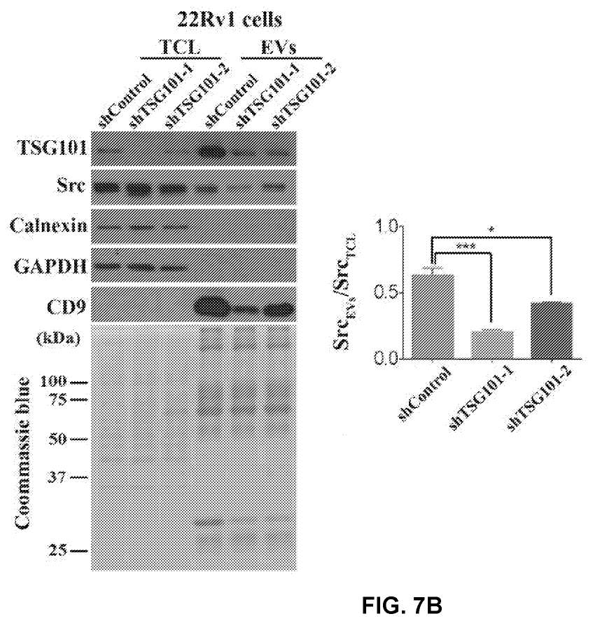

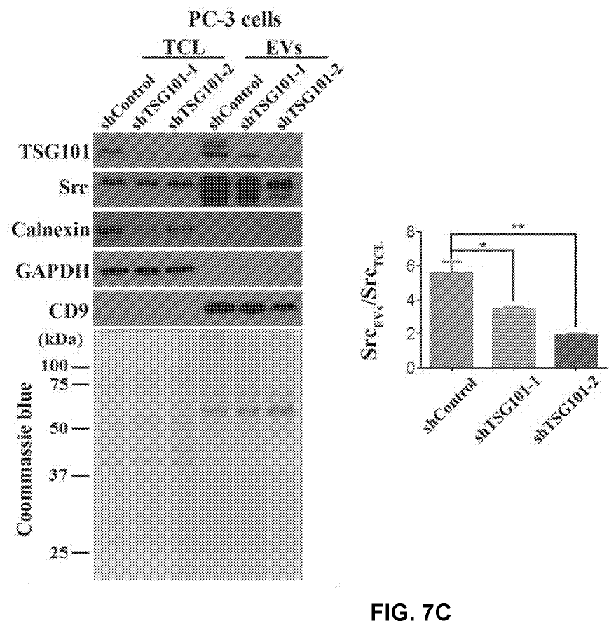

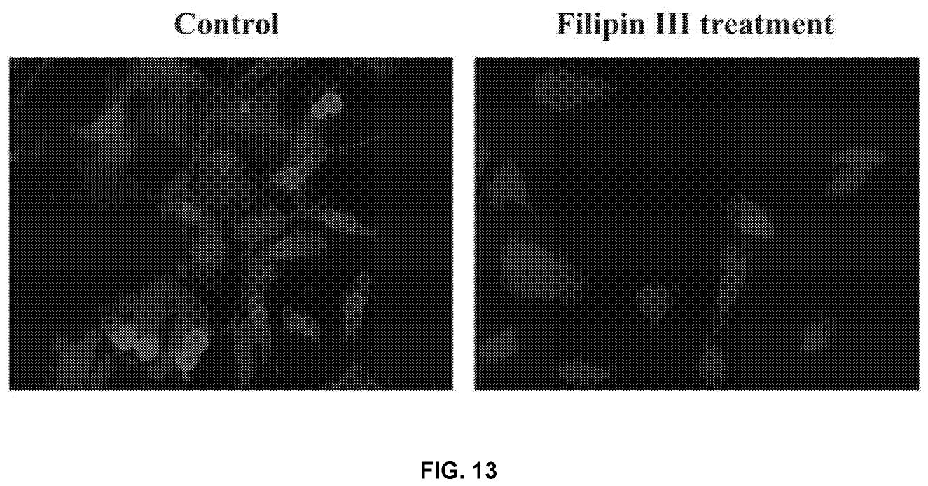

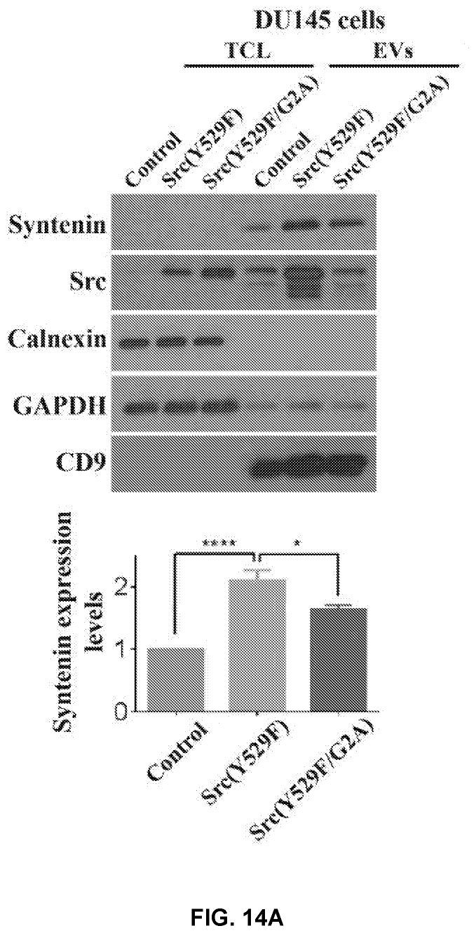

Lipid rafts are membrane-associated microdomains enriched with cholesterol and saturated phospholipids like sphingolipids. Lipid rafts are one of the essential pathways to mediate the encapsulation of proteins into EVs (Tan S S, et al. J Extracell Vesicles. 2013 2:22614; Trajkovic K, et al. Science. 2008 319:1244-7). To examine if lipid rafts mediate the encapsulation of Src kinase into EVs, cells were treated with Filipin III, a lipid raft disruption agent and cholesterol levels significantly decreased (FIG. 13). However, expression levels of Src kinase in EVs did not significantly change with Filipin III treatment in PC3 or DU145 cells (FIG. 7A), suggesting that the encapsulation of Src kinase into EVs is not regulated via the lipid raft mediated pathway.

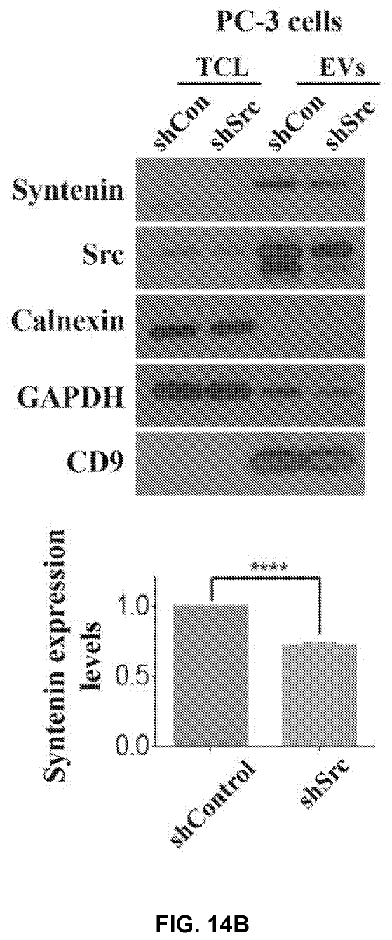

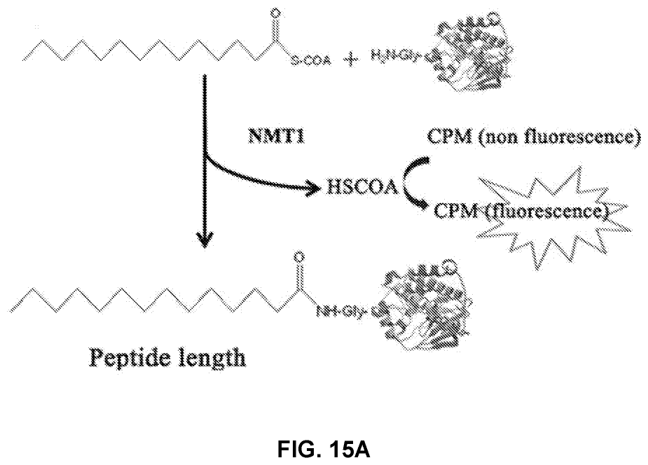

Syntenin is an important protein to mediate the EVs biogenesis, and is also enriched in EVs. Over-expression of Src(Y529F) in DU145 cells significantly increased levels of syntenin in EVs (FIG. 14A), but not in those cells expressing Src(Y529F/G2A) mutant. Additionally, knockdown of Src decreased expression levels of syntenin in EVs (FIG. 14B).

Syntenin is involved in multi-vesicular bodies (MVB) formation and the ESCRT-mediated biogenesis (Thery C, et al. Nat Rev Immunol. 2002 2:569-79). To further study if Src encapsulation into EVs is regulated by the ESCRT pathway, TSG101, an essential protein in the ESCRT pathway was knocked down in PC3 or 22Rv1 cells. Down-regulation of TSG101 did not change cellular levels of Src protein, but significantly decreased its levels in EVs (FIGS. 7B-7C). Collectively, the results suggest that the syntenin-ESCRT pathway is involved in encapsulation of active, myristoylated Src into EVs.

Discussion

The disclosed studies have demonstrated that myristoylation mediates the encapsulation of Src kinase into EVs. Myristoylation is one of the important lipid modifications for a panel of proteins (Resh M D. Biochimica et biophysica acta. 1999 1451:1-16). At least 182 proteins, which accounts for about 0.9% of the mammalian genome, possess an N-terminal glycine that is required for myristoylation. As shown herein, these potentially myristoylated proteins occur more frequently in EVs according to proteomic studies. Among the identified proteins, Src kinase is experimentally confirmed to be myristoylated (Kim S, et al. J Biol Chem. 2017). Src kinase is detected and/or enriched in EVs from all four tested prostate cancer cell lines, which is consistent with a report about expression levels of Src kinase in EVs (DeRita R M, et al. J Cell Biochem. 2017 118:66-73). Loss of myristoylation significantly inhibits Src or Fyn levels in EVs. Myristoylation allows for the association of Src kinase with the cell membrane (Kim S, et al. J Biol Chem. 2017), which is important for its biogenesis in EVs. In an analysis of proteins containing a myristoylation epitope that is fused to the N-terminus of GFP, loss of myristoylation in Acyl(G2A)TyA-GFP and Gag(G2A)TyA-GFP suppresses their encapsulation into the secreted vesicles or HIV virus (Shen B, et al. J Biol Chem. 2011 286:14383-95). Therefore, taking advantage of the fact that myristoylated proteins could preferentially be encapsulated into EVs, this fatty acyl modification might be considered as a strategy for delivery of proteins using EVs.

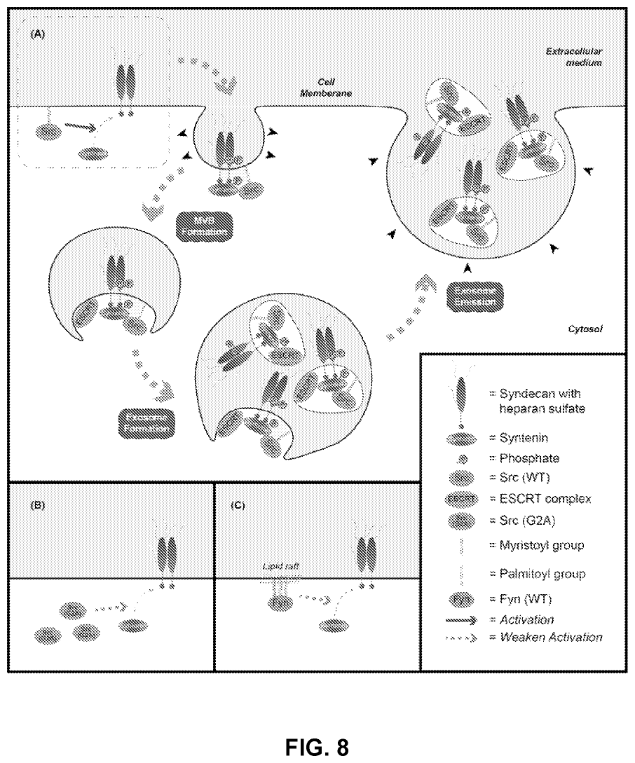

Myristoylation facilitating the encapsulation of Src kinase into EVs relies on two intertwined factors. First, myristoylation confers the association of Src kinase with the cell membrane to mediate the protein-protein interactions with other membrane-bound proteins (FIG. 8). In addition, myristoylation also regulates Src kinase activity, which could modulate phosphorylation of important proteins in EVs biogenesis. Due to the presence of membrane-bound phosphatases, the association of Src kinase with the cell membrane promotes the dephosphorylation of Src kinase at Tyr529, thereby activating Src kinase (Patwardhan P, et al. Mol Cell Biol. 2010 30:4094-107). The activated Src kinase exhibits better interaction with membrane proteins in comparison with wild type Src kinase (Shvartsman D E, et al. J Cell Biol. 2007 178:675-86). For example, syntenin is an important element to initiate ESCRT-mediated EVs biogenesis. Src kinase could interact with syndecan-syntenin for endosomal trafficking by regulating the phosphorylation of Y46 in syntenin (Imjeti N S, et al. Proc Natl Acad Sci. 2017 114:12495-500). Additionally, Src kinase also mediates phosphorylation of the DEGSY motif of syndecan-4 protein, which enhances syndecan binding to syntenin (Morgan M R, et al Dev Celt 2013 24:472-85). Loss of myristoylation inhibits the association of Src kinase with the cell membrane as well as its kinase activity (Kim S, et al. J Biol Chem. 2017). Consistently, the disclosed data indicate that constitutively active Src kinase is found at higher levels of syntenin in EVs compared to wild type Src. Suppression of Src levels or activity result in lower levels of syntenin in EVs, which might have inhibited syntenin mediated EVs biogenesis. Reciprocally, suppression of syntenin or the ESCRT pathway by down-regulation of TSG101, an essential player in the ESCRT-mediated protein trafficking, leads to inhibition of Src encapsulation to EVs. Therefore, myristoylation mediated Src encapsulation likely interacts with the syndecan-syntenin-ESCRT pathway in EVs biogenesis (FIG. 8).

As disclosed herein, encapsulation of Src kinase members into EVs is suppressed by palmitoylation at the N-terminus. Gain of palmitoylation sites in Src(S3C/S6C) mutant significantly reduced its levels in EVs. In contrast, removal of palmitoylated sites in Fyn(C3S/C6S) mutant significantly increased Fyn encapsulation into EVs. Loss or gain of palmitoylation in Src family kinase members can potentially change their kinase activity and oncogenic potential (Cai H, et al. Proc Natl Acad Sci USA. 2011 108:6579-84). Therefore, on one hand, palmitoylation suppressing the encapsulation of Src into EVs might be due to a reduction of Src kinase activity, thereby inhibiting the activation of syndecan-syntenin-ESCRT pathway as described in the above. On the other hand, the differential lipidation in myristoylation with/without palmitoylation could considerably change the localization of SFKs members in the cell membrane and the intracellular trafficking pathways (Sato I, et al. J Cell Sci. 2009 122:965-75; Sandilands E, et al. J Cell Sci. 2007 120:2555-64). For example, palmitoylation promotes SFK members localized at the lipid raft and caveolae region of the cell membrane (Shenoy-Scaria A M, et al. J Cell Biol. 1994 126:353-64). Deviation of palmitoylated SFKs members such as Fyn kinase toward the caveolae concentrated domain in the cell membrane could likely regulate their encapsulation into EVs.

Given the fact that expression levels or activity of Src kinase is usually dys-regulated in numerous cancers including prostate cancer (Irby R B, et al. Oncogene. 2000 19:5636) and metastatic castration resistant prostate cancer (Drake J M, et al. Proc Natl Acad Sci USA. 2013 110:E4762-9), the detection of myristoylated Src in the plasma EVs may potentially serve as an early biomarker for aggressive tumors. The number of EVs in urine or plasma are usually higher in cancer patients and correlated with a high Gleason score and metastatic prostate cancer patients (Vlaeminck-Guillem V. Front Oncol. 2018 8:222). Besides the number of EVs, the components of EVs including lipid, proteins, mRNA, microRNA, long non-coding RNAs and others have also been considered as potential biomarkers (Skog J, et al. Nat Cell Biol. 2008 10:1470-6). This study demonstrates that myristolated proteins, in particular myristoylated Src kinase, could potentially reflect Src-driven xenograft tumors by the detection of Src levels in the plasma EVs. This is supported by the evidence that Src is detected in the plasma EVs of TRAMP mice, a Src driven prostate tumor progression model (DeRita R M, et al. J Cell Biochem. 2017 118:66-73). Additionally, there is a report that an increase of c-Src levels is observed in EVs from multiple myeloma and immunoglobulin light chain (AL) amyloidosis (Di Noto G, et al. PLoS One. 2013 8:e70811). Future studies should explore whether Src or myristoylated Src levels in the plasma EVs from prostate cancer patients reflect tumor progression, which could potentially provide a biomarker of non-invasively monitoring aggressive prostate cancer.

### Example 2: Genetical Engineering Cas9 to Encapsulate CRISPR System into Extracellular Vesicles by Protein Myristoylation

Material and Methods

Plasmid constructs: To create non-lentiviral vector expressing myristoylated Cas9 (mCas9), Cas9-Guide or Cas9-Scramble CRISPR vectors (OriGene, Rockville, Md., USA) were used as the PCR template. The Src(WT; 8 a.a) (Forward primer) and mCas9 primer (reverse primer) (Table 6) were used to obtain a PCR product, which fused the DNA sequence of the first eight amino acid sequence in the N-terminus of Src kinase with the N-terminus of Cas9 gene. The obtained PCR product, and Cas9/sgRNA-Guide or Cas9/sgRNA-Scramble vectors, and were digested with BglII and BstZ171. After the ligation of PCR product and digested parental vector, non-viral vector, mCas9/sgRNA-Guide and mCas9/sgRNA-Scramble were created. To generate mCas9(G2A) vectors, a PCR product was generated using the created mCas9 vector as the DNA template, and Src(G2A;8a.a) (forward primer) and mCas9 primer (reverse primer). The obtained PCR product were cloned into at the BglII and BstZ171 sites. To generate Cas9/sgRNAs in the bicistronic vector to target GFP gene, three set of sgRNA primers were designed and commercially synthesized (Table 6). The annealed products were cloned into the above vectors between the BamHI and BsmBI sites. As a result, Cas9/sgRNA-GFP, mCas9/sgRNA-GFP, and mCas9(G2A)/sgRNA-GFP were created. All DNA constructs were verified by sequencing.

TABLE 6
Primer sequences used for cloning Src mutants, sgRNA-GFP on Cas9 vectors

| Gene | Direction | Sequence (5′-3′) |
|---|---|---|
| Src | Forward | CATAGATCTGCCGCCGCGATCGCCATGGGCAGCAACAAGAG |
| (WT; 8 a.a) | | CAAGCCCAAGGATAAGAAATACTCAATAGGACTGGATATTGG |
| | | (SEQ ID NO: 384) |
| Src | Forward | CATAGATCTGCCGCCGCGATCGCCATGGCCAGCAACAAGAG |
| (G2A; 8 a.a) | | CAAGCCCAAGG (SEQ ID NO: 385) |
| Src | Forward | CATAGATCTGCCGCCGCGATCGCCATGGGCTGCAACAAGAG |
| (S3C; 8 a.a) | | CAAGCCCAAGG (SEQ ID NO: 386) |
| Src | Forward | CATAGATCTGCCGCCGCGATCGCCATGGGCAGCAACAAGTG |
| (S6C; 8 a.a) | | CAAGCCCAAGG (SEQ ID NO: 387) |
| Src | Forward | CATAGATCTGCCGCCGCGATCGCCATGGGCTGCAACAAGTG |
| (S3C/56C) | | CAAGCCCAAGG (SEQ ID NO: 388) |
| mCas9 | Reverse | CATGTATACCTTCTCCTAGCTGTCCG (SEQ ID NO: 389) |
| sgRNA-GFP1 | Forward | GATCGGGGCGAGGAGCTGTTCACCGG (SEQ ID NO: 390) |
| | Reverse | AAAACCGGTGAACAGCTCCTCGCCCC (SEQ ID NO: 391) |
| sgRNA-GFP2 | Forward | GATCGGAGCTGGACGGCGACGTAAAG (SEQ ID NO: 392) |
| | Reverse | AAAACTTTACGTCGCCGTCCAGCTCC (SEQ ID NO: 393) |
| sgRNA-GFP3 | Forward | GATCGGGCCACAAGTTCAGCGTGTCG (SEQ ID NO: 394) |
| | Reverse | AAAACGACACGCTGAACTTGTGGCCC (SEQ ID NO: 395) |
| sgRNA- | Forward | GATCGACAACTTTACCGACCGCGCCG (SEQ ID NO: 396) |
| Luciferase | Reverse | AAAACGGCGCGGTCGGTAAAGTTGTC (SEQ ID NO: 397) |
| Luciferase-T7 | Forward | AAATTGCTTCTGGTGGCGC (SEQ ID NO: 398) |
| | Reverse | CGTCTTCGTCCCAGTAAGCT (SEQ ID NO: 399) |
| U6-Cas9 | Forward | GGACTATCATATGCTTACCGTAAC (SEQ ID NO: 400) |
| primers | Reverse | CATGTATACCTTCTCCTAGCTGTCCG (SEQ ID NO: 401) |

To generate lentivirus-based Cas9/sgRNA vectors, FlinkW lentiviral vector was used as a parental vector. First, FlinkW was digested by EcoRI and HpalI enzymes. The above non-lentiviral mCas9 or Cas9/sgRNA vectors were digested with EcoRI and PmeI sites, which generated two DNA fragments, one fragment with 1 kb (both ends are EcoR1) and the other fragment 4 kb (ECoR1 in 5′-end and Pme1 in 3′-end). The 4 kb fragment DNA was then inserted into the digested FlinkW lentiviral vector. After confirmed by sequencing, 1 kb fragment was further inserted into the above vector. Therefore, the 5 Kb of DNA fragment containing mCas9/sgRNA derived from non-viral vector was cloned into Flink W lentiviral vector.

Additionally, lentiviral vectors expressing Src(WT), Src(G2A), Src(Y529F), and Src(Y529F/G2A) were cloned into the FUCRW parental lentiviral vector. The lentivirus were generated from these lentiviral vectors to create stable cell lines.

Cell lines: SYF1 (Src^{-/-}Fyn^{-/-}Yes^{-/-}), 3T3, and human prostate cancer cell lines including DU145, PC3, 22Rv1, and LNCaP were purchased from American Type Culture Collection (ATCC). The cells were grown in the medium recommended by ATCC. Mycoplasma contamination was examined periodically. The cells were used up to 20 passages.

Isolation of EVs and characterization: To isolate EVs from the cell culture medium, the cell lines were grown in ATCC recommended medium in a 150-mm petri-dish. After reaching 90% confluence, the medium was replaced with fresh medium containing 5% exosome-free FBS (Life Technology Inc.), and grown in 5% CO₂ 37° C. incubator for another 24 h. The conditioned medium was collected for the EVs isolation. Specifically, the conditioned medium was repeatedly centrifuged at 4° C. at 300×g for 10 min, 2,000×g for 10 min, and 10,000×g for 30 min to remove live cells, dead cells, and cell debris, respectively. The supernatant was further ultra-centrifugated with 100,000×g at 4° C. for 90 min. The EVs pellet was re-suspended in 1×PBS to wash out the residual medium, and re-centrifugated at 100,000×g at 4° C. for 90 min. The pelleted EVs were re-suspended either in RIPA buffer for protein analysis or 1×PBS for Dynamic Light Scattering (DLS) analysis. The size, zeta potential, and concentration of EVs were measured by nanoparticle tracking analysis (NTA, Particle Metrix, Germany) with ZetaView software for data record and analysis.

Protein concentration determination: The protein concentration of EVs and cell lysates was determined by detergent compatible (DC) protein assay (Bio-Rad Laboratories). The total cell lysates (TCL) and EVs were dissolved in RIPA buffer [50 mM Tris-base (pH 7.4), 1% NP-40, 0.50% sodium deoxycholate, 0.1% SDS, 150 mM NaCl, 2 mM EDTA and protease inhibitor (1×)] and the manufacturer's protocol was followed.

Antibodies and Western blotting analysis: The total cell lysate and EVs dissolved in RIPA buffer were subjected to the standard immunoblotting analysis. The following antibodies were used: rabbit anti-Src (Cat #: 2109), rabbit anti-calnexin (Cat #: 2679), rabbit anti-CD-9 (Cat #: 13403 for human species, Cat #: 2118 for mouse species), rabbit anti-GAPDH (Cat #: 13403), rabbit anti-Fyn (Cat #: 4023), and rabbit anti-FAK (Cat #: 13009), rabbit CD81 (Cat #: 10037) were purchased from Cell Signaling Technology; rabbit anti-RFP (Cat #: 600-401-379, Rockland Inc), rabbit anti-AR (Cat #: sc-816, Santa Cruz Biotechnology), and secondary Antibody anti-rabbit IgG HRP (Cat #: 7074, Cell Signaling Technology) were used according to manufactory's recommended dilution. The band intensity was quantified by Image J software.

Computational docking analysis: The docking analysis of NMT1 with the first amino acid, and a leading peptide containing the first 2, 3, 4, 5, 6, 7, 8, 9, or 10 amino acids from c-Src, indicates that a peptide with 7-8 amino acids has favorable docking with NMT1 enzyme (lower score).

NMT1 activity assay: NMT1 catalyzes the incorporation of the myristoyl group into the N-terminus of the glycine in an octapeptide, such as Gly-Ser-Asn-Lys-Ser-Lys-Pro-Lys derived from the leading sequence of Src kinase, designated as Src8(WT), and releases CoA. The amount of the released CoA were reacted with 7-diethylamino-3-(4′-maleimidylphenyl)-4-methylcoumarin. The assay was performed in 96-well black microplates. The produced fluorescence intensity was measured by Flex Station 3, and detected by microplate reader (excitation at 390 nm; emission at 479 nm). To measure the Km and Vmax of NMT1 which catalyzed various octapeptides substrates derived from various proteins, twenty-five octapeptides were synthesized by GenScript. These peptide included Src8(G2A), a mutant octapeptide [Ala-Ser-Asn-Lys-Ser-Lys-Pro-Lys, SEQ ID NO: 383], which is not a substrate of NMT1 enzyme. Each data point has three repeats.

Determination of myristoylated Src kinase by Click chemistry: Cells expressing Src kinase were grown until 90% confluence in EMEM medium with 5% FBS. The medium was replaced with EMEM medium containing exosome-free FBS and 50 μM of myristic acid-azide (an analog of myristic acid) and the cells were grown for another 24 h. The conditioned medium was collected and used for EVs isolation as described above. The cells or EVs were lysed in M-PER buffer (Thermo Scientific) containing protease inhibitors and phosphatase inhibitors. The cell lysates or EVs lysate (10 μg protein) were added to a working solution containing biotin-alkyne (0.1 mM), CuSO₄ (1 mM), TCEP (1 mM) and TBTA (0.1 mM) and incubated at room temperature for 1 h. After the Click reaction, the samples were mixed with loading dye and boiled at 95° C. for 5 min. The lysates were subjected to SDS-PAGE and transferred to a nitrocellulose membrane. After blocking with 5% milk overnight, the membrane was incubated with High Sensitivity Streptavidin-HRP (catalog No. 21130, ThermoFisher Scientific) at room temperature for 1 h. Myristoylated proteins (e.g., myristoylated Src kinase) were detected by ECL.

Alternatively, myristoylated Src or Cas9 were detected by antibody against myristoylated octapeptide derived from Src kinase. To Develop an antibody to detect myristoylated protein, particularly the proteins containing an octapeptide Gly-Ser-Asn-Lys-Ser-Lys-Pro-Lys (SEQ ID NO: 367) in the N-terminus, such as Src kinase or the octapeptide fused Cas9, Myristoyl-Gly-Ser-Asn-Lys-Ser-Lys-Pro-Lys (SEQ ID NO: 367) was synthesized as an antigen by GenScript, and injected into two rabbits (4857 and 4858) to generate antibodies. After 3^{rd} immunization, the antibody was purified using myristoylated octapeptide antigen. The reactivity was measured by ELISA assay using myristoylated octapeptide and non-myristoylated octapeptide.

Statistical analysis: The data are presented as mean±SEM (standard error of the mean). All the data with more than two groups were analyzed by one-way ANOVA with a post hoc Tukey test in GraphPad Prism software, and two values were compared by an unpaired student t-test. * p<0.05; ** p<0.01; *** p<0.001; NS: not significant.

Results

The Octapeptide Derived from Src Kinase was a Favorable Substrate of N-Myristoyltransferase 1.

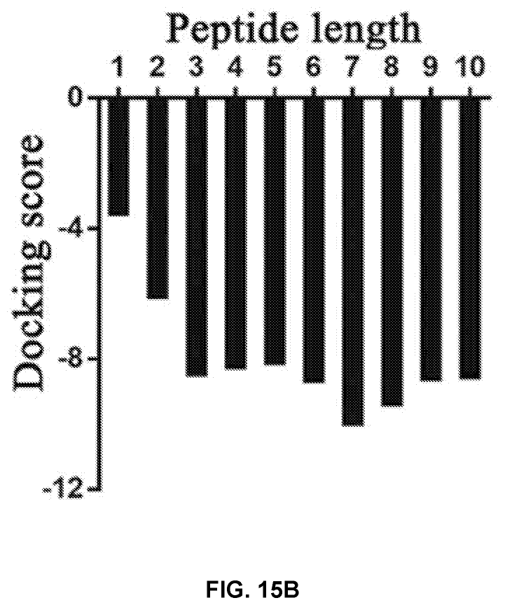

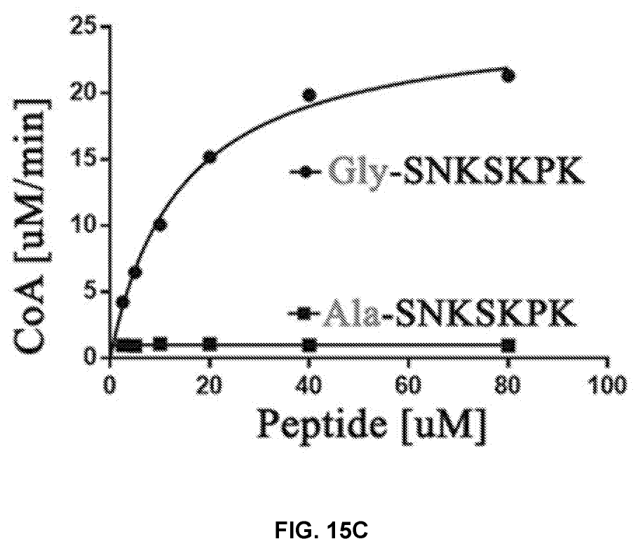

Protein myristoylation is catalyzed by N-myristoyltransferase (NMT) (41). Two mammalian isozymes of NMTs, NMT1 and NMT2 (77% identity), catalyze this myristoylation process. NMT1/2 binds myristoyl-CoA and transfers the myristoyl group to an N-terminal glycine with release of CoA (43) (FIG. 15A). We have previously purified and crystalized the truncated NMT1 protein (without the N-terminus inhibitory domain) and have identified the myristoyl-CoA binding and peptide binding sites of NMT1. To better characterize the NMT1 function, the full length NMT1 protein was constructed and both myristoyl-CoA and peptide binding sites were identified; the minimal energy required for docking with an amino acid to different length of peptides (from 2-10 amino acids peptide) was determined. Based on computational docking analysis, a 7-8 amino acid peptide has the lower docking score (FIG. 15B). Octapeptide showed numerous favorable interaction with NMT1. Twenty-five representative octapeptides (based from the docking score) derived from the N-terminus of myristoylated proteins were further examined to determine the feasibility as an NMT1 substrate (Table 7). The octapeptide derived from Src kinase, designated to Src8(WT), but not Src8(G2A), was among the best substrate of NMT1 (FIG. 150 and Table 7). Together, the octapeptide derived from Src kinase containing Gly in the N-terminus is one of candidates to serve as an epitope tag of protein myristoylation.

The feasibility of twenty-six octapeptides served as a substrate of N-myristoyltransferase 1 (Table 7). Octapeptides derived from the leading sequence of 25 myristoylated proteins with glycine at the N-terminus together with a mutation of octapeptide from Src kinase, called Src(G2A), were examined for their feasibility as an NMT1 substrate using the NMT1 activity assay (described in Material and Methods). Km and Vmax catalyzed by full length NMT1 protein were calculated. The docking score was analyzed based on the re-constructed full length NMT1 protein structure. Count means that a particular protein was detected in EVs from cancer cells among 60 cell lines by Mass spectrometry.

TABLE 7
Octapeptide substrates of N-myristoyltransferase 1

| Protein | Peptide | | Docking | Km | Vmax |
|---|---|---|---|---|---|
| Name | sequence (8 Residues) | Count | Score | [uM] | (uM/min) |
| YES1 | GCIKSKEN (SEQ ID NO: 358) | 54 | -12.6 | 14.4 | 61.0 |
| FYN | GCVQCKDK (SEQ ID NO: 359) | 10 | -12.3 | 5.2 | 54.9 |
| MARCKS | GAQFSKTA (SEQ ID NO: 360) | 46 | -11.7 | 38.4 | 6.4 |
| MARCKSL1 | GSQSSKAP (SEQ ID NO: 361) | 47 | -11.2 | 11.7 | 6.6 |
| NOL3 | GNAQERPS (SEQ ID NO: 362) | 24 | -11.2 | 1.4 | 2.0 |
| NAA40 | GRKSSKAK (SEQ ID NO: 363) | 6 | -11.0 | 1.2 | 1.8 |
| PSMC1 | GQSQSGGH (SEQ ID NO: 364) | 60 | -11.0 | 40 | 9.6 |
| ZNRF2 | GAKQSGPA (SEQ ID NO: 365) | 4 | -10.9 | 2.0 | 1.6 |
| RNF11 | GNCLKSPT (SEQ ID NO: 366) | 4 | -10.6 | 16.7 | 61.1 |
| SRC | GSNKSKPK (SEQ ID NO: 367) | 42 | -10.5 | 14.3 | 25.8 |
| LYN | GCIKSKGK (SEQ ID NO: 368) | 47 | -9.6 | 22.5 | 64.7 |
| SCYL3 | GSENSALK (SEQ ID NO: 369) | 1 | -9.2 | 0.8 | 1.7 |
| FRS2 | GSCCSCPD (SEQ ID NO: 370) | 3 | -8.2 | 28.2 | 54.7 |
| RP2 | GCFFSKRR (SEQ ID NO: 371) | 47 | -6.0 | 13.6 | 60.8 |
| LNP | GGLFSRWR (SEQ ID NO: 372) | 5 | -6.0 | 10.3 | 21.9 |
| NDUFAF4 | GALVIRGI (SEQ ID NO: 373) | 3 | -5.8 | 0.5 | 1.2 |
| REP15 | GQKASQQL (SEQ ID NO: 374) | 1 | -5.4 | 15.7 | 3.4 |
| GNAZ | GCRQSSEE (SEQ ID NO: 375) | 2 | -5.3 | 15.7 | 64.4 |
| LANCL2 | GETMSKRL (SEQ ID NO: 376) | 15 | -5.1 | 13.0 | 5.3 |
| DEGS1 | GSRVSRED (SEQ ID NO: 377) | 3 | -5.0 | 79.2 | 12.9 |
| ARL6 | GLLDRLSV (SEQ ID NO: 378) | 2 | -4.9 | <0.1 | 1.8 |
| ARF6 | GKVLSKIF (SEQ ID NO: 379) | 60 | -3.5 | 4.4 | 13.6 |
| ARL2 | GLLTILKK (SEQ ID NO: 380) | 50 | -3.4 | 0.4 | 1.2 |
| NDUFB7 | GAHLVRRY (SEQ ID NO: 381) | 3 | No Score | 16.4 | 2.8 |
| DDX46 | GRESRHYR (SEQ ID NO: 382) | 24 | No Score | <0.1 | 2.0 |
| SRC(G2A) | ASNKSKPK (SEQ ID NO: 383) | N/A | N/A | <0.1 | 1.0 |

Fusion of Octapeptide to the N-Terminus of Cas9 Maintained its Genome Editing Function, and Promoted Cas9 Protein to be Encapsulated into EVs.

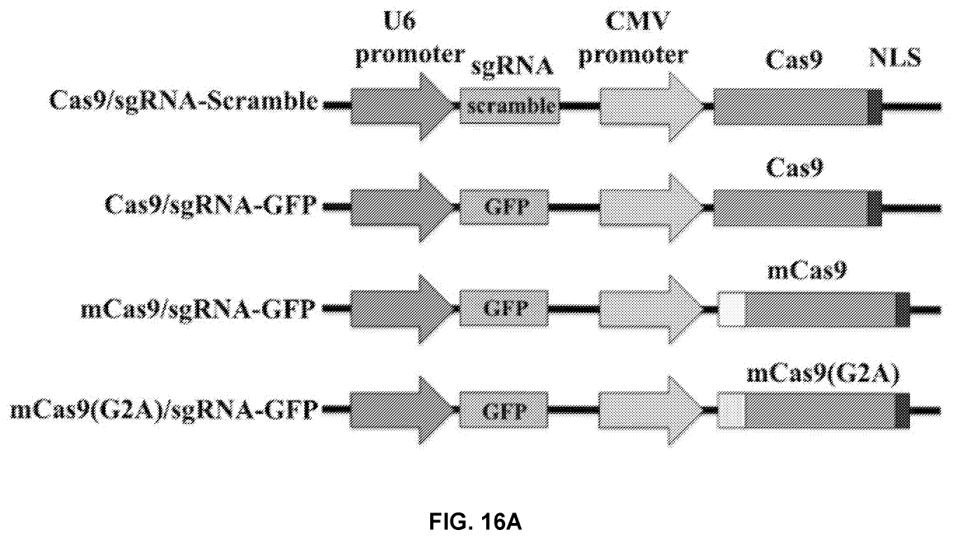

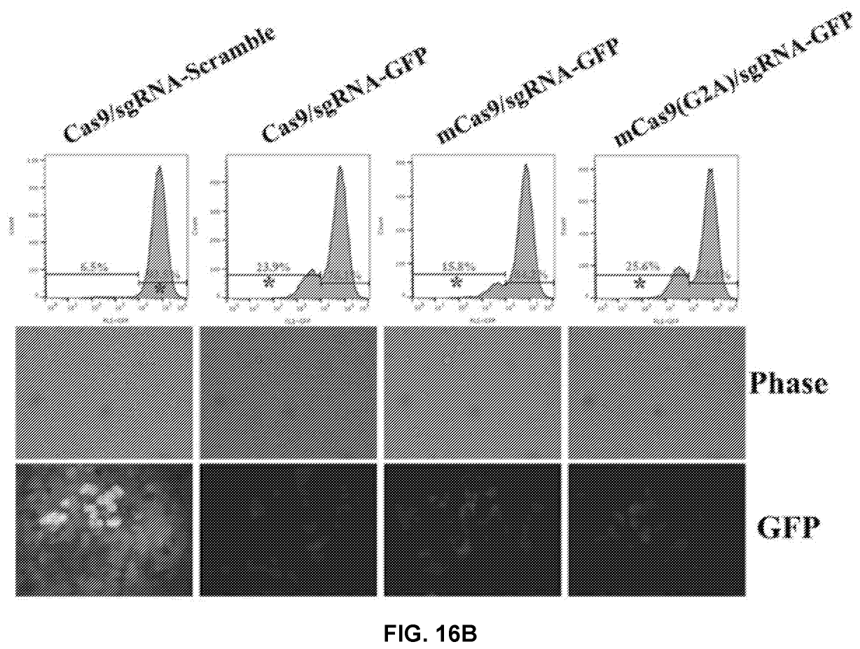

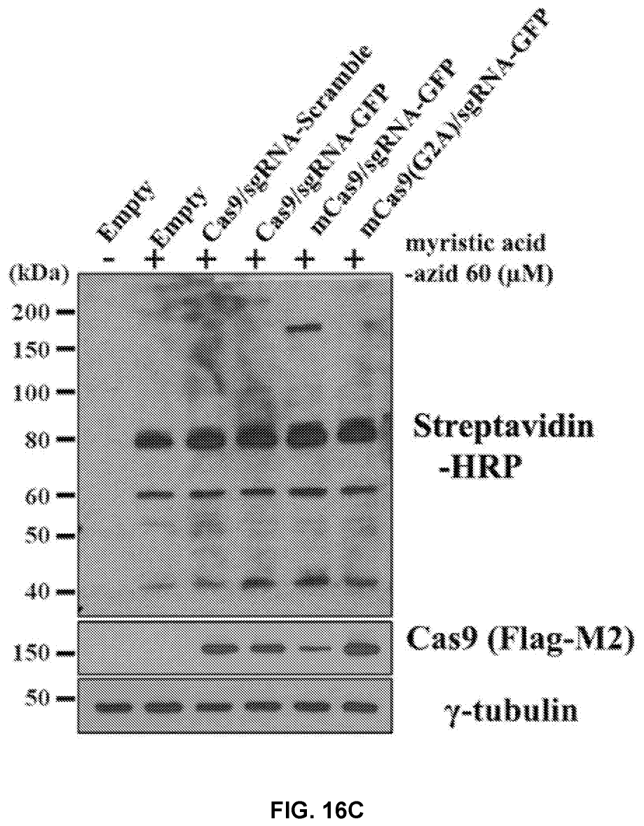

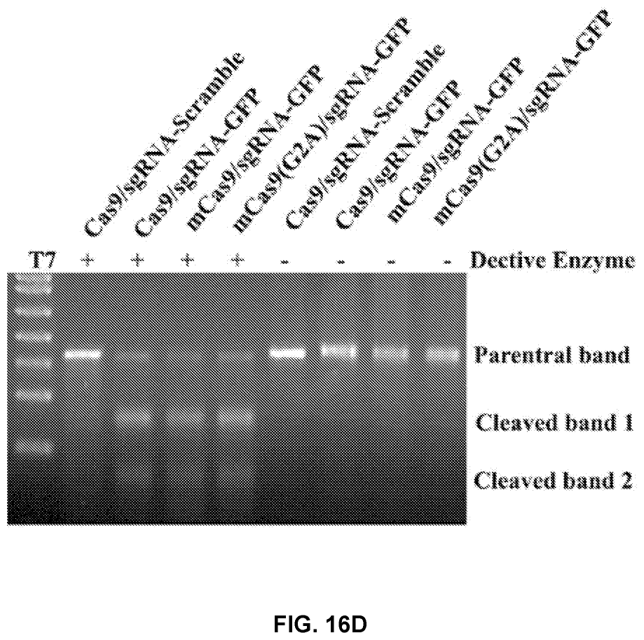

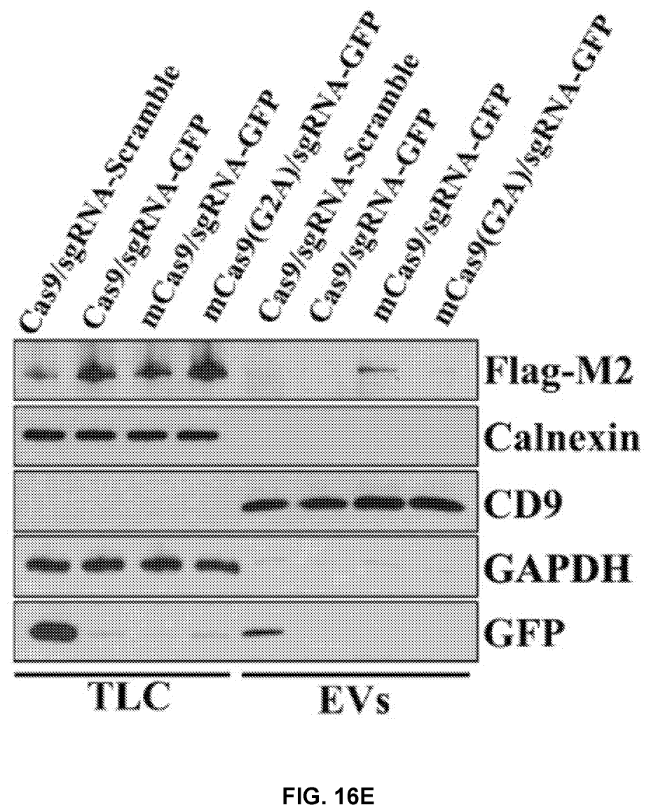

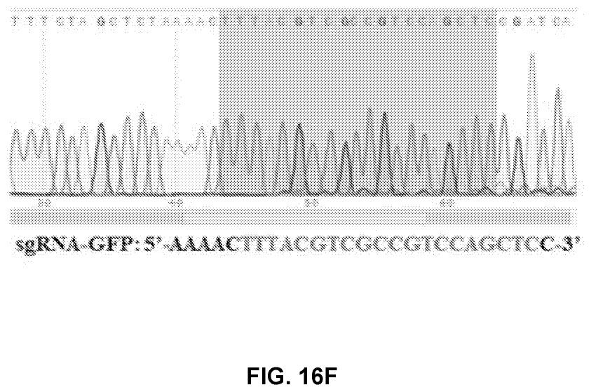

To this end, a favorable octapeptide derived from the leading sequence of Src kinase was identified as a NMT1 substrate. To fuse the octapeptide to the N-terminus of Cas9, a bi-cistronic lentiviral vector expressing Cas9 and sgRNA (no target), or myristoylated Cas9 or non-myristoylated Cas9, designated as mCas9 or mCas9(G2A) and sgRNA targeting GFP gene was generated, respectively (FIG. 16A). 293T-GFP cells were transduced with Cas9/sgRNA-scramble, Cas9/sgRNA-GFP, mCas9/sgRNA-GFP, or mCas9(G2A)/sgRNA-GFP by lentiviral infection. In 293T-GFP cells treated with Cas9/sgRNA-Scramble group, it contained 6.5% of non-GFP cells (likely dead cells). 23.5%, 15.8%, and 25.6% of non-GFP cells were detected in 293T-GFP cells expressing Cas9/sgRNA-GFP, mCas9/sgRNA-GFP, mCas9(G2A)/sgRNA-GFP, respectively (FIG. 16B). The non-GFP stable cell lines were isolated by FACS sorting. While Cas9 expression was detected in cell lines expressing Cas9/sgRNA-Scramble, Cas9/sgRNA-GFP, mCas9/sgRNA-GFP, or mCas9(G2A)/sgRNA-GFP, only myristoylated Cas9 was detected in cells expressing mCas9/sgRNA-GFP (FIG. 16C). Genome editing of GFP gene was further confirmed by T7 analysis in the non-GFP stable cell lines (EVs-producing cells) (FIG. 16D). EVs-producing cells were further expanded, and EVs were collected from these cells. Only EVs derived from EVs-producing cells expressing mCas9, but not un-modified Cas9 or mCas9(G2A) expressing Cas9 (FIG. 16E). Total RNA from EVs were also extracted, and sgRNA was detected in EVs derived from EV-producing cells expressing mCas9, but not un-modified Cas9 or mCas9(G2A). The sequence of sgRNA targeting GFP together with scaffold sgRNA was verified by the Sanger sequencing analysis (FIG. 16F). Taken together, myristoylated Cas9 and sgRNA-GFP were encapsulated into EVs, and protein myristoylation resulting from the fusion of octapeptide with Cas9 is important for the encapsulation process.

Isolation of EVs-Producing Cells Expressing mCas9/sgRNA-Luciferase, and Encapsulation of mCas9/sgRNA-Luciferase into EVs.

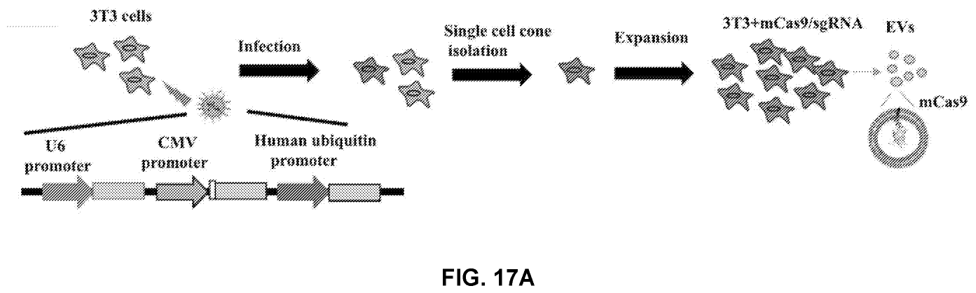

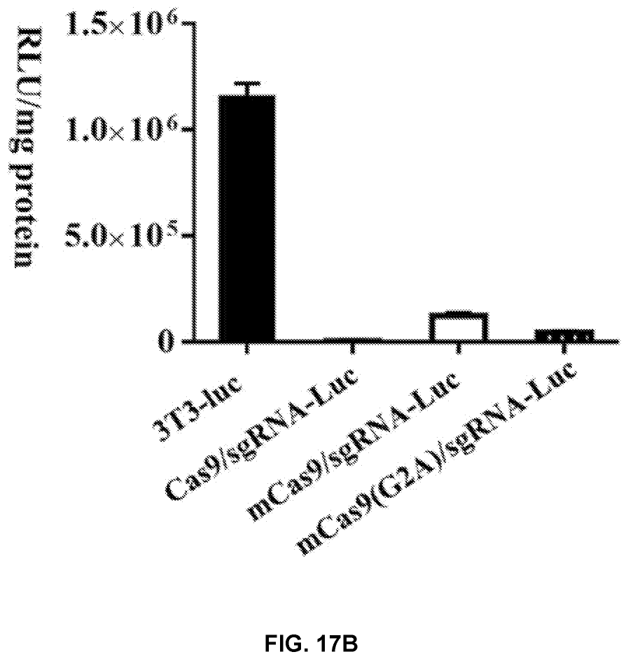

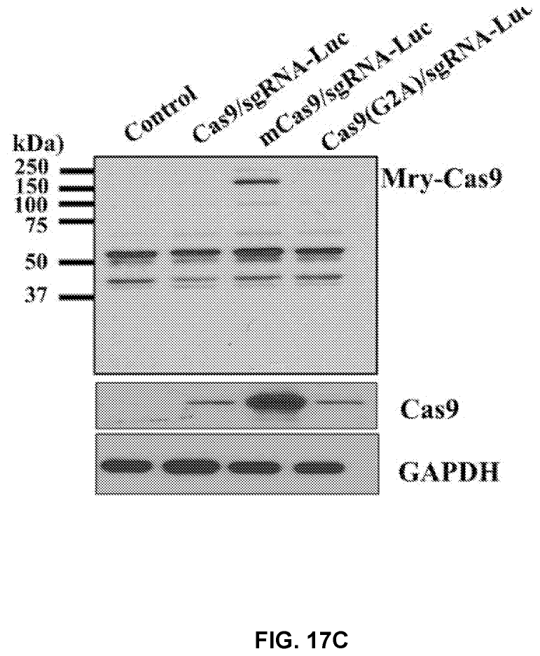

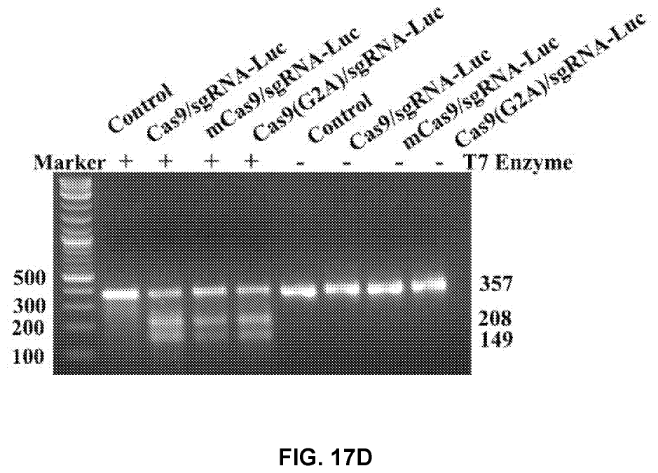

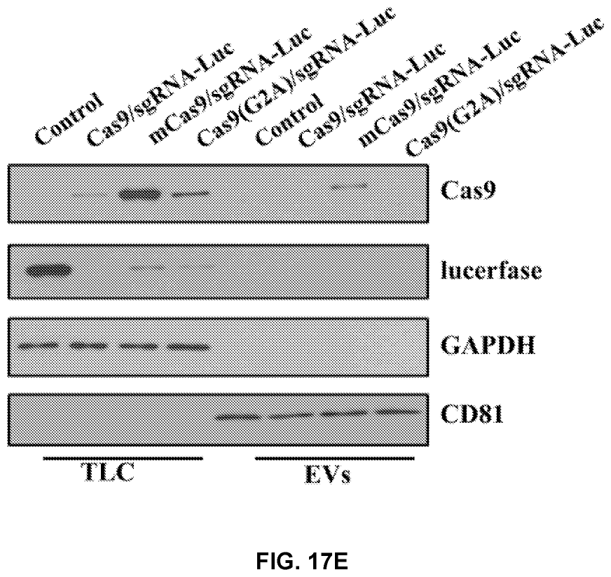

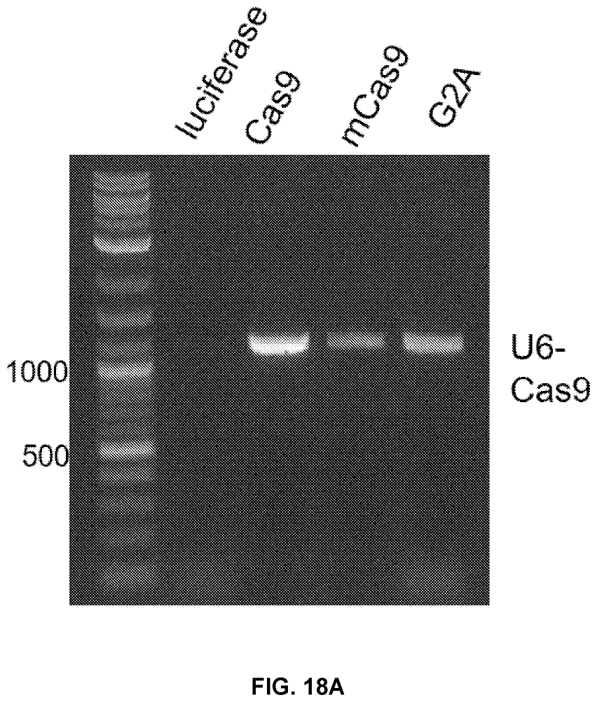

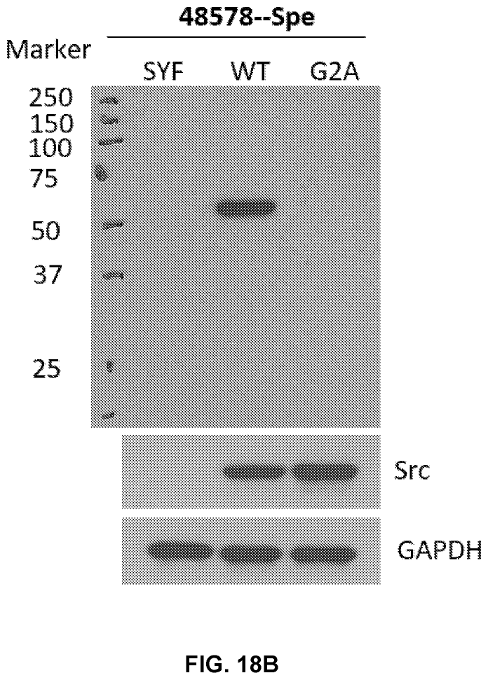

Using the similar approach, lentiviral vector expressing Cas9/sgRNA-luciferase (luc), mCas9/sgRNA-Luc, or mCas9(G2A)/sgRNA-Luc was generated. To create EVs-producing 3T3 cells, 3T3 cells expressing luciferase gene were transduced with Cas9, mCas9, or mCas9(G2A)/sgRNA-Luc by lentiviral infection. Single cell clones transduced with Cas9, mCas9, or mCas9(G2A)/sgRNA-Luc was isolated through dilution in the 96-well plate (FIG. 17A). The isolated cell clone showed Cas9 expression and down-regulation of luciferase activity in EVs-producing cells expressing Cas9, mCas9, or mCas9(G2A)/sgRNA-luciferase (FIG. 17B). The integration of Cas9, mCas9, or mCas9(G2A)/sgRNA-luciferase into the genomic DNA of the isolated EVs-producing cells were verified (FIG. 18A). Genome editing in targeting luciferase gene was confirmed by T7 endonuclease activity (FIG. 17C). A cell clone expressing mCas9/sgRNA-Luc was isolated, which expressed higher levels of Cas9 in comparison with those isolates expressing Cas9 and mCas9(G2A) (FIG. 17D). An antibody targeting myristoylated octapeptide) was developed, which was specifically detected myristoylated octapeptide (or myristoylated Src kinase or myristoylated Cas9) (FIG. 18B). Only myristoylated Cas9 was detected in EVs-producing cell expressing mCas9, but not Cas9 or mCas9(G2A) (FIG. 17D). More importantly, Cas9 was only detected in EVs derived from EVs-producing cells expressing mCas9, but not Cas9 or mCas9(G2A) (FIG. 17E). The result suggests that myristoylation promotes mCas9 to encapsulate into EVs.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion protein, comprising a myristoylation domain, a Cas9 domain, and a nuclear localization signal, wherein the myristoylation domain does not comprises a palmitoylation motif, wherein the polypeptide is configured to be myristoylated during translation, to be encapsulated into exosomes, and to localize to the nucleus of recipient cells.

2. The fusion protein of claim 1, wherein the myristoylation domain comprises the amino acid sequence G-X1-X1-X1-S/T-X2-X2-X2 (SEQ ID NO:1), wherein X1 is any amino acid other than Cys, and wherein X2 is any amino acid or nothing.

3. A recombinant polynucleotide, comprising a nucleic acid sequence encoding a guide RNA operably linked to a first expression control sequence, and a nucleic acid sequence encoding the fusion protein of claim 1 operably linked to a second expression control sequence.

4. A cell comprising the polynucleotide of claim 3.

5. A method of making a gene editing composition, comprising culturing the cell of claim 4 under conditions suitable to produce extracellular vesicles encapsulating the guide RNA and fusion protein.

6. A gene editing composition, comprising extracellular vesicle encapsulating the fusion protein of claim 1 and a guide RNA.

7. The gene editing composition of claim 6 produced by the method of claim 6.

8. A method for editing a gene in a cell, comprising contact the cell with the gene editing composition of claim 6.

9. A method for encapsulating a protein into an extracellular vesicle, comprising providing a fusion of the protein with a myristoylation domain, wherein the myristoylation domain does not comprises a palmitoylation motif, wherein the polypeptide is configured to be myristoylated during translation and encapsulated into extracellular vesicles.
